(19) United States
(12) Patent Application Publication
(10) Pub. No.: US 2009/0028882 A1
(43) Pub. Date: Jan. 29, 2009

(54) Methods of inhibiting binding of beta-sheet fibril to rage and consequences thereof

(75) Inventors: David M. Stern, Augusta, CA (US); Shi Du Yan, New York, NY (US); Ann Marie Schmidt, Franklin Lakes, NJ (US)

Correspondence Address: John P. White;Cooper & Dunham LLP, 1185 Avenue of the Americas, New York, NY, 10036, US

(73) Assignee: The Trustees of Columbia University in the City of New York

(21) Appl. No.: 12/009,572

(22) Filed: Jan. 18, 2008

Related U.S. Application Data
(63) Continuation of application No. 10/049,893, filed on Jul. 22, 2002, now abandoned.
Application No. 10/049,893 is a continuation-in-part of application No. 09/374,213, filed on Aug. 13, 1999, now abandoned.

(51) Int. Cl. A61K 38/16; A61K 39/395; A61P 25/00

(52) U.S. Cl. 424/178.1; 514/12

(57) ABSTRACT

This invention provides a method of inhibiting the binding of beta-sheet fibril to RAGE on the surface of a cell which comprises contacting the cell with a binding-inhibiting amount of a compound capable of inhibiting binding of beta-sheet fibril to RAGE so as to thereby inhibit binding of beta-sheet fibril to RAGE.

In one embodiment, the beta-sheet fibril is amyloid fibril. In one embodiment, the compound is sRAGE or a fragment thereof. In another embodiment, the compound is an anti-RAGE antibody or portion thereof.

This invention provides the above method wherein the inhibition of binding of the beta-sheet fibril to RAGE has the consequences of decreasing the load of beta-sheet fibril in the tissue, inhibiting fibril-induced programmed cell death, and inhibiting fibril-induced cell stress.

This invention also provides methods of determining whether a compound inhibits binding of a beta-sheet fibril to RAGE on the surface of a cell.

This application is a continuation-in-part and claims priority of U.S. Ser. No. 09/374,213, filed Aug. 13, 1999, the contents of which are incorporated by reference.

The invention disclosed herein was made with Government support under grant numbers AG00690, AG14103, AG12891, NS31220, HL56881, HL69091 from the USPHS, JDFI and the Surgical Research Fund. Accordingly, the government has certain rights in this invention.

Throughout this application, various publications are referenced to within parentheses. Disclosures of these publications in their entireties are hereby incorporated by reference into this application to more fully describe the state of the art to which this invention pertains. Full bibliographic citations for these references may be found at the end of this application, preceding the claims.

### BACKGROUND OF THE INVENTION

Amyloid beta-peptide (Aβ) engagement of cell surface receptors would be expected to have diverse consequences for cell function. Constitutive production of low levels of Aβ, principally Aβ(1-40), throughout life suggests an homeostatic role for the peptide. This is consistent with neurologic abnormalities observed in mice deletionally mutant for β-amyloid precursor protein (βAPP)(Zheng et al., 1995). However, deposition of Aβ fibrils sets the stage for Alzheimer's disease (AD) in which accumulation of amyloidogenic material may be associated with neuronal toxicity and diminished synaptic density, ultimately leading to clinical dementia (Terry et al., 1991; Kosik, 1994; Funato et al., 1998; Selkoe, 1999). Mechanisms for removing and, potentially, detoxifying Aβ fibrils include possible uptake by the macrophage scavenger receptor on microglia (Khoury et al., 1996; Paresce et al., 1996), and endocytosis in complex with apoE and/or a₂-macroglobulin by receptors involved in cellular processing of lipoproteins (Aleshkov et al., 1997; LaDu et al., 1997; Narita et al., 1997). Another property of cell surface binding sites for Aβ could involve tethering fibrils to the cell surface, thereby enhancing cytotoxicity either directly (for example, Aβ by itself has been shown to generate reactive oxygen species) (Hensley et al., 1994), or indirectly, via triggering of signal transduction mechanisms (Yan et al., 1996; Gillardon et al., 1996; Yaar et al., 1997; Yan et al., 1997; Akama et al., 1998; Guo et al., 1998; Nakai et al., 1998; Combs et al., 1999). In the presence of large numbers of fibrils, late in AD, receptor-independent destabilization of membranes might be expected to predominate and could explain neuronal toxicity (Pike et al., 1993, Pollard et al., 1995 Mark et al., 1996). However, earlier in the disease, when fibrils are less frequently encountered and the Aβ burden is low, cellular receptors might engage nascent amyloid fibrils and magnify their biologic effects. In view of the capacity of Receptor for Advanced Glycation Endproduct or RAGE to bind soluble Aβ(Yan et al., 1996; Yan et al., 1997), it was considered whether such a receptor might interact with β-sheet fibrils composed of Aβ or other amyloid-forming monomers, activating signal transduction mechanisms and, thereby, augmenting cellular dysfunction in fibrillar pathologies.

RAGE is a multiligand member of the immunoglobulin superfamily of cell surface molecules. The receptor was first identified by its ability to bind nonenzymatically glycoxidized adducts of macromolecules termed Advanced Glycation Endproducts (AGEs) (Schmidt et al., 1999). As it was unlikely that RAGE was intended solely to interact with AGEs, we sought other ligands for the receptor. Amphoterin, a nonhistone chromosomal protein also associated with extracellular matrix, engages RAGE and induces receptor-dependent changes in cell migration (Hori et al., 1995). Furthermore, RAGE is the first-recognized receptor for S100/calgranulins (Hofmann et al., 1999), linking it to the pathogenesis of inflammation (increased expression of S100 proteins in AD brain has also been identified) (Marshak et al., 1992; Sheng et al., 1996). During studies to characterize the interaction of RAGE with these other ligands, it was found, quite unexpectedly, that RAGE bound Aβ(1-40/1-42) and served as a cofactor propagating Aβ-induced perturbation of cellular functions (Yan et al., 1996; Yan et al., 1997). However, since RAGE is expressed at low levels in normal mature brain, it was reasoned that its interaction with Aβ(1-40) under physiologic conditions was unlikely. With concurrent AD, one of the pathologic changes observed in neurons, microglia, astrocytes and affected cerebral vasculature is enhanced expression of RAGE (Yan et al., 1996; Yan et al., 1997). Thus, in an Aβ-rich environment, receptor-dependent facilitation of the assembly of Aβ oligomers and/or fibrils in proximity to the cell surface, followed by binding and triggering of signal transduction mechanisms, had the potential to provide a pathologic amplification mechanism in early stages of AD.

It is reported here that RAGE serves as a magnet to tether Aβ fibrils to the cell surface predominately via its V-domain, and that this causes receptor-mediated activation of the MAP kinase pathway, with resultant nuclear translocation of NF-kB, and, utilizing distinct intracellular mechanisms, receptor-dependent induction of DNA fragmentation. Furthermore, incubation of initially soluble Aβ with RAGE accelerates fibril formation. Consistent with the concept that RAGE interacts with β-sheet fibrils, RAGE binds fibrils composed of amyloid A, amylin, and prion-derived peptides, though the receptor does not interact with the soluble subunits. Engagement of RAGE by any of these fibrils results in receptor-dependent cellular activation. In a model of systemic amyloidosis, administration of an excess of soluble (s) RAGE, a truncated form of the receptor spanning the extracellular, ligand binding portion of the molecule, blocked cellular perturbation in the spleen. At these high concentrations, sRAGE had cytoprotective properties, acting as a decoy to prevent interaction of fibrils with cell surface RAGE, and suppressed splenic amyloid accumulation. These data suggest a new paradigm in which fibrils adopting a β-sheet structure are imbued with a key biologic property analogous to a “gain of function;” via binding to RAGE, they acquire the ability to magnify their effects by activating signal transduction mechanisms resulting in cellular perturbation.

The invention disclosed herein differs from that of prior work which did not discuss or disclose fibril. The conditions used in the prior work were such that fibril formation was not possible. The invention disclosed herein also differs from the prior work which taught that the binding was sequence specific. However, the data presented suggests that the binding is structure specific.

### SUMMARY OF THE INVENTION

This invention provides a method of inhibiting the binding of a β-sheet fibril to RAGE on the surface of a cell which comprises contacting the cell with a binding inhibiting amount of a compound capable of inhibiting binding of the β-sheet fibril to RAGE so as to thereby inhibit binding of the β-sheet fibril to RAGE. In one embodiment the β-sheet fibril is amyloid fibril.

In one embodiment, the compound is sRAGE or a fragment thereof. In another embodiment, the compound is an anti-RAGE antibody or portion thereof.

This invention provides the above method wherein the inhibition of binding of the β-sheet fibril to RAGE has the consequence of decreasing the load of β-sheet fibril in the tissue.

This invention provides the above method wherein the inhibition of binding of the β-sheet fibril to RAGE has the consequence of decreasing the load of β-sheet fibril in the tissue. This invention also provides the above method wherein the inhibition of binding of the β-sheet fibril to RAGE has the consequence of inhibiting fibril-induced programmed cell death. This invention further provides the above method wherein the inhibition of binding of the β-sheet fibril to RAGE has the consequence of inhibiting fibril-induced cell stress.

This invention provides a method of preventing and/or treating a disease involving β-sheet fibril formation other than Alzheimer's Disease in a subject which comprises administering to the subject a binding inhibiting amount of a compound capable of inhibiting binding of the β-sheet fibril to RAGE so as to thereby prevent and/or treat a disease involving β-sheet fibril formation other than Alzheimer's Disease in the subject.

This invention provides a method of determining whether a compound inhibits binding of a β-sheet fibril to RAGE on the surface of a cell which comprises:
(a) immobilizing the β-sheet fibril on a solid matrix;
(b) contacting the immobilized β-sheet fibril with the compound being tested and a predetermined amount of RAGE under conditions permitting binding of β-sheet fibril to RAGE in the absence of the compound;
(c) removing any unbound compound and any unbound RAGE;
(d) measuring the amount of RAGE which is bound to immobilized β-sheet fibril;
(e) comparing the amount measured in step (d) with the amount measured in the absence of the compound, a decrease in the amount of RAGE bound to β-sheet fibril in the presence of the compound indicating that the compound inhibits binding of β-sheet fibril to RAGE.

This invention provides a method of determining whether a compound inhibits binding of β-sheet fibril to RAGE on the surface of a cell which comprises:
(a) contacting RAGE-transfected cells with the compound being tested under conditions permitting binding of the compound to RAGE;
(b) removing any unbound compound;
(c) contacting the cells with β-sheet fibril under conditions permitting binding of β-sheet fibril to RAGE in the absence of the compound;
(d) removing any unbound β-sheet fibril;
(e) measuring the amount of β-sheet fibril bound to the cells;
(f) separately repeating steps (c) through (e) in the absence of any compound being tested;
(g) comparing the amount of β-sheet fibril bound to the cells from step (e) with the amount from step
(f), wherein reduced binding of β-sheet fibril in the presence of the compound indicates that the compound inhibits binding of β-sheet fibril to RAGE.

This invention provides a compound not previously known to inhibit binding of β-sheet fibril to RAGE determined to do so by the above methods.

This invention provides a method of preparing a composition which comprises determining whether a compound inhibits binding of β-sheet fibril to RAGE by the above methods and admixing the compound with a carrier.

### BRIEF DESCRIPTION OF THE FIGURES

FIG. 1. Interaction of RAGE with β-sheet fibrils. A-B. Binding of RAGE to immobilized soluble Aβ(1-40) (A) or preformed Aβ(1-40) fibrils (B). Freshly prepared synthetic Aβ(1-40) or preformed Aβ fibrils (5 μg/well of Aβ monomer equivalent in each case) was adsorbed to microtiter plates for 20 hrs at 4° C., excess sites in wells were blocked with albumin (1%), followed by addition of sRAGE for 2 hrs at 37° C. Unbound material was removed by washing, and bound sRAGE was determined by ELISA. Data was analyzed by nonlinear least squares analysis and fit to a one-site model: K_{d}'s and Bₘₐₓ's were 67.7±14.7 & 18.2±2.3 nM, and 1.09±0.12 & 2.56±0.79 fmoles/well, for A&B, respectively. Results are shown as concentration of added ligand plotted against % Bₘₐₓ. C. Effect of unlabelled soluble Aβ(1-40 and 1-42), amylin, amyloid A peptide (AA2-15) and prion peptide (PrP109-141) on the binding of ¹²⁵I-sRAGE (200 mM) to freshly prepared Aβ(1-40) immobilized on microtiter wells. Binding assays were performed as above, and the indicated concentration of unlabelled competitor was added. Data were analyzed according to a model of competitive inhibition. D. Binding of sRAGE to immobilized fibrils derived from amylin (D1), serum amyloid A peptide (2-15; D2), and prion peptide (109-141; D3). Preformed fibrils (initial monomer concentration 5 μg/well) were adsorbed to microtiter wells, and binding assays were performed as above. Binding parameters were: K_{d}'s of 68.3±5.6 (D1), 69.0±4.0 nM (D2), and 126.9±25.8 (D3). E-G. Effect of sRAGE on Aβfibrillogenesis. Aliquots of freshly prepared Aβ(1-40) dissolved in PBS were incubated at room temperature alone or with sRAGE (E&G, 1:100 molar ratio of sRAGE:Aβ; F, indicated sRAGE molar ratio), nonimmune F(ab′)_{2′} soluble polio virus receptor (sPVR) (in each case 1:100 molar ratio to Aβ) or albumin (1:100 molar ratio to Aβ). The incubation time was either varied (E) or held constant at 4 hrs (F, G), after which amyloid fibril formation was quantitated by the thioflavine T fluorescence method. In E, p<0.0001 & p<0.001 for the 1 hr and longer time points, respectively. *P<0.01. As indicated, the mean±SEM of quadruplicate determinations is shown, and experiments were repeated a minimum of three times.

FIG. 2. Domains in RAGE mediating interaction with amyloid. A. Fusion proteins of RAGE V, C or C′ domains with GST were prepared, cleaved with thrombin, and purified recombinant RAGE domains were subjected to reduced SDS-PAGE (10 μg/lane total protein; 12% gel) followed by Coomassie blue staining and N-terminal sequence analysis (note that the first five residues are the same in each case, as this sequence is derived from the vector). B. Competitive binding assays were done with preformed Aβ(1-40) fibrils (5 μg/well) adsorbed to microtiter wells, and ¹²⁵I-sRAGE (100 nM) alone or in the presence of 50-fold molar excess of unlabelled sRAGE, V (V-RAGE), C(C-RAGE) or C′ (C′-RAGE) domain. Maximal specific binding is defined as that observed in wells with ¹²⁵I-sRAGE alone minus binding in wells with ¹²⁵I-sRAGE+100-fold molar excess unlabelled sRAGE. No binding was observed in wells coated with albumin alone. C. Radioligand binding assays were performed with Aβ(1-40) fibrils (5 μg/ml) adsorbed to microtiter wells incubated with varying concentrations of ¹²⁵I-RAGE V-domain alone (total binding) or in the presence of a 100-fold molar excess of unlabelled V-domain (nonspecific binding) for 2 hrs at 37° C. Specific binding (total minus nonspecific binding), reported as a percent of Bₘₐₓ, is plotted versus added V-domain, and data was analyzed by nonlinear least squares analysis (K=78±22 nM; Bₘₐₓ=1.11±0.16 nM). D. Preformed prion peptide (PrP109-141)-, amylin- or serum amyloid A peptide (AA2-15)-derived fibrils were immobilized on microtiter plates as above (5 μg/well). Wells were incubated with either ¹²⁵I-sRAGE alone (100 nM) or in the presence of an 100-fold molar excess of unlabelled sRAGE, or unlabelled V-, C- or C′-domain. Percent inhibition of specific binding is shown. # denotes p<0.05, and * denotes p<0.01. As indicated, the mean±SEM of quadruplicate determinations is shown in panels B&D, and experiments were repeated a minimum of three times.

FIG. 3. RAGE promotes cell surface association of Aβ fibrils. A. PC12/vector (A, lane 1) or PC12/RAGE cells (A, lane 2) were analyzed by SDS-PAGE (reduced, 12% gel)/immunoblotting (A; 50 Ag/lane total protein). Migration of simultaneously run molecular weight standards is shown on the far right. B-D. PC12/RAGE cells were incubated for 4 hrs at 37° C. with preformed Aβ(1-40) fibrils (either the indicated concentration in B, or 8 μM in C&D) and nonbound material was removed by washing. As indicated, a 10-fold molar excess of sRAGE or V-domain was added (C). Cell-associated fibrils were identified by Congo red adsorption/emission (B-C) or by electron microscopy (D). The concentration of added Aβ is based on the amount of Aβ monomer initially added to the solution prior to fibril formation. In panel D, PC12/RAGE (RAGE) or PC12/vector (vector) cells were employed (upper panels) and experiments with PC12/RAGE cells (lower panels) displayed sites of RAGE expression using primary (rabbit anti-RAGE IgG) and secondary antibodies (affinity-purified goat anti-rabbit IgG conjugated to 10 nm gold particles). Arrows highlight sites of colloidal gold particles. Controls performed with preimmune rabbit IgG in place of anti-RAGE IgG or secondary antibody alone showed no specific staining pattern. Experiments were repeated a minimum of three times and the mean±SEM of triplicates is shown.

FIG. 4. Interaction of Aβ fibrils with RAGE triggers receptor-dependent activation of MAP kinases (A-C), NF-kB (D-F), and DNA fragmentation (G-I). A-B. Preformed Aβ(1-40) fibrils (125 nM) were incubated with PC12/RAGE or PC12/vector cells for the indicated times (A) or for 15 min (B1-3 utilized only PC12/RAGE cells) at 37° C. Cell lysates were subjected to SDS-PAGE (50 μg/lane total protein; reduced 10% gel)/immunoblotting using antibody to phosphorylated ERK1/2. In panels B1-B3, autoradiograms were analyzed by laser densitometry, and representative results for ERK2 from three experiments are shown. Where indicated, either anti-RAGE IgG (B1), nonimmune IgG (NI; 20 μg/ml; B1), sRAGE (10-fold molar excess compared with Aβ fibrils; B1), V-domain (10-fold molar excess; B2) or PD98059 (10 μM; B3) was added. Lanes marked medium alone contained minimal essential medium with bovine serum albumin (0.1%). C. Effect of TD-RAGE. In C1, lysates from human neuroblastoma cell cultures transiently transfected with either pcDNA3/TD-RAGE (lane 1), pcDNA3/wild-type RAGE (wt; lane 2) or pcDNA3 alone (lane 3) were subjected to SDS-PAGE (30 μg/lane protein)/immunoblotting with anti-RAGE IgG. In C2, transiently transfected cultures were incubated with preformed Aβ(1-40) fibrils (125 nM) for 15 min at 37° C. Lysates were then subjected to SDS-PAGE/immunoblotting, and densitometric analysis of the ERK2 band from three representative gels is shown. D. EMSA using ³²P-labelled consensus probe for NF-kB and nuclear extracts (10 μg/lane total protein) from stably transfected PC12 cells (D1, lane 1 shows PC12/vector and D1, lanes 2-14 & D2 show PC12/RAGE cells). Cultures were incubated with preformed Aβ(1-40) fibrils (250 nM; lanes 1-2,4-7, 9-14) for 5 hr at 37° C. alone or in the presence of anti-RAGE IgG (10 Mg/ml; D1), nonimmune IgG (10 μg/ml; D1), the indicated molar excess of sRAGE (compared with the concentration of Aβ fibrils; D1), RAGE V-domain (10-fold molar excess; D1) or PD98059 (D2). Lanes designated “cold NF-kB” indicate that an 100-fold molar excess of unlabelled NF-kB probe was added to incubation mixtures of nuclear extracts from PC12/RAGE cells treated with preformed Aβ fibrils and ³²P-labelled NF-kB probe. E. Human neuroblastoma cells were transiently transfected with either vector alone (pcDNA3; lane 1), pcDNA3/TD-RAGE (lane 2) or pcDNA3/wtRAGE (lane 3), incubated for 48 hr at 37° C., and then exposed to preformed Aβ(1-40) fibrils (250 nM) for 5 hr at 37° C. Nuclear extracts were prepared for EMSA. F. PC12/RAGE or PC12/vector cells were transiently transfected with an NF-kB-luciferase construct, and 48 hrs later cultures were exposed to preformed Aβ(1-40) fibrils (500 nM) for 6 hrs at 37° C. followed by harvest and determination of luciferase activity. Where indicated, anti-RAGE IgG (10 pg/ml), nonimmune IgG (10 μg/ml) or PD98059 (25 μM) was added. G. PC12/RAGE or PC12/vector cells were incubated with preformed Aβ(1-40) fibrils at the indicated concentration (G1) or PC12/RAGE cells were exposed to Aβ fibrils (1 μM in G2 and 2 μM in G3) for 20 hrs at 37° C. alone or in the presence of anti-RAGE IgG (50 μg/ml; G2), nonimmune IgG (NI; 50 μg/ml; G2), PD98059 (25 μM) (G2) or an 10-fold molar excess of sRAGE (G3). Samples were harvested to determine cytoplasmic histone-associated DNA fragments. H. TUNEL staining of nuclei from representative fields of PC12/vector (H1-2) and PC12/RAGE cells (H3-4) incubated in medium alone (H1,3) or with preformed Aβ(1-40) fibrils (1 μM; H2,4) for 20 hrs at 37° C. H5 shows quantitation of TUNEL results reported as % TUNEL positive nuclei per high power field divided by the total number of nuclei in the same fields. In each case, 7 fields from three representative experiments were analyzed. I. Neuroblastoma cells were transiently transfected with either pcDNA3 alone, pcDNA3/TD-RAGE or pcDNA3/wtRAGE, and incubated for 48 hrs at 37° C. Preformed Aβ(1-40) fibrils (2 μM) were added for another 12 hrs at 37° C., and cultures were then harvested for determination of DNA fragmentation as in A. *P<0.01. Experiments were repeated a minimum of three times and the mean±SEM of triplicate determinations is shown.

FIG. 5. Interaction of prion peptide-derived and amylin fibrils with cell surface RAGE. A. PC12/RAGE or PC12/vector cells were incubated with prion peptide (5 μg/ml) or amylin fibrils (5.6 μg/ml; concentrations refer to that of the monomer initially added) for 4 hrs at 37° C. Unbound material was removed by washing, Congo red was added and dye binding was determined by Congo red adsorption/emission. B-C. EMSA for NF-kB with amylin (B) or prion peptide (C) fibrils incubated with transfected PC12 cells. PC12/RAGE (B, lanes 2-4&9-14 and C, lanes 2-10) or PC12/vector cells (B, lanes 5-7 and C, lane 1) were incubated with preformed amylin (concentration as indicated) and prion peptide (1 μM) fibrils for 5 hrs at 37° C. Nuclear extracts (10 μg protein) were prepared and incubated with ³²P-labelled consensus NF-kB probe alone or in the presence of an 100-fold excess of unlabelled NF-kB probe (cold NF-kB). Where indicated, either sRAGE (5-fold molar excess), anti-RAGE IgG (10 μg/ml) or nonimmune IgG (NI; 10 μg/ml) was added. D. PC12/vector (D1 as indicated) or PC12/RAGE cells (D1 as indicated, D2 & D3) were incubated with prion peptide-derived fibrils (1 μM) for 20 hrs at 37° C., cultures were harvested and the ELISA for DNA fragmentation was performed. As shown, anti-RAGE IgG (50 μg/ml; D2), nonimmune IgG (NI; 50 μg/ml; D2), or sRAGE (10-fold molar excess; D3) were also added. E. Human neuroblastoma cells were transfected with pcDNA3 alone, pcDNA3/wtRAGE or pcDNA3/TD-RAGE using lipofectamine plus, incubated for 48 hrs, and then exposed to prion fibrils (PrP; 3 μM) for 12 hrs. DNA fragmentation was determined by ELISA. *p<0.01 and #p<0.05. The mean±SEM of quadruplicate determination is shown, and experiments were repeated a minimum of three times.

FIG. 6. Interaction of RAGE with amyloid A fibrils. A-B. Microtiter plates were incubated with Aβ(1-40), apoSAA1, apoSAA2, apoSAAce/j, apoA-I or apoA-II, amyloid A fibrils (AA)(5 μg/well in each case), and a binding assay was performed with ¹²⁵I-sRAGE (100 nM) alone or in the presence of 100-fold excess unlabelled sRAGE (as indicated, +sRAGE). For other experiments (B), binding assays were performed as above with immobilized Aβ, amyloid A fibrils or SAA2 adsorbed to the microtiter wells, and ¹²⁵I-sRAGE (100 nM) in the presence/absence of anti-RAGE IgG (10 μg/ml) (nonimmune IgG was without effect; not shown). C. ApoSAA2 (SAA2), amyloid A (AA) fibrils, or ApoSAA1 (SAA1) was adsorbed to microtiter wells (5 μg/well in each case) and binding assays were performed with the indicated concentrations of ¹²⁵I-sRAGE alone (total binding) or in the presence of an 50-fold molar excess of unlabelled sRAGE (nonspecific binding). Specific binding is shown, and data was analyzed by nonlinear least squares analysis; K_{d}=72.8±16.3 nM (SAA2) and 60.3±12.5 nM (amyloid A). No saturable binding was observed for SAA1. D. Amyloid A fibrils (initial monomer concentration as indicated) were incubated with either PC12/vector (vector) or PC12/RAGE (RAGE) cells for 4 hrs at 37° C. Unbound material was removed by washing, Congo red was added for 30 min, and bound dye was determined by Congo red emission/adsorption. E. Interaction of amyloid A fibrils with PC12/RAGE cells causes NF-kB activation. PC12/vector (lane 1) or PC12/RAGE (lanes 2, 4-8) cells were incubated with amyloid A fibrils (100 nM) for 5 hrs at 37° C. Nuclear extracts were analyzed by EMSA with ³²P-labelled NF-kB consensus probe (10 μg protein/lane). Where indicated, anti-RAGE IgG (5 μg/ml) or nonimmune IgG (NI; 5 μg/ml) was added during incubation of fibrils with cells. The lane designated “cold NF-kB” indicates the presence of an 100-fold excess of unlabelled probe added to nuclear extracts of amyloid A-treated PC12/RAGE cells during their incubation with ³²P-labelled NF-kB probe. *p<0.01 and #p<0.05. The mean±SEM is shown as indicated, and experiments were repeated a minimum of three times.

FIG. 7. Effect of sRAGE on systemic amyloidosis in a murine model. A. SAA in mouse plasma was assessed on day 5 in each experimental group: control, control+sRAGE (200 μg), AEF/SN+vehicle, and AEF/SN+sRAGE (200 μg) (see text for experimental protocol). Samples were subjected to SDS-PAGE (reduced 5-20% gel)/immunoblotting with rabbit anti-apoSAA IgG (1 μg/ml). Migration of simultaneously run molecular weight standards (designated in kilodaltons) is shown on the left of the gel. B. Nuclear extracts were prepared from spleens following induction of amyloid with AEF/SN using animals treated with sRAGE or vehicle (day 5). EMSA was performed with ³²P-labelled NF-kB probe and the following samples (10 μg protein/lane): lanes 1-2, control spleens from noninjected animals (saline-injected controls were identical); lanes 3-4, after 5 days of AEF/SN+vehicle, mouse serum albumin (200 μg/animal); lanes 5-6, after 5 days of AEF/SN+20 μg/animal of sRAGE/day; lanes 7-8, after 5 days of AEF/SN+100 Ag/animal of sRAGE/day; lane 9, 100-fold excess unlabelled NF-kB probe added to sample 3 during incubation with ³²P-labelled probe; and lane 10, HeLa nuclear extract. Results from two representative animals in each group are shown. C. Northern analysis for IL-6 (C1) and HO-1 (C1), and M-CSF (C₂₋₃) transcripts in the spleen, and densitometry (C4). As indicated, representative samples from 3 or 5 animals in each group are shown. Total RNA harvested from spleens of control mice or those treated with AEF/SN+vehicle or AEF/SN+sRAGE (day 5; 100 μg/day of sRAGE unless indicated otherwise, as in C3) was subjected to Northern analysis (20 μg/lane) using probes for murine IL-6 (C1), HO-1 (C1), or M-CSF (C₂₋₃). In panel 1, ethidium bromide staining displays ribosomal RNA as a control for loading of RNA from AEF/SN groups (this was done for each group in all experiments, and loading was found to be equivalent, but is only shown for the AEF/SN group in panel 1). In C3, mice were treated with the indicated concentration of sRAGE once daily, total RNA was prepared on day 5 and Northern blots were hybridized with ³²P-labelled M-CSF probe (results from a representative mouse in each group are shown). In C4, densitometic analysis of Northerns is shown from control, AEF/SN and AEF/SN+sRAGE (200 pg/day) groups (day 5; N=5/group). D-E. Immunostaining for IL-6 (D) and M-CSF (E) in splenic tissue (day 5): panel 1, control mouse; panel 2, after 5 days of AEF/SN+vehicle; panel 3, after 5 days of AEF/SN+sRAGE (100 μg/day); and panel 4, image analysis of data from splenic tissue of the same animal groups shown in panels 1-3 using the Universal Imaging System. F. C57BL6 mice treated with AEF/SN in the presence/absence of sRAGE at the indicated daily dose were analyzed for amyloid burden in the spleen after 5 days. G. Northern blotting of RAGE transcripts in total RNA (20 pg/lane) isolated on day 5 from spleens (G1) of AEF/SN+sRAGE mice (100 μg; lanes 1-2), control mice (lanes 3-4), or AEF/SN+vehicle mice (lanes 5-6). Blots were hybridized with ³²P-labelled mouse RAGE cDNA (equivalent RNA loading was confirmed by ethidium bromide staining of ribosomal RNA bands; not shown). G2 shows densitometric analysis of blots from animals treated as in G1. H. Immunostaining for RAGE was performed on splenic tissue from control mice (H1), AEF/SN+vehicle mice (H2), and AEF/SN+sRAGE mice (H3; 100 μg)(day 5 in each case). Panel H4 shows image analysis of samples under the same conditions as in H1-3. H5-6 shows immunostaining for SAA in spleens of control and AEF/SN mice, respectively. I. Immunoprecipitation of sRAGE/SAA complex in mouse plasma. Plasma from C57BL6 mice (50 μl/animal) treated with AEF/SN+vehicle or AEF/SN+sRAGE (100 μg; day 5) was immunoprecipitated with anti-apoSAA IgG (5 μg/ml), anti-RAGE IgG (5 μg) or IgG from preimmune serum (5 μg/ml) followed by SDS-PAGE/immunoblotting with anti-apoSAA IgG (1 μg/ml; reduced 5-20% gel; 7I1) or anti-RAGE IgG (1 μg/ml; reduced 10% gel; 7I2). Panel 1: lane 1, immunoprecipitation of plasma from AEF/SN+sRAGE mice with anti-RAGE IgG followed by immunoblotting with anti-apoSAA IgG; lane 2, immunoprecipitation of plasma from AEF/SN+sRAGE mice with preimmune IgG followed by immunoblotting with anti-apoSAA IgG; and, lane 3, immunoblotting of AEF/SN plasma with anti-apoSAA IgG. Panel 2: lane 1, immunoprecipitation of plasma from AEF/SN+sRAGE mice with anti-apoSAA IgG followed by immunoblotting with anti-RAGE IgG; lane 2, immunoprecipitation of plasma from AEF/SN+sRAGE mice with preimmune IgG followed by immunoblotting with anti-RAGE IgG; and, lane 3, immunoblotting of purified sRAGE (1 μg). Immunoprecipitation of plasma from AEF/SN mice not treated with sRAGE showed no detectable sRAGE and no evidence of SAA-sRAGE complex. * indicates p<0.01. Studies were repeated a minimum of three times, and there were five animals in experimental groups. Magnification: D x80; E x280; H x80.

FIG. 8. Dissociation constants for the interaction of RAGE with several peptides in solution evaluated by fluorescence FIG. 9. Expression of RAGE, deposition of amyloid A and expression of M-CSF in human spleen. (a-e), Sections from a patient with systemic reactive amyloidosis (amyloid A), immunostained with antibody against RAGE (a), or amyloid A (b and inset of b), double-stained with antibodies against RAGE (c), and CD14 (d; to indentify mononuclear phagocytes), or stained with antibody against M-CSF (9e). f and g, Tissue from an age-matched control, stained with antibody against RAGE (e) or M-CSF (f). Scale bars represent 10 μm (a, b, f), 2 μm (c, d), and 4 μm (d, g).

FIG. 10. Interaction of RAGE with amyloid A fibrils, and RAGE-dependent activation of BV-2 transformed mononuclear phagocytes by SAA1.1. (a), Microtiter plates were incubated with synthetic amyloid β-protein 1-40 (Aβ) or purified SAA2.1, SAA1.1, SAA2.2, AI, AII or amyloid A (AA) fibrils (5 μg/well for each; ‘Coating’). Binding assays used 100 nM¹²⁵I-sRAGE alone (−) or in the presence of a 50-fold excess of unlabeled sRAGE (+). (b), Binding assays with immobilized amyloid protein (Aβ), amyloid A fibrils (AA) or SAA1.1 adsorbed to microtiter wells, and 100 nM ¹²⁵I-sRAGE in the presence or absence of 10 μg/ml IgG antibody against RAGE (a-RAGE)(nonimmune IgG had no effect; data not shown). A and b, Data represent mean±s.e.m. of quadruplicate determinations from three separate experiments; p<0.01. C, SAA1.1, amyloid A (AA) fibrils or SAA2.1 was adsorbed to microtiter wells (5 μg/well for each); binding assay used ¹²⁵I-sRAGE alone (total binding) or in the presence of a 50-fold molar excess of unlabeled sRAGE (nonspecific binding). Data represent % maximum specific binding (total minus nonspecific binding/maximal specific binding), and were analyzed by nonlinear least-squares analysis. K₄=72.8±16.3nM and Bₘₐₓ=2.4±0.4 fmol/well, SAA1.1; and 60.3±12.5 nM and Bₘₐₓ=2.7±0.5 fmol/well, amyloid A. There is no saturable binding for SAA2.1 (lane 2). Nuclear extracts were analyzed by EMSA (10 μg total protein/lane) with a ³⁷P-labeled consensus oligonucleotide probed for NF-κB. Cultures were pre-incubated with 10 μg/ml antibody against RAGE (ab′)₂(lane 3) or nonimmune F9(ab′)₂(lane 4), followed by exposure of cells to serum-free medium with 300 nM fibrillar SAA1.1 A 100-fold excess of unlabeled NF-κB probe was added to nuclear extracts from BV-2 cells exposed to SAA1.1 (lane 5) Duplicate cultures of BV-2 cells were transfected with pcDNA3-DN-RAGE (lanes 6 and 7) or vector alone (pcDNA3; lanes 8 and 9); and then incubated in serum-free medium with 300 nM SAA1.1. Nuclear extracts were analyzed EMSA with the NF-κB probe. e and f, The incubation of SAA1.1 with BV-2 cells was continued for 24 h. Treatment included incubation in medium alone (lane 1), with SAA1.1 (lane 2), with antibody against RAGE F(ab′)₂, and then SAA1.1 (lane 3), or with nonimmune F(ab′)₂ and then SAA1.1 (lane 4). Total RNA was assessed by northern blot analysis using ³²P-labeled cDNA probes for HO-1 (e) or M-CSF (f). 18S, Ethidium bromide staining shows ribosomal RNA as a control for loading of RNA.

FIG. 11. Effect of RAGE blockade on systemic amyloidosis in a mouse model: plasma SAA levels, splenic NF-κB activation and expression of transcripts for cell stress markers. (a), SAA in mouse plasma was assessed on day 5 (treatment, below blot), by reduced 5-20% SDS-PAGE and immunoblotting with 1 μg/ml rabbit antibody against SAA IgG. Left margin, migration of molecular weight standards (in kilodaltons). (b), Nuclear extracts prepared from spleens after induction of amyloid with AEF-SN using mice treated with sRAGE or vehicle (day 5) were analyzed by EMSA used ³²P-labeled NF-κB probe (10 μg protein/lane). Lanes 1 and 2, control (noninjected mice; saline-injected controls were identical); lanes 3 and 4, AEF/SN plus vehicle (200 μg mouse serum albumin/mouse); lanes 5 and 6, AEF/SN plus 20 μg sRAGE/mouse per day; lanes 7 and 8, AEF/SN plus 100 μg sRAGE/mouse per day; lane 9, 100-fold excess unlabeled ³²P-labeled probe; lane 10, HeLa nuclear extract (positive control). Data represent two mice in each group. (c) Amyloid was induced with AEF/SN using mice treated with sRAGE or vehicle; mice received either antibody against RAGE f(ab′)₂(α-RAGE) or nonimmune F(ab′)₂(NI) (100 μg/mouse for each) 1 day before and on days 1-4 of AEF/SN treatment. Nuclear extracts prepared from spleens (day 5) were analyzed by EMSA using ³²P-labeled NF-κB probe (10 μg protein/lane). Lanes 1-3, control mice (no AEF/SN); Lanes 4-6, mice given AEF/SN; additional treatments below gel (a-RAGE, antibody against RAGE F(ab′)₂; NI, nonimmune F(ab′)₂). (d-g), Total RNA from spleens of control mice or mice treated with AEF/SN plus vehicle or AEF/SN plus sRAGE (day 5; sRAGE dose/day: 100 μg, d and e; along horizontal axis, (f) was assessed by northern blot analysis (20 μg/lane) using probes for mouse IL-6 or HO-1 (d) or M-CSF (e and f). Data represent three (e) or five (f) mice in each group. (d) (third row), Ethidium bromide staining shows ribosomal RNA as a control for loading of RNA from groups of mice treated with AEF/SN (loading was equivalent for all groups in all experiments, but is only shown for the group treated with AEF/SN in d). (f), ³²P-labeled M-CSF probe. Data represent one mouse of each group. (g), Densitometric analysis of northern blots (treatments, below graph; n=5 per group), and of experiments in which mice treated with AEF/SN received either 100 μg/ml antibody against RAGE F9(ab′)₂ or 100 μg/ml NI F(ab′)₂(n=5 per group).

FIG. 12. Effect of RAGE blockade on systemic amyloidosis in a mouse model. Expression of IL-6 (a-e) and M-CSF) (f-j) in splenic tissue (day 5), by immunostaining (a-c and f-h) and image analysis (d, e, i, j). Mouse treatments: a and f, Control: b and g, AEF/SN plus vehicle; c and h, AEF/SN plus 100 μg sRAGE/day. d and j, image analysis of data in a-c and f-h. e and j, image analysis (day 5) of experiments in which mice treated with AEF/SN received either antibody against RAGE F(ab′)₂(α-RAGE) or nonimmune F(ab′)₂(NI) (100 μg for each). n=5 mice per group. Original magnification, x80(a-c) and x280(f-h)*, P<0.01.

FIG. 13. Soluble RAGE infusion in a mouse model of systemic amyloidosis: effect on splenic RAGE expression. a and b, Northern blot (a) and densitometric (b) analysis of RAGE transcripts in total RNA (20 μg/lane) isolated on day 5 from spleens of mice treated with AEF/SN plus 100 μg sRAGE (lanes 1 and 2), control mice (lanes 3 and 4) or mice treated with AEF/SN plus vehicle (lanes 5 and 6). Blots were hybridized with ³²P-labeled mouse RAGE cDNA (equivalent RNA loading confirmed by ethidium bromide staining of ribosomal RNA bands; not shown). *, P<0.01. (c-e), immunostaining for RAGE, on splenic tissue from a control mouse (c) and mice treated with AEF/SN plus vehicle (d) or plus 100 μg sRAGE (e) (day 5). f and g, immunostaining for SAA in spleens of a control mouse (f) and a mouse treated with AEF/SN (g). Original magnification (c-g), x80. h, image analysis for the intensity of RAGE staining (arbitrary units) for c-e; treatment, below graph. *, p<0.01. n=5 mice per group.

FIG. 14. Effect of RAGE blockade in a mouse model of systemic amyloidosis: isolation of SAA-sRAGE complex from mouse plasma and effect on splenic amyloid deposition. a and b, Immunoprecipitation of sRAGE-SAA complex in mouse plasma. Plasma from CS7BI/6 mice (50 μl/mouse; day 5) was immunoprecipitated, separated by SDS-PAGE and immunoblotted. Treatment; immunoprecipitation antibody; blot antibody: a, Lane 1, AEF/SN plus 100 μg sRAGE; RAGE; SAA; lane 2, AEF/SN plus 100 μg sRAGE; preimmune 100 μg; SAA; lane 3, AEF/SN plus vehicle (5 μg HDL protein from mouse); 100 μg; SAA. b, Lane 1, AEF/SN plus sRAGE; SAA; RAGE; lane 2, AEF/SN plus sRAGE, preimmune; RAGE; lane 3, immunoblot of 1 μg purified sRAGE; none; RAGE immunoprecipitation of plasma from mice given AEF/SN not treated with sRAGE showed no detectable sRAGE and no evidence of the SAA-sRAGE complex. *, p<0.01. Studies were repeated a minimum of three times (n=5 mice per group.) c and d, C57BI/6 mice were treated with AEF/SN and sRAGE (c, horizontal axis), or with antibody against RAGE F(ab′)₂ (d; a-RAGE; dose, horizontal axis) or 100 μg nonimmune F(ab′)₂(d; NI); the amyloid burden in the spleen was determined after 5d. Control, untreated mouse spleen. n=mice per group. P values, above bars.

FIG. 15. Amylin and prion-peptide-derived fibrils bind RAGE and mediate RAGE-dependent NF-κB activation on BV-2 cells. a and b, Human anylin fibrils (a; initial monomer concentration, about 5 μg/ml) or prion-peptide-derived fibrils (b; about 5 μg/ml) were adsorbed to microtiter plates; after blockade with albumin and incubation with ¹²⁵I-sRAGE alone or in the presence of a 20-fold excess of unlabeled sRAGE, bound ¹²⁵I-sRAGE was determined. Data represent % maximum specific binding (% Bₘₐₓ; total minus nonspecific binding/maximum specific binding) versus added ligand. Data were analyzed by nonlinear least-squares analysis and fit to a one-site model (Bₘₐₓ=21.9±4.8 and 111±26.7 fmol/well for sRAGE binding to amylin and prion peptide-derived fibrils, respectively). c and d, Competitive binding studies. Wells were coated with either amylin fibrils (c) or prion-peptide-derived fibrils (d) and incubated with 40 nM ¹²⁵I-sRAGE alone or in the presence of a 20-fold molar excess of soluble prion peptide (random configuration), soluble amylin peptide (random configuration), priion peptide-derived fibrils (prion fibril), amylin fibrils or erabutoxin B. Maximum specific binding (100%) was defined as the difference of total binding (with ¹²⁵I-sRAGE alone) minus nonspecific binding (with ¹²⁵I-sRAGE plus a 20-fold excess of unlabeled sRAGE). *, p<0.01. e and f, RAGE-dependent NF-κB activation in BV-2 cells incubated with medium alone (0; e, lane 1 and f, lane 2) or 4 μg/ml amylin fibrils (e, lanes 2-5) or prion peptide-derived fibrils (f, lanes 3-6); some cultures were preincubated with 10 μg/ml antibody against RAGE f(ab′)₂(e, lane 3 and f, lane 4), or nonimmune F(ab′)₂(e, lane 4 and f, lane 5) before exposure to fibrils, and some had a 100-fold excess of unlabeled NF-κB probe added (e, lane 5 and f, lane 6). FP (f, lane 1), migration of free probe alone. Nuclear extracts were analyzed by EMSA (10 μg total protein/lane) with ³²P-labeled consensus oligonucleotide probe for NF-κB.

### DETAILED DESCRIPTION OF THE INVENTION

Abbreviations: Aβ, amyloid β-peptide; AD, Alzheimer's disease; AEF/SN, amyloid enhancing factor/silver nitrate; AGE, advanced glycation endproducts; βAPP, β-amyloid precursor protein; EMSA, electrophoretic mobility shift assay; HO-1, heme oxygenase type 1; IL, interleukin; ERK, Extracellular signal-regulated protein kinase; GST, glutathione-S-transferase; MAP kinase, mitogen-activated protein kinase; M-CSF, monocyte-colony stimulating factor; MEK, mitogen-activated protein kinase; NF-kB, nuclear factor kB; SAA, serum amyloid A; sRAGE, soluble RAGE; RAGE, receptor for AGE; TD, tail-deletion; wt, wild-type.

This invention provides a method of inhibiting the binding of a β-sheet fibril to RAGE on the surface of a cell which comprises contacting the cell with a binding inhibiting amount of a compound capable of inhibiting binding of the β-sheet fibril to RAGE so as to thereby inhibit binding of the β-sheet fibril to RAGE.

In one embodiment, the β-sheet fibril is amyloid fibril. In another embodiment, the β-sheet fibril is a prion-derived fibril. The β-sheet fibril can comprise amyloid-β peptide, amylin, amyloid A, prion-derived peptide, transthyretin, cystatin C, gelsolin or a peptide capable of forming amyloid. In one embodiment, the β-sheet fibril is an amyloid-β peptide which comprises Aβ (1-39), Aβ (1-40), Aβ(1-42) or Aβ (1-40) Dutch variant.

In one embodiment, the above compound is sRAGE or a fragment thereof. In another embodiment, the compound is an anti-RAGE antibody or portion thereof. In one embodiment, the antibody is a monoclonal antibody. In one embodiment, the monoclonal antibody is a human, a humanized, or a chimeric antibody. In one embodiment, the above compound comprises a Fab fragment of an anti-RAGE antibody. In one embodiment, the Fab fragment is a F(ab′)₂ fragment. In one embodiment, the above compound comprises the variable domain of an anti-RAGE antibody. In one embodiment, the above compound comprises one or more CDR portions of an anti-RAGE antibody. In one embodiment, the antibody is an IgG antibody.

In one embodiment, the compound comprises a peptide, polypeptide, peptidomimetic, a nucleic acid, or an organic compound with a molecular weight less than 500 daltons. The polypeptide may be a peptide, a peptidomimetic, a synthetic polypeptide, a derivative of a natural polypeptide, a modified polypeptide, a labelled polypeptide, a polypeptide which includes non-natural peptides, a nucleic acid molecule, a small molecule, an organic compound, an inorganic compound, or an antibody or a fragment thereof. The peptidomimetic may be identified from screening large libraries of different compounds which are peptidomimetics to determine a compound which is capable of preventing accelerated atherosclerosis in a subject predisposed thereto. The polypeptide may be a non-natural polypeptide which has chirality not found in nature, i.e. D-amino acids or L-amino acids.

The compound may be the isolated peptide having an amino acid sequence corresponding to the amino acid sequence of a V-domain of RAGE. The compound may be any of the compounds or compositions described herein.

The compound may be a soluble V-domain of RAGE. The compound may comprise an antibody or fragment thereof. The antibody may be capable of specifically binding to RAGE. The antibody may be a monoclonal antibody or a polyclonal antibody or a fragment of an antibody. The antibody fragment may comprise a Fab or Fc fragment. The fragment of the antibody may comprise a complementarity determining region.

In one embodiment, the compound is capable of specifically binding to the β-sheet fibril. In one embodiment, the compound is capable of specifically binding to RAGE.

In one embodiment, the compound is an antagonist, wherein the antagonist is capable of binding the RAGE with higher affinity than AGEs, thus competing away the effects of AGE's binding.

In another embodiment, the compound is a ribozyme which is capable of inhibiting expression of RAGE. In another embodiment, the compound is an anti-RAGE antibody, an anti-AGE antibody, an anti-V-domain of RAGE antibody. The antibody may be monoclonal, polyclonal, chimeric, humanized, primatized. The compound may be a fragment of such antibody.

In one embodiment, the antibody may be capable of specifically binding to RAGE. The antibody may be a monoclonal antibody, a polyclonal antibody. The portion or fragment of the antibody may comprise a F_{ab} fragment or a F_{c} fragment. The portion or fragment of the antibody may comprise a complementarity determining region or a variable region.

In one embodiment, the peptide is an advanced glycation endproduct (AGE) or fragment thereof. In another embodiment, the peptide is a carboxymethyl-modified peptide. For example, peptide may be a carboxymethyl-lysine-modified AGE. In another embodiment, the peptide is a synthetic peptide.

As used herein “RAGE or a fragment thereof” encompasses a peptide which has the full amino acid sequence of RAGE as shown in Neeper et al. (1992) or a portion of that amino acid sequence. The “fragment” of RAGE is at least 5 amino acids in length, preferably more than 7 amino acids in length, but is less than the full length shown in Neeper et al. (1992). In one embodiment, the fragment of RAGE comprises the V-domain, which is a 120 amino acid domain depicted in Neeper et al. (1992). For example, the fragment of RAGE may have the amino acid sequence of the V-domain sequence of RAGE.

In another embodiment, the compound has a net negative charge or a net positive charge. In a further embodiment, the compound comprises a fragment of naturally occurring soluble receptor for advanced glycation endproduct (sRAGE).

The compound identified by the screening method may comprise a variety of types of compounds. For example, in one embodiment the compound is a peptidomimetic. In another embodiment, the compound is an organic molecule. In a further embodiment, the compound is a polypeptide, a nucleic acid, or an inorganic chemical. Further, the compound is a molecule of less than 10,000 daltons. In another embodiment, the compound is an antibody or a fragment thereof. The antibody may be a polyclonal or monoclonal antibody. Furthermore, the antibody may be humanized, chimeric or primatized. In another embodiment, compound is a mutated AGE or fragment thereof or a mutated RAGE or a fragment thereof.

The compound may be an sRAGE polypeptide such as a polypeptide analog of sRAGE. Such analogs include fragments of sRAGE. Following the procedures of the published application by Alton et al. (WO 83/04053), one can readily design and manufacture genes coding for microbial expression of polypeptides having primary conformations which differ from that herein specified for in terms of the identity or location of one or more residues (e.g., substitutions, terminal and intermediate additions and deletions). Alternately, modifications of cDNA and genomic genes can be readily accomplished by well-known site-directed mutagenesis techniques and employed to generate analogs and derivatives of sRAGE polypeptide. Such products share at least one of the biological properties of sRAGE but may differ in others. As examples, products of the invention include those which are foreshortened by e.g., deletions; or those which are more stable to hydrolysis (and, therefore, may have more pronounced or longerlasting effects than naturally-occurring); or which have been altered to delete or to add one or more potential sites for O-glycosylation and/or N-glycosylation or which have one or more cysteine residues deleted or replaced by e.g., alanine or serine residues and are potentially more easily isolated in active form from microbial systems; or which have one or more tyrosine residues replaced by phenylalanine and bind more or less readily to target proteins or to receptors on target cells. Also comprehended are polypeptide fragments duplicating only a part of the continuous amino acid sequence or secondary conformations within sRAGE, which fragments may possess one property of sRAGE and not others. It is noteworthy that activity is not necessary for any one or more of the polypeptides of the invention to have therapeutic utility or utility in other contexts, such as in assays of sRAGE antagonism. Competitive antagonists may be quite useful in, for example, cases of overproduction of sRAGE.

Of applicability to peptide analogs of the invention are reports of the immunological property of synthetic peptides which substantially duplicate the amino acid sequence existent in naturally-occurring proteins, glycoproteins and nucleoproteins. More specifically, relatively low molecular weight polypeptides have been shown to participate in immune reactions which are similar in duration and extent to the immune reactions of physiologically-significant proteins such as viral antigens, polypeptide hormones, and the like. Included among the immune reactions of such polypeptides is the provocation of the formation of specific antibodies in immunologically-active animals (Lerner et al., Cell, 23, 309-310 (1981); Ross et al., Nature, 294, 654-658 (1981); Walter et al., Proc. Natl. Acad. Sci. USA, 78, 4882-4886 (1981); Wong et al., Proc. Natl. Sci. USA, 79, 5322-5326 (1982); Baron et al., Cell, 28, 395-404 (1982); Dressman et al., Nature, 295, 185-160 (1982); and Lerner, Scientific American, 248, 66-74 (1983). See also, Kaiser et al. [Science, 223, 249-255 (1984)] relating to biological and immunological properties of synthetic peptides which approximately share secondary structures of peptide hormones but may not share their primary structural conformation. The compounds of the present invention may be a peptidomimetic compound which may be at least partially unnatural. The peptidomimetic compound may be a small molecule mimic of a portion of the amino acid sequence of sRAGE. The compound may have increased stability, efficacy, potency and bioavailability by virtue of the mimic. Further, the compound may have decreased toxicity. The peptidomimetic compound may have enhanced mucosal intestinal permeability. The compound may be synthetically prepared. The compound of the present invention may include L-,D- or unnatural amino acids, alpha, alpha-disubstituted amino acids, N-alkyl amino acids, lactic acid (an isoelectronic analog of alanine). The peptide backbone of the compound may have at least one bond replaced with PSI-[CH═CH] (Kempf et al. 1991). The compound may further include trifluorotyrosine, p-Cl-phenylalanine, p-Br-phenylalanine, poly-L-propargylglycine, poly-D,L-allyl glycine, or poly-L-allyl glycine.

One embodiment of the present invention is a peptidomimetic compound wherein the compound has a bond, a peptide backbone or an amino acid component replaced with a suitable mimic. Examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, cysteine (acetamindomethyl), N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, Boc-L-thioproline. (Blondelle, et al. 1994; Pinilla, et al. 1995).

In one embodiment, the compound is a peptide wherein the free amino groups have been inactivated by derivitization. For example, the peptide may be an aryl derivative, an alkyl derivative or an anhydride derivative. The peptide may be acetylated. The peptide is derivatized so as to neutralize its net charge. As used herein “inactivated by derivatization” encompasses a chemical modification of a peptide so as to cause amino groups of the peptide to be less reactive with the chemical modification than without such chemical modification. Examples, of such chemical modification includes making an aryl derivative of the peptide or an alkyl derivative of the peptide. Other derivatives encompass an acetyl derivative, a propyl derivative, an isopropyl derivative, a buytl derivative, an isobutyl derivative, a carboxymethyl derivative, a benzoyl derivative. Other derivatives would be known to one of skill in the art.

In another embodiment, the compound may be soluble RAGE (sRAGE) or a fragment thereof. Soluble RAGE is not located on the cell surface and is not associated with a cell membrane. Soluble RAGE (sRAGE) is the RAGE protein free from the cell membrane. For example, sRAGE is not imbedded in the cell surface. In one embodiment, sRAGE comprises the extracellular two-thirds of the amino acid sequence of membrane-bound RAGE.

In another embodiment, the compound is an anti-RAGE antibody or fragment thereof. In another embodiment, the compound is an sRAGE peptide. In another embodiment, the compound consists essentially of the ligand binding domain of sRAGE peptide. In another embodiment, the compound is a nucleic acid molecule or a peptide. In another embodiment, the nucleic acid molecule is a ribozyme or an antisense nucleic acid molecule.

In one embodiment, the cell is present in a tissue. In one embodiment, the tissue is a spleen. The tissue can encompass other types of tissues not mentioned herein.

In one embodiment, the inhibition of binding of the β-sheet fibril to RAGE has the consequence of decreasing the load of β-sheet fibril in the tissue.

In one embodiment, the cell is a neuronal cell, an endothelial cell, a glial cell, a microglial cell, a smooth muscle cell, a somatic cell, a bone marrow cell, a liver cell, an intestinal cell, a germ cell, a myocyte, a mononuclear phagocyte, an endothelial cell, a tumor cell, or a stem cell. The cell may also be another kind of cells not explicitly listed herein. The cell may be any human cell.

The cell may be a normal cell, an activated cell, a neoplastic cell, a diseased cell or an infected cell. The cell may also be a RAGE-transfected cell. The cell may also be a cell which expresses RAGE.

The peptides or antibodies of the present invention may be human, mouse, rat or bovine.

In the embodiments wherein the compound is, for example, a protein or antibody, the amino acids of the proteins and peptides of the subject invention may be replaced by a synthetic amino acid which is altered so as to increase the half-life of the peptide or to increase the potency of the peptide, or to increase the bioavailability of the peptide.

In one embodiment, the inhibition of binding of the β-sheet fibril to RAGE has the consequence of inhibiting fibril-induced programmed cell death.

As used herein, “programmed cell death” involves activation of enzymes such as caspases, and fragmentation of nuclear DNA.

In one embodiment, the inhibition of binding of the β-sheet fibril to RAGE has the consequence of inhibiting fibril-induced cell stress. In one embodiment, the inhibition of fibril-induced cell stress is associated with a decrease in expression of macrophage colony stimulating factor. In another embodiment, the inhibition of fibril-induced cell stress is associated with a decrease in expression of interleukin-6. In another embodiment, the inhibition of fibril-induced cell stress is associated with a decrease in expression of heme oxygenase type 1.

As used herein, the term “cell stress” involves the increased expression of interleukin-6 (IL-6), macrophage colony stimulating factor (M-CSF), heme oxygenase type 1 (HO-1), activation of MAP kinases, and activation of the transcription factor NF-κB. It encompasses the perturbation of the ability of a cell to ameliorate the toxic effects of oxidants. Oxidants may include hydrogen peroxide or oxygen radicals that are capable of reacting with bases in the cell including DNA. A cell under “oxidant stress” may undergo biochemical, metabolic, physiological and/or chemical modifications to counter the introduction of such oxidants. Such modifications may include lipid peroxidation, NF-kB activation, heme oxygenase type I induction and DNA mutagenesis. Also, antioxidants such as glutathione are capable of lowering the effects of oxidants. The present invention provides agents and pharmaceutical compositions which are capable of inhibiting the effects of oxidant stress upon a cell. The invention also provides methods for ameliorating the symptoms of oxidant stress in a subject which comprises administering to the subject an amount of the agent or pharmaceutical composition effective to inhibit oxidant stress and thereby ameliorate the symptoms of oxidant stress in the subject.

In one embodiment, the cell is present in a subject and the contacting is effected by administering the compound to the subject.

The subject may be a mammal or non-mammal. The subject may be a human, a primate, an equine subject, an opine subject, an avian subject, a bovine subject, a porcine, a canine, a feline or a murine subject. In another embodiment, the subject is a vertebrate. The subject may be a human, a primate, an equine subject, an opine subject, a mouse, a rat, a cow, an avian subject, a bovine subject, a porcine, a canine, a feline or a murine subject. In a preferred embodiment, the mammal is a human being. The subject may be a diabetic subject. The subject may be suffering from an apolipoprotein deficiency, or from hyperlipidemia. The hyperlipidemia may be hypercholesterolemia or hypertriglyceridemia. The subject may have a glucose metabolism disorder. The subject may be an obese subject. The subject may have genetically-mediated or diet-induced hyperlipidemia. AGEs form in lipid-enriched environments even in euglycemia. The subject may be suffering from oxidant stress. The subject may be suffering from neuronal degeneration or neurotoxicity.

In one embodiment, the subject is suffering from amyloidoses. In another embodiment, the subject is suffering from Alzheimer's disease. In another embodiment, the subject is suffering from systemic amyloidosis. In a another embodiment, the subject is suffering from prion disease. In another embodiment, the subject is suffering from kidney failure. In another embodiment, the subject is suffering from diabetes. In a further embodiment, the subject is suffering from systemic lupus erythematosus or inflammatory lupus nephritis. In another embodiment, the subject is an obese subject (for example, is beyond the height/weight chart recommendations of the American Medical Association). In another embodiment, the subject is an aged subject (for example, a human over the age of 50, or preferably over the age 60). In a further embodiment, the subject is suffering from inflammation. In one embodiment, the subject is suffering from an AGE-related disease. In another embodiment, such AGE-related disease is manifest in the brain, retina, kidney, vasculature, heart, or lung. In another embodiment, the subject is suffering from Alzheimer's disease or a disease which is manifested by AGEs accumulating in the subject. In another embodiment, the subject is suffering from symptoms of diabetes such as soft tissue injury, reduced ability to see, cardiovascular disease, kidney disease, etc. Such symptoms would be known to one of skill in the art.

The administration of the compound may comprise intralesional, intraperitoneal, intramuscular or intravenous injection; infusion; liposome-mediated delivery; topical, intrathecal, gingival pocket, per rectum, intrabronchial, nasal, oral, ocular or otic delivery. In a further embodiment, the administration includes intrabronchial administration, anal, intrathecal administration or transdermal delivery. In another embodiment, the compound is administered hourly, daily, weekly, monthly or annually. In another embodiment, the effective amount of the compound comprises from about 0.000001 mg/kg body weight to about 100 mg/kg body weight.

The administration may be constant for a certain period of time or periodic and at specific intervals. The compound may be delivered hourly, daily, weekly, monthly, yearly (e.g. in a time release form) or as a one time delivery. The delivery may be continuous delivery for a period of time, e.g. intravenous delivery.

The carrier may be a diluent, an aerosol, a topical carrier, an aqeuous solution, a nonaqueous solution or a solid carrier.

The effective amount of the compound may comprise from about 0.000001 mg/kg body weight to about 100 mg/kg body weight. In one embodiment, the effective amount may comprise from about 0.001 mg/kg body weight to about 50 mg/kg body weight. In another embodiment, the effective amount may range from about 0.01 mg/kg body weight to about 10 mg/kg body weight. The actual effective amount will be based upon the size of the compound, the biodegradability of the compound, the bioactivity of the compound and the bioavailability of the compound. If the compound does not degrade quickly, is bioavailable and highly active, a smaller amount will be required to be effective. The effective amount will be known to one of skill in the art; it will also be dependent upon the form of the compound, the size of the compound and the bioactivity of the compound. One of skill in the art could routinely perform empirical activity tests for a compound to determine the bioactivity in bioassays and thus determine the effective amount.

The agent of the present invention may be delivered locally via a capsule which allows sustained release of the agent or the peptide over a period of time. Controlled or sustained release compositions include formulation in lipophilic depots (e.g., fatty acids, waxes, oils). Also comprehended by the invention are particulate compositions coated with polymers (e.g., poloxamers or poloxamines) and the agent coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

This invention provides a method of preventing and/or treating a disease involving β-sheet fibril formation in a subject which comprises administering to the subject a binding inhibiting amount of a compound capable of inhibiting binding of the β-sheet fibril to RAGE so as to thereby prevent and/or treat a disease involving β-sheet fibril formation in the subject. In one embodiment of this method, the disease involves β-sheet fibril formation other than Alzheimer's Disease. Accordingly, this invention also provides a method of preventing and/or treating a disease involving β-sheet fibril formation other than Alzheimer's Disease in a subject which comprises administering to the subject a binding inhibiting amount of a compound capable of inhibiting binding of the β-sheet fibril to RAGE so as to thereby prevent and/or treat a disease involving β-sheet fibril formation other than Alzheimer's Disease in the subject. In one embodiment, the compound is sRAGE or a fragment thereof. In another embodiment, the compound is an anti-RAGE antibody or portion thereof.

The present invention also provides for a method of treating or ameliorating symptoms in a subject which are associated with a disease, wherein the disease is atherosclerosis, hypertension, impaired wound healing, periodontal disease, male impotence, retinopathy and diabetes and complications of diabetes, which comprises administering to the subject an amount of the compound of the present invention or an agent capable of inhibiting the binding of a β-sheet fibril to RAGE effective to inhibit the binding so as to treat or ameliorate the disease or condition in the subject. The method may also prevent such conditions from occurring in the subject.

The diseases which may be treated or prevented with the methods of the present invention include but are not limited to diabetes, Alzheimer's Disease, senility, renal failure, hyperlipidemic atherosclerosis, neuronal cytotoxicity, Down's syndrome, dementia associated with head trauma, amyotrophic lateral sclerosis, multiple sclerosis, amyloidosis, an autoimmune disease, inflammation, a tumor, cancer, male impotence, wound healing, periodontal disease, neuopathy, retinopathy, nephropathy or neuronal degeneration. The condition may be associated with degeneration of a neuronal cell in the subject. The condition may be associated with formation of a β-sheet fibril or an amyloid fibril. The condition may be associated with aggregation of a β-sheet fibril or an amyloid fibril. The condition may be associated with diabetes. The condition may be diabetes, renal failure, hyperlipidemic atherosclerosis, associated with diabetes, neuronal cytotoxicity, Down's syndrome, dementia associated with head trauma, amyotrophic lateral sclerosis, multiple sclerosis, amyloidosis, an autoimmune disease, inflammation, a tumor, cancer, male impotence, wound healing, periodontal disease, neuopathy, retinopathy, nephropathy or neuronal degeneration. The advanced glycation endproduct (AGE) may be a pentosidine, a carboxymethyllysine, a carboxyethyllysine, a pyrallines, an imidizalone, a methylglyoxal, an ethylglyoxal.

The present invention also provides for a method for inhibiting periodontal disease in a subject which comprises administering topically to the subject a pharmaceutical composition which comprises sRAGE in an amount effective to accelerate wound healing and thereby inhibit periodontal disease. The pharmaceutical composition may comprise sRAGE in a toothpaste.

The present invention also encompasses a pharmaceutical composition which comprises a therapeutically effective amount of the compound linked to an antibody or portion thereof. In one embodiment, the antibody may be capable of specifically binding to RAGE. The antibody may be a monoclonal antibody, a polyclonal antibody. The portion or fragment of the antibody may comprise a F_{ab} fragment or a F_{c} fragment. The portion or fragment of the antibody may comprise a complementarity determining region or a variable region.

This invention provides a method of determining whether a compound inhibits binding of a β-sheet fibril to RAGE on the surface of a cell which comprises:
(a) immobilizing the β-sheet fibril on a solid matrix;
(b) contacting the immobilized β-sheet fibril with the compound being tested and a predetermined amount of RAGE under conditions permitting binding of β-sheet fibril to RAGE in the absence of the compound;
(c) removing any unbound compound and any unbound RAGE;
(d) measuring the amount of RAGE which is bound to immobilized β-sheet fibril;
(e) comparing the amount measured in step (d) with the amount measured in the absence of the compound, a decrease in the amount of RAGE bound to β-sheet fibril in the presence of the compound indicating that the compound inhibits binding of β-sheet fibril to RAGE.

The assay may be carried out wherein one of the components is bound or affixed to a solid surface. In one embodiment the peptide is affixed to a solid surface. The solid surfaces useful in this embodiment would be known to one of skill in the art. For example, one embodiment of a solid surface is a bead, a column, a plastic dish, a plastic plate, a microscope slide, a nylon membrane, etc. The material of which the solid surface is comprised is synthetic in one example.

The assay may be carried out in vitro, wherein one or more of the components are attached or affixed to a solid surface, or wherein the components are admixed inside of a cell; or wherein the components are admixed inside of an organism (i.e. a transgenic mouse). For example, the peptide may be affixed to a solid surface. The RAGE or the fragment thereof is affixed to a solid surface in another embodiment.

This invention provides a compound not previously known to inhibit binding of β-sheet fibril to RAGE determined to do so by the above method.

This invention provides a method of preparing a composition which comprises determining whether a compound inhibits binding of β-sheet fibril to RAGE by the above method and admixing the compound with a carrier.

This invention also provides for pharmaceutical compositions including therapeutically effective amounts of polypeptide compositions and compounds, together with suitable diluents, preservatives, solubilizers, emulsifiers, adjuvants and/or carriers. Such compositions may be liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the compound or composition. The choice of compositions will depend on the physical and chemical properties of the compound.

In the practice of any of the methods of the invention or preparation of any of the pharmaceutical compositions a “therapeutically effective amount” is an amount which is capable of preventing interaction of β-sheet fibril to RAGE in a subject. Accordingly, the effective amount will vary with the subject being treated, as well as the condition to be treated.

Controlled or sustained release compositions include formulation in lipophilic depots (e.g., fatty acids, waxes, oils). Also comprehended by the invention are particulate compositions coated with polymers (e.g., poloxamers or poloxamines) and the compound coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

When administered, compounds are often cleared rapidly from the circulation and may therefore elicit relatively short-lived pharmacological activity. Consequently, frequent injections of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently or in lower doses than with the unmodified compound.

Attachment of polyethylene glycol (PEG) to compounds is particularly useful because PEG has very low toxicity in mammals (Carpenter et al., 1971). For example, a PEG adduct of adenosine deaminase was approved in the United States for use in humans for the treatment of severe combined immunodeficiency syndrome. A second advantage afforded by the conjugation of PEG is that of effectively reducing the immunogenicity and antigenicity of heterologous compounds. For example, a PEG adduct of a human protein might be useful for the treatment of disease in other mammalian species without the risk of triggering a severe immune response. The polypeptide or composition of the present invention may be delivered in a microencapsulation device so as to reduce or prevent an host immune response against the polypeptide or against cells which may produce the polypeptide. The polypeptide or composition of the present invention may also be delivered microencapsulated in a membrane, such as a liposome.

Polymers such as PEG may be conveniently attached to one or more reactive amino acid residues in a protein such as the alpha-amino group of the amino terminal amino acid, the epsilon amino groups of lysine side chains, the sulfhydryl groups of cysteine side chains, the carboxyl groups of aspartyl and glutamyl side chains, the alpha-carboxyl group of the carboxy-terminal amino acid, tyrosine side chains, or to activated derivatives of glycosyl chains attached to certain asparagine, serine or threonine residues.

Numerous activated forms of PEG suitable for direct reaction with proteins have been described. Useful PEG reagents for reaction with protein amino groups include active esters of carboxylic acid or carbonate derivatives, particularly those in which the leaving groups are N-hydroxysuccinimide, p-nitrophenol, imidazole or 1-hydroxy-2-nitrobenzene-4-sulfonate. PEG derivatives containing maleimido or haloacetyl groups are useful reagents for the modification of protein free sulfhydryl groups. Likewise, PEG reagents containing amino hydrazine or hydrazide groups are useful for reaction with aldehydes generated by periodate oxidation of carbohydrate groups in proteins.

In one embodiment, the pharmaceutical carrier may be a liquid and the pharmaceutical composition would be in the form of a solution. In another equally preferred embodiment, the pharmaceutically acceptable carrier is a solid and the composition is in the form of a powder or tablet. In a further embodiment, the pharmaceutical carrier is a gel and the composition is in the form of a suppository or cream. In a further embodiment the active ingredient may be formulated as a part of a pharmaceutically acceptable transdermal patch.

A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are useful in sterile liquid form compositions for parenteral administration. The liquid carrier for pressurized compositions can be halogenated hydrocarbon or other pharmaceutically acceptable propellent.

Liquid pharmaceutical compositions which are sterile solutions or suspensions can be utilized by for example, intramuscular, intrathecal, epidural, intraperitoneal or subcutaneous injection. Sterile solutions can also be administered intravenously. The active ingredient may be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes, and coatings.

The active ingredient of the present invention (i.e., the compound identified by the screening method or composition thereof) can be administered orally in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like.

The active ingredient can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

When administered orally or topically, such agents and pharmaceutical compositions would be delivered using different carriers. Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients. The specific carrier would need to be selected based upon the desired method of deliver, e.g., PBS could be used for intravenous or systemic delivery and vegetable fats, creams, salves, ointments or gels may be used for topical delivery.

This invention also provides for pharmaceutical compositions including therapeutically effective amounts of protein compositions and/or agents capable of inhibiting the binding of an amyloid-β peptide with RAGE in the subject of the invention together with suitable diluents, preservatives, solubilizers, emulsifiers, adjuvants and/or carriers useful in treatment of neuronal degradation due to aging, a learning disability, or a neurological disorder. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the agent, complexation with metal ions, or incorporation of the agent into or onto particulate preparations of polymeric agents such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the agent or composition. The choice of compositions will depend on the physical and chemical properties of the agent capable of alleviating the symptoms in the subject.

The agent of the present invention may be delivered locally via a capsule which allows sustained release of the agent or the peptide over a period of time. Controlled or sustained release compositions include formulation in lipophilic depots (e.g., fatty acids, waxes, oils). Also comprehended by the invention are particulate compositions coated with polymers (e.g., poloxamers or poloxamines) and the agent coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

In one embodiment, the carrier comprises a diluent. In another embodiment, the carrier comprises, a virus, a liposome, a microencapsule, a polymer encapsulated cell or a retroviral vector. In another embodiment, the carrier is an aerosol, intravenous, oral or topical carrier, or aqueous or nonaqueous solution. For example, the compound is administered from a time release implant.

As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid®.

Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

This invention provides a method of determining whether a compound inhibits binding of β-sheet fibril to RAGE on the surface of a cell which comprises:
(a) contacting RAGE-transfected cells with the compound being tested under conditions permitting binding of the compound to RAGE;
(b) removing any unbound compound;
(c) contacting the cells with β-sheet fibril under conditions permitting binding of β-sheet fibril to RAGE in the absence of the compound;
(d) removing any unbound β-sheet fibril;
(e) measuring the amount of β-sheet fibril bound to the cells;
(f) separately repeating steps (c) through (e) in the absence of any compound being tested; (g) comparing the amount of β-sheet fibril bound to the cells from step (e) with the amount from step (f), wherein reduced binding of β-sheet fibril in the presence of the compound indicates that the compound inhibits binding of β-sheet fibril to RAGE.

In one embodiment of the above method, the cells are PC12 cells.

This invention provides a compound not previously known to inhibit binding of β-sheet fibril to RAGE determined to do so by the above method.

This invention provides a method of preparing a composition which comprises determining whether a compound inhibits binding of β-sheet fibril to RAGE by the above method and admixing the compound with a carrier.

The compounds, agents, peptides, antibodies, and fragments thereof of the present invention may be detectably labeled.

The detectable label may be a fluorescent label, a biotin, a digoxigenin, a radioactive atom, a paramagnetic ion, and a chemiluminescent label. It may also be labeled by covalent means such as chemical, enzymatic or other appropriate means with a moiety such as an enzyme or radioisotope. Portions of the above mentioned compounds of the invention may be labeled by association with a detectable marker substance (e.g., radiolabeled with ¹²⁵I or biotinylated) to provide reagents useful in detection and quantification of compound or its receptor bearing cells or its derivatives in solid tissue and fluid samples such as blood, cerebral spinal fluid or urine.

The present invention also provides for a transgenic nonhuman mammal whose germ or somatic cells contain a nucleic acid molecule which encodes an RAGE peptide or a biologically active variant thereof, introduced into the mammal, or an ancestor thereof, at an embryonic stage. In one embodiment, the nucleic acid molecule which encodes RAGE polypeptide is overexpressed in the cells of the mammal. In another embodiment, the nucleic acid molecule encodes human RAGE peptide. In another embodiment, the active variant comprises a homolog of RAGE.

The present invention also provides for a transgenic nonhuman mammal whose germ or somatic cells have been transfected with a suitable vector with an appropriate sequence designed to reduce expression levels of RAGE peptide below the expression levels of that of a native mammal. In one embodiment, the suitable vector contains an appropriate piece of cloned genomic nucleic acid sequence to allow for homologous recombination. In another embodiment, the suitable vector encodes a ribozyme capable of cleaving an RAGE mRNA molecule or an antisense molecule which comprises a sequence antisense to naturally occurring EN-RAGE mRNA sequence.

The compound of the present invention may be used to treat wound healing in subjects. The wound healing may be associated with various diseases or conditions. The diseases or conditions may impair normal wound healing or contribute to the existence of wounds which require healing. The subjects may be treated with the peptides or agents or pharmaceutical compositions of the present invention in order to treat slow healing, recalcitrant periodontal disease, wound healing impairment due to diabetes and wound healing impairments due to autoimmune disease. The present invention provides compounds and pharmaceutical compositions useful for treating impaired wound healing resultant from aging. The effect of topical administration of the agent can be enhanced by parenteral administration of the active ingredient in a pharmaceutically acceptable dosage form.

The pathologic hallmarks of Alzheimer's disease (AD) are intracellular and extracellular deposition of filamentous proteins which closely correlates with eventual neuronal dysfunction and clinical dementia (for reviews see Goedert, 1993; Haass et al., 1994; Kosik, 1994; Trojanowski et al., 1994; Wischik, 1989). Amyloid-β peptide (Aβ) is the principal component of extracellular deposits in AD, both in senile/diffuse plaques and in cerebral vasculature. Aβ has been shown to promote neurite outgrowth, generate reactive oxygen intermediates (ROIs), induce cellular oxidant stress, lead to neuronal cytotoxicity, and promote microglial activation (Behl et al., 1994; Davis et al., 1992; Hensley, et al., 1994; Koh, et al., 1990; Koo et al., 1993; Loo et al., 1993; Meda et al., 1995; Pike et al., 1993; Yankner et al., 1990). For Aβ to induce these multiple cellular effects, it is likely that plasma membranes present a binding protein(s) which engages Aβ. In this context, several cell-associated proteins, as well as sulfated proteoglycans, can interact with Aβ. These include: substance P receptor, the serpin-enzyme complex (SEC) receptor, apolipoprotein E, apolipoprotein J (clusterin), transthyretin, alpha-1 anti-chymotrypsin, β-amyloid precursor protein, and sulphonates/heparan sulfates (Abraham et al., 1988; Fraser et al., 1992; Fraser et al., 1993; Ghiso et al., 1993; Joslin et al., 1991; Kimura et al., 1993; Kisilevsky et al., 1995; Strittmatter et al., 1993a; Strittmatter et al., 1993b; Schwarzman et al., 1994; Snow et al., 1994; Yankner et al., 1990). Of these, the substance P receptor and SEC receptor might function as neuronal cell surface receptors for Aβ, though direct evidence for this is lacking (Fraser et al., 1993; Joslin et al., 1991; Kimura et al., 1993; Yankner et al., 1990). In fact, the role of substance P receptors is controversial, and it is not known whether Aβ alone interacts with the receptor, or if costimulators are required (Calligaro et al., 1993; Kimura et al., 1993; Mitsuhashi et al., 1991) and the SEC receptor has yet to be fully characterized.

In certain embodiments of the present invention, the subject may be suffering from clinical aspects as described hereinbelow and as further described in Harper's Biochemistry, R. K. Murray, et al. (Editors) 21st Edition, (1988) Appelton & Lange, East Norwalk, Conn. Such clinical aspects may predispose the subject to atherosclerosis or to accelerated atherosclerosis. Thus, such subjects would benefit from the administration of a polypeptide derived from sRAGE in an effective amount over an effective time.

The subject of the present invention may demonstrate clinical signs of atherosclerosis, hypercholesterolemia or other disorders as discussed hereinbelow.

Clinically, hypercholesterolemia may be treated by interrupting the enterohepatic circulation of bile acids. It is reported that significant reductions of plasma cholesterol can be effected by this procedure, which can be accomplished by the use of cholestyramine resin or surgically by the ileal exclusion operations. Both procedures cause a block in the reabsorption of bile acids. Then, because of release from feedback regulation normally exerted by bile acids, the conversion of cholesterol to bile acids is greatly enhanced in an effort to maintain the pool of bile acids. LDL (low density lipoprotein) receptors in the liver are up-regulated, causing increased uptake of LDL with consequent lowering of plasma cholesterol.

The peptides, agents and pharmaceutical compositions of the present invention may be used as therapeutic agents to inhibit symptoms of diseases in a subject associated with cholesterol metabolism, atherosclerosis or coronary heart disease. Some symptoms of such diseases which may be inhibited or ameliorated or prevented through the administration of the agents and pharmaceutical compositions of the present invention are discussed hereinbelow. For example, the agents and pharmaceutical compositions of the present invention may be administered to a subject suffering from symptoms of coronary heart disease in order to protect the integrity of the endothelial cells of the subject and thereby inhibit the symptoms of the coronary heart disease.

Many investigators have demonstrated a correlation between raised serum lipid levels and the incidence of coronary heart disease and atherosclerosis in humans. Of the serum lipids, cholesterol has been the one most often singled out as being chiefly concerned in the relationship. However, other parameters such as serum triacylglycerol concentration show similar correlations. Patients with arterial disease can have any one of the following abnormalities: (1) elevated concentrations of VLDL (very low density lipoproteins) with normal concentrations of LDL; (2) elevated LDL with Normal VLDL; (3) elevation of both lipoprotein fractions. There is also an inverse relationship between HDL (high density lipoproteins) (HDL₂) concentrations and coronary heart disease, and some consider that the most predictive relationship is the LDL:HDL cholesterol ratio. This relationship is explainable in terms of the proposed roles of LDL in transporting cholesterol to the tissues and of HDL acting as the scavenger of cholesterol.

Atherosclerosis is characterized by the deposition of cholesterol and cholesteryl ester of lipoproteins containing apo-B-100 in the connective tissue of the arterial walls. Diseases in which prolonged elevated levels of VLDL, IDL, or LDL occur in the blood (e.g., diabetes, mellitus, lipid nephrosis, hypothyroidism, and other conditions of hyperlipidemia) are often accompanied by premature or more sever atherosclerosis.

Experiments on the induction of atherosclerosis in animals indicate a wide species variation in susceptibility. The rabbit, pig, monkey, and humans are species in which atherosclerosis can be induced by feeding cholesterol. The rat, dog, mouse and cat are resistant. Thyroidectomy or treatment with thiouracil drugs will allow induction of atherosclerosis in the dog and rat. Low blood cholesterol is a characteristic of hyperthyroidism.

Hereditary factors play the greatest role in determining individual blood cholesterol concentrations, but of the dietary and environmental factors that lower blood cholesterol, the substitution in the diet of polyunsaturated fatty acids for some of the saturated fatty acids has been the most intensely studied.

Naturally occurring oils that contain a high proportion of linoleic acid are beneficial in lowering plasma cholesterol and include peanut, cottonseed, corn, and soybean oil whereas butterfat, beef fat, and coconut oil, containing a high proportion of saturated fatty acids, raise the level. Sucrose and fructose have a greater effect in raising blood lipids, particularly triacylglycerols, than do other carbohydrates.

The reason for the cholesterol-lowering effect of polyunsaturated fatty acids is still not clear. However, several hypotheses have been advanced to explain the effect, including the stimulation of cholesterol excretion into the intestine and the stimulation of the oxidation of cholesterol to bile acids. It is possible that cholesteryl esters of polyunsaturated fatty acids are more rapidly metabolized by the liver and other tissues, which might enhance their rate of turnover and excretion. There is other evidence that the effect if largely due to a shift in distribution of cholesterol from the plasma into the tissues because of increased catabolic rate of LDL. Saturated fatty acids cause the formation of smaller VLDL particles that contain relatively more cholesterol, and they are utilized by extrahepatic tissues at a slower rate than are larger particles. All of these tendencies may be regarded as atherogenic.

Additional factors considered to play a part in coronary heart disease include high blood pressure, smoking, obesity, lack of exercise, and drinking soft as opposed to hard water. Elevation of plasma free fatty acids will also lead to increase VLDL secretion by the liver, involving extra triacylglycerol and cholesterol output into the circulation. Factors leading to higher or fluctuating levels of free fatty acids include emotional stress, nicotine from cigarette smoking, coffee drinking, and partaking of a few large meals rather than more continuous feeding. Premenopausal women appear to be protected against many of these deleterious factors, possibly because they have higher concentrations of HDL than do men and postmenopausal women.

When dietary measures fail to achieve reduced serum lipid levels, the use of hypolipidemic drugs may be resorted to. Such drugs may be used in conjunction with the agents and pharmaceutical compositions of the present invention, i.e., such drugs may be administered to a subject along with the agents of the present invention. Several drugs are known to block the formation of cholesterol at various stages in the biosynthetic pathway. Many of these drugs have harmful effects, but the fungal inhibitors of HMG-CoA reductase, compactin and mevinolin, reduce LDL cholesterol levels with few adverse effects. Sitosterol is a hypocholesterolemic agent that acts by blocking the absorption of cholesterol in the gastrointestinal tract. Resins such as colestipol and cholestyramine (Questran) prevent the reabsorption of bile salts by combining with them, thereby increasing their fecal loss. Neomycin also inhibits reabsorption of bile salts. Clofibrate and gembivrozil exert at least part of their hypolipidemic effect by diverting the hepatic flow of free fatty acids from the pathways of esterification into those of oxidation, thus decreasing the secretion of triacylglycerol and cholesterol containing VLDL by the liver. In addition, they facilitate hydrolysis of VLDL triacylglycerols by lipoprotein lipase. Probucol appears to increase LDL catabolism via receptor-independent pathways. Nicotinic acid reduces the flux of FFA by inhibiting adipose tissue lipolysis, thereby inhibiting VLDL production by the liver.

A few individuals in the population exhibit inherited defects in their lipoproteins, leading to the primary condition of whether hypo- or hyperlipoproteinemia. Many others having defects such as diabetes mellitus, hypothyroidism, and atherosclerosis show abnormal lipoprotein patterns that are very similar to one or another of the primary inherited conditions. Virtually all of these primary conditions are due to a defect at one or another stage in the course of lipoprotein formation, transport, or destruction. Not all of the abnormalities are harmful.

### Hypolipoproteinemia:

1. Abetalipoproteinemia—This is a rare inherited disease characterized by absence of β-lipoprotein (LDL) in plasma. The blood lipids are present in low concentrations—especially acylglycerols, which are virtually absent, since no chylomicrons or VLDL are formed. Both the intestine and the liver accumulate acylglycerols. Abetalipoproteinemia is due to a defect in apoprotein B synthesis.

2. Familial hypobetalipoproteinemia—In hypobetalipoproteinemia, LDL concentration is between 10 and 50% of normal, but chylomicron formation occurs. It must be concluded that apo-B is essential for triacylglycerol transport. Most individuals are healthy and long-lived.

3. Familial alpha-lipoprotein deficiency (Tangier disease)—In the homozygous individual, there is near absence of plasma HDL and accumulation of cholesteryl esters in the tissues. There is no impairment of chylomicron formation or secretion of VLDL by the liver. However, on electrophoresis, there is no pre-β-lipoprotein, but a broad β-band is found containing the endogenous triacylglycerol. This is because the normal pre-β-band contains other apo-proteins normally provided by HDL. Patients tend to develop hypertriacylglycerolemia as a result of the absence of apo-C-II, which normally activates lipoprotein lipase.

### Hyperlipoproteinemia:

1. Familial lipoprotein lipase deficiency (type I)— This condition is characterized by very slow clearing of chylomicrons from the circulation, leading to abnormally raised levels of chylomicrons. VLDL may be raised, but there is a decrease in LDL and HDL. Thus, the condition is fat-induced. It may be corrected by reducing the quantity of fat and increasing the proportion of complex carbohydrate in the diet. A variation of this disease is caused by a deficiency in apo-C-II, required as a cofactor for lipoprotein lipase.

2. Familial hypercholesterolemia (type II)-Patients are characterized by hyperbetalipoproteinemia (LDL), which is associated with increased plasma total cholesterol. There may also be a tendency for the VLDL to be elevated in type IIb. Therefore, the patient may have somewhat elevated triacylglycerol levels but the plasma—as is not true in the other types of hyperlipoproteinemia—remains clear. Lipid deposition in the tissue (e.g., xanthomas, atheromas) is common. A type II pattern may also arise as a secondary result of hypothyroidism. The disease appears to be associated with reduced rates of clearance of LDL from the circulation due to defective LDL receptors and is associated with an increased incidence of atherosclerosis. Reduction of dietary cholesterol and saturated fats may be of use in treatment. A disease producing hypercholesterolemia but due to a different cause is Wolman's disease (cholesteryl ester storage disease). This is due to a deficiency of cholesteryl ester hydrolase in lysosomes of cells such as fibroblasts that normally metabolize LDL.

3. Familial type III hyperlipoproteinemia (broad beta disease, remnant removal disease, familial dysbetalipoproteinemia)—This condition is characterized by an increase in both chylomicron and VLDL remnant; these are lipoproteins of density less than 1.019 but appear as a broad β-band on electrophoresis (β-VLDL). They cause hypercholesterolemia and hypertriacylglycerolemia. Xanthomas and atherosclerosis of both peripheral and coronary arteries are present. Treatment by weight reduction and diets containing complex carbohydrates, unsaturated fats, and little cholesterol is recommended. The disease is due to a deficiency in remnant metabolism by the liver caused by an abnormality in apo-E, which is normally present in 3 isoforms, E2, E3, and E4. Patients with type III hyperlipoproteinemia possess only E2, which does not react with the E receptor.

4. Familial hypertriacylglycerolemia (type IV)—This condition is characterized by high levels of endogenously produced triacylglycerol (VLDL). Cholesterol levels rise in proportion to the hypertriacylglycerolemia, and glucose intolerance is frequently present. Both LDL and HDL are subnormal in quantity. This lipoprotein pattern is also commonly associated with coronary heart disease, type II non-insulin-dependent diabetes mellitus, obesity, and many other conditions, including alcoholism and the taking of progestational hormones. Treatment of primary type IV hyperlipoproteinemia is by weight reduction; replacement of soluble diet carbohydrate with complex carbohydrate, unsaturated fat, low-cholesterol diets; and also hypolipidemic agents.

5. Familial type V hyperlipoproteinemia—The lipoprotein pattern is complex, since both chylomicrons and VLDL are elevated, causing both triacylglycerolemia and cholesterolemia. Concentrations of LDL and HDL are low. Xanthomas are frequently present, but the incidence of atherosclerosis is apparently not striking. Glucose tolerance is abnormal and frequently associated with obesity and diabetes. The reason for the condition, which is familial, is not clear. Treatment has consisted of weight reduction followed by a diet not too high in either carbohydrate or fat.

It has been suggested that a further cause of hypolipoproteinemia is overproduction of apo-B, which can influence plasma concentrations of VLDL and LDL.

6. Familial hyperalphalipoproteinemia—This is a rare condition associated with increased concentrations of HDL apparently beneficial to health.

Familial Lecithin Cholesterol Acyltransferase (LCAT) Deficiency: In affected subjects, the plasma concentration of cholesteryl esters and lysolecithin is low, whereas the concentration of cholesterol and lecithin is raised. The plasma tends to be turbid. Abnormalities are also found in the lipoproteins. One HDL fraction contains disk-shaped structures in stacks or rouleaux that are clearly nascent HDL unable to take up cholesterol owing to the absence of LCAT. Also present as an abnormal LDL subfraction is lipoprotein-X, otherwise found only in patients with cholestasis. VLDL are also abnormal, migrating as β-lipoproteins upon electrophoresis (β-VLDL). Patients with parenchymal liver disease also show a decrease of LCAT activity and abnormalities in the serum lipids and lipoproteins.

### Atherosclerosis:

In one embodiment of the present invention, the subject may be predisposed to atherosclerosis. This predisposition may include genetic predisposition, environmental predisposition, metabolic predisposition or physical predisposition. There have been recent reviews of atherosclerosis and cardiovascular disease. For example: Keating and Sanguinetti, (May 1996) Molecular Genetic Insights into Cardiovascular Disease, Science 272:681-685 is incorporated by reference in its entirety into the present application. The authors review the application of molecular tools to inherited forms of cardiovascular disease such as arrhythmias, cardiomyopathies, and vascular disease. Table 1 of this reference includes cardiac diseases and the aberrant protein associated with each disease. The diseases listed are: LQT disease, familial hypertrophic cardiomyopathy; duchenne and Becker muscular dystrophy; Barth syndrome Acyl-CoA dehydrogenase deficiencies; mitochondrial disorders; familial hypercholesterolemia; hypobetalipoproteinemia; homocystinuria; Type III hyperlipoproteinemia; supravalvular aortic stenosis; Ehler-Danlos syndrome IV; Marfa syndrome; Heredity hemorrhagic telangiectasia. These conditions are included as possible predispositions of a subject for atherosclerosis.

Furthermore, mouse models of atherosclerosis are reviewed in Breslow (1996) Mouse Models of Atherosclerosis, Science 272:685. This reference is also incorporated by reference in its entirety into the present application. Breslow also includes a table (Table 1) which recites various mouse models and the atherogenic stimulus. For example, mouse models include C57BL/6; Apo E deficiency; ApoE lesion; ApoE R142C; LDL receptor deficiency; and HuBTg. One embodiment of the present invention is wherein a subject has a predisposition to atherosclerosis as shown by the mouse models presented in Breslow's publication.

Gibbons and Dzau review vascular disease in Molecular Therapies for Vascular Disease, Science Vol. 272, pages 689-693. In one embodiment of the present invention, the subject may manifest the pathological events as described in Table 1 of the Gibbons and Dzau publication. For example, the subject may have endothelial dysfunction, endothelial injury, cell activation and phenotypic modulation, dysregulated cell growth, dysregulated apoptosis, thrombosis, plaque rupture, abnormal cell migration or extracellular or intracellular matrix modification.

In another embodiment of the present invention, the subject may have diabetes. The subject may demonstrate complications associated with diabetes. Some examples of such complications include activation of endothelial and macrophage AGE receptors, altered lipoproteins, matrix, and basement membrane proteins; altered contractility and hormone responsiveness of vascular smooth muscle; altered endothelial cell permeability; sorbitol accumulation; neural myoinositol depletion or altered Na-K ATPase activity. Such complications are discussed in a recent publication by Porte and Schwartz, Diabetes Complications: Why is Glucose potentially Toxic?, Science, Vol. 272, pages 699-700.

This invention is illustrated in the Experimental Details section which follows. These sections are set forth to aid in an understanding of the invention but are not intended to, and should not be construed to, limit in any way the invention as set forth in the claims which follow thereafter. One skilled in the art will readily appreciate that the specific methods and results discussed are merely illustrative of the invention as described more fully in the claims which follow thereafter.

### Experimental Details

Fibrils composed of amyloid β-peptide, serum amyloid A, amylin and prion protein share β-sheet structure and are characteristic of the extracellular pathology of amyloidoses, such as Alzheimer's disease, systemic amyloidosis, and prion disease. Abundant accumulations of fibrils observed late in the course of these disorders are likely to nonspecifically destabilize cell membranes. We hypothesized that early in the course of amyloidoses, interaction of fibrils with cellular surfaces might be orchestrated by specific binding sites/receptors. RAGE, a multiligand immunoglobulin superfamily receptor, is shown to bind fibrils composed of a range of amyloidogenic peptides following their assembly into β-sheet-containing structures. Fibril-RAGE interaction at the cell surface triggers receptor-dependent signal transduction mechanisms and increased vulnerability to cytotoxicity. In a model of systemic amyloidosis, blockade of fibril-RAGE interaction in vivo suppressed cellular stress and amyloid A fibril accumulation. These data suggest that cell surface RAGE is a focal point for interaction with fibrils, rendering amyloid pathogenic by a receptor-dependent mechanism.

### Methods

### RAGE-Related Reagents

PC12 cells (ATCC; a clone which did not express RAGE) were stably transfected with pcDNA3 alone or pcDNA3/wt (human) RAGE (Schmidt et al., 1999) according to the manufacturer's instructions (GIBCO/BRL), and clones were selected with high levels of RAGE expression. Transient transfection experiments with neuroblastoma cells utilized pcDNA3/wtRAGE or a construct encoding TD-RAGE. TD-RAGE was made with a TA cloning kit from InVitrogen using 5′ and 3′-primers for the RAGE cDNA, cleaved with Kpn1-Xhol, and inserted into the pcDNA3 vector. Murine and human sRAGE were expressed using the baculovirus system and purified to homogeneity (Hori et al., 1995; Park et al., 1998). To prepare isolated RAGE domains, human RAGE cDNA encoding the V-, C- or C′-domain was inserted into the EcoR1 site of pGEX4T vector containing GST. Fusion proteins, V-GST, C-GST and C′-GST, were expressed in E. Coli, purified on a glutathione-Sepharose column, and cleaved with thrombin (Pharmacia). RAGE domains were then purified to homogeneity using glutathione-Sepharose, and characterized by SDS-PAGE and N-terminal sequencing. The numbering system for amino acids in RAGE assigns #1 to the initial methionine residue. Monospecific polyclonal rabbit anti-human and anti-mouse RAGE IgG, against human or murine sRAGE, were prepared as described (Hori et al., 1995).

### Immunoblotting, Immunocytochemistry, and Electron Microscopy

Immunoblotting utilized nonfat dry milk and either rabbit anti-human RAGE IgG (3.3 μg/ml), anti-phosphorylated ERK M (5 μg/ml; Upstate Biotechnology) or anti-apoSAA IgG (1 μg/ml; this antibody crossreacts with amyloid A fibrils isolated from murine splenic tissue, and recognizes both apoSAA1 and apoSAA2) (Blacker et al., 1998). Sites of primary antibody binding were identified with peroxidase-conjugated anti-rabbit IgG (1:2000 dilution; Sigma) by the ECL method (Amersham), and autoradiograms were analyzed by laser densitometry. Immunohistological analysis of paraformaldehyde-fixed, paraffin-embedded sections (5-6 μm) employed rabbit anti-mouse IL-6 IgG (50 μg/ml; generously provided by Dr. Gerald Fuller, Univ. of Alabama, Birmingham Ala.), goat anti-mouse M-CSF IgG (4 μg/ml; Santa Cruz), rabbit anti-apoSAA IgG (1 ug/ml) and anti-RAGE IgG (50 μg/ml), and the Biotin-ExtrAvidin Alkaline Phosphatase Kit (Sigma). Quantitation of microscopic images was accomplished with the Universal Imaging System.

For electron microscopic analysis, PC12/RAGE or PC12/vector cells briefly fixed (2 min) in paraformaldehyde (2%) were incubated with preformed Aβ(1-40) fibrils for 4 hrs, washed, removed from the dish by scraping, pelleted by centrifugation, and then embedded in EPON resin. Sections were cut (15-17 nm), negatively stained with phosphotungstic acid (1%), and visualized in a JE100CX electron microscope. In certain experiments, after incubation of cells with Aβ fibrils, rabbit anti-RAGE IgG (30 μg/ml) was added for 1 hr at 37° C., and then goat anti-rabbit IgG conjugated to colloidal gold (10 nm; 1:100) was added for another 30 min at 37° C. Sections were then fixed and stained as above.

### Preparation of Fibrils and Thioflavine T Binding

Aβ(1-40) fibrils were made by dissolving Aβ(1-40) (2.2 mg/ml) in distilled water, neutralizing the pH to 7.4 with phosphate buffer, and incubating for 4 days at 37° C. Fibril formation was assessed by electron microscopy and secondary structure was determined by CD spectroscopy. Fibril preparations were pelletted by centrifugation, resuspended in phosphate-buffered saline (PBS; pH 7.4), subjected to five strokes of the sonicator, aliquoted and frozen at −20° C. Following thawing, preparations were used immediately for experiments. Prion peptide (residues 109-141) (Biosynthesis, Inc.), serum amyloid A peptide (residues 2-15)(Biosynthesis, Inc.) and human amylin (MRL, Inc.) fibrils were made similarly, except the peptides were initially dissolved in trifluoroacetic acid (0.1%):acetone (1:1), lyophilized and then resuspended in PBS at 2.0 mg/ml (amylin and amyloid A peptide) and 2.5 mg/ml (prion peptide). The concentration of fibrillar preparations indicated in the text/figures is derived from that of the monomer initially added to the mixture to make fibrils.

Mouse apoSAA1, apoSAA2, apoSAAce/j (Sipe et al., 1993), apoA-I and apoA-II were prepared from HDL isolated from plasma of C57BL/6 and CE/J mice subjected to acute phase stimulation by intraperitoneal injection of lipopolysaccharide (E. Coli 0111:B4, Difco Laboratories). HDL was isolated from plasma by KBr density centrifugation (Strachen et al., 1988; deBeer et al., 1993), and delipidated HDL was separated on a Sephacryl S200 column equilibrated with urea (8 M)/Tris-HCl (10 mM; pH 8.2). Peak apoSAA samples were fractionated on DEAE-Sephacel in the same buffer, and eluted with a linear gradient of NaCl to 150 mM. Fractions were analyzed by SDS-PAGE/immunoblotting and isoelectic focussing to verify SAA isoform. Amyloid A fibrils were purified from spleens of mice treated with AEF/SN as described (Prelli et al., 1987).

Fluorometric quantitation of Aβ fibrillogenesis utilized the thioflavine T binding assay, in which binding causes a shift in the emission spectrum and fluorescent signal proportional to the mass of amyloid formed (LeVine, 1993; Soto and Castano, 1996). Aliquots of Aβ (1.0 μg/μl) were incubated at room temperature in PBS with the indicated concentrations of sRAGE, soluble polio virus receptor (Gomez et al., 1993), or nonimmune rabbit F(ab′)₂. After incubation, samples were added to 50 mM glycine (pH 9.0) containing thioflavine T in a final volume of 2 ml. Immediately thereafter, fluorescence was monitored with excitation at 435 nm and emission at 485 nm in a Perkin Elmer model LS50B fluorescence spectrometer. A time scan of fluorescence was performed and three values after the decay reached a plateau (280, 290 and 300 secs) were averaged following subtraction of the background fluorescence of 2 μM thioflavine T. Albumin was without effect on thioflavine T fluorescence in the presence of Aβ when used in place of sRAGE at the same molar concentrations.

### RAGE-Fibril Binding Assays

Binding of β-sheet fibrils to PC12/RAGE or PC12/vector cells was studied by incubating cultures with preformed Aβ(1-40)-, prion peptide-, amylin- or serum amyloid A-derived fibrils in PBS for 4 hrs at 37° C., removing unbound fibrils by washing, and then addition of Congo red (25 μM) for 30 min at room temperature. Optical density was then measured with 490 nm/540 nm, and Congo red binding to cell-associated fibrils was determined as described (Wood et al., 1995). Binding assays were also performed in a purified system by incubating protein preparations in carbonate/bicarbonate buffer in microtiter wells (Nunc Maxisorp) for 20 hrs at 4° C. to allow adsorption, blocking with PBS containing albumin (10 mg/ml) for 2 hrs at 37° C., and then adding sRAGE in Minimal Essential Medium with HEPES (10 mM; pH 7.4) and fatty acid-free bovine serum albumin (1 mg/ml) for 2 hrs at 37° C. The reaction mixture was removed, wells were washed with ice-cold PBS containing Tween-20 (0.05%) four times over 30 sec. Bound sRAGE was eluted with Nonidet-P40 (1%) for 5 min at 37° C., and RAGE antigen was quantitated by ELISA or, when ¹²⁵I-sRAGE was employed, by counting radioactivity. Radiolabelling of sRAGE was accomplished by the Iodobead method (Pierce) (Yan et al., 1996). In other experiments, recombinant RAGE V-domain was similarly radiolabelled and employed in binding studies. Another binding assay exploited the fluorescent quenching of RAGE following its interaction with ligands. Intrinsic RAGE fluorescence (0.5_M) in 0.3 ml of Tris (5 mM, pH 7.4) at room temperature was studied at excitation 290 nm and emission over 300-420 nm, with a maximum at 355 nm. Binding experiments were done by adding lyophilized aliquots of peptide to sRAGE, and recording the fluorescence change. Binding parameters were plotted by determining the fluorescence change at 355 nm versus the concentration of added peptide, and data was analyzed (Klotz and Hunston, 1984) using nonlinear least squares analysis and a one-site model.

### EMSA, NF-κB-Driven Gene Expression and DNA Fragmentation Assays

EMSA was performed using nuclear extracts from cultured cells or splenic tissue and a ³²P-labelled consensus probe for NF-kB as described (Yan et al., 1996). To assess the effect of β-sheet fibril-RAGE interaction on gene expression, transient transfection experiments were performed with a construct under control of four NF-kB consensus sites linked to luciferase (InVitrogen). Transfection was performed with lipofectamine (GIBCO/BRL), cultures were then incubated for 48 hrs at 37° C., preformed fibrils were added, the incubation period was continued for 6 hrs longer, and chemiluminescence was determined with a luminometer. Other transient transfection studies were performed similarly. DNA fragmentation was determined using the Cell Death ELISA for cytoplasmic histone-associated DNA fragments (Boehringer Mannheim) and by the TUNEL method (Yan et al., 1997).

Murine model of systemic amyloidosis C57BL6/J mice (2-4 months) were injected with AEF (100 μg)/SN (0.5 ml of 2% solution) for 5 days to induce amyloid deposition, and were sacrificed at day 5 (Kisilevsky et al., 1995; Kindy et al., 1995; Kindy and Rader, 1998). Mice were treated with recombinant murine sRAGE, prepared as described above, saline or mouse serum albumin injected intraperitoneally once daily starting at day −1 (day 0 indicates the start of AEF/SN) and continuing up to day 4. For analysis of amyloid deposition, mice were perfused with ice-cold saline followed by buffered paraformaldehyde (4%), and spleens were post-fixed for 24 hrs in paraformaldehyde (4%) (Kindy and Rader, 1998). Tissues were embedded in paraffin and processed as above. Congo red staining was performed as described (Kindy et al., 1995), and quantitation of amyloid burden utilized image analysis carried out on immunostained (anti-apoSAA IgG) and Congo red-stained (polarized light) sections (Kisilevsky et al., 1995; Kindy and Rader, 1998). Amyloid burden in tissue sections was compared with standards for quantitation. For Northern analysis, the spleen was cut into small pieces, immersed in Trizol (Gibco BRL), homogenized, and total RNA was extracted and subjected to electrophoresis (0.8% agarose). RNA was transferred to Duralon-UV membranes (Stratagene), and membranes were then hybridized with ³²P-labelled cDNA probes for murine RAGE, HO-1, IL-6, and M-CSF.

### Results

RAGE Interaction with Aβ Fibrils

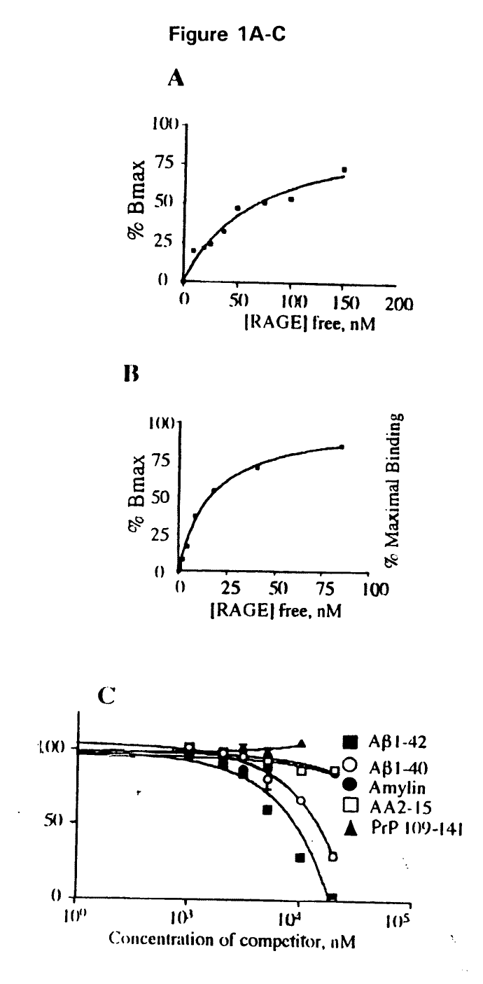

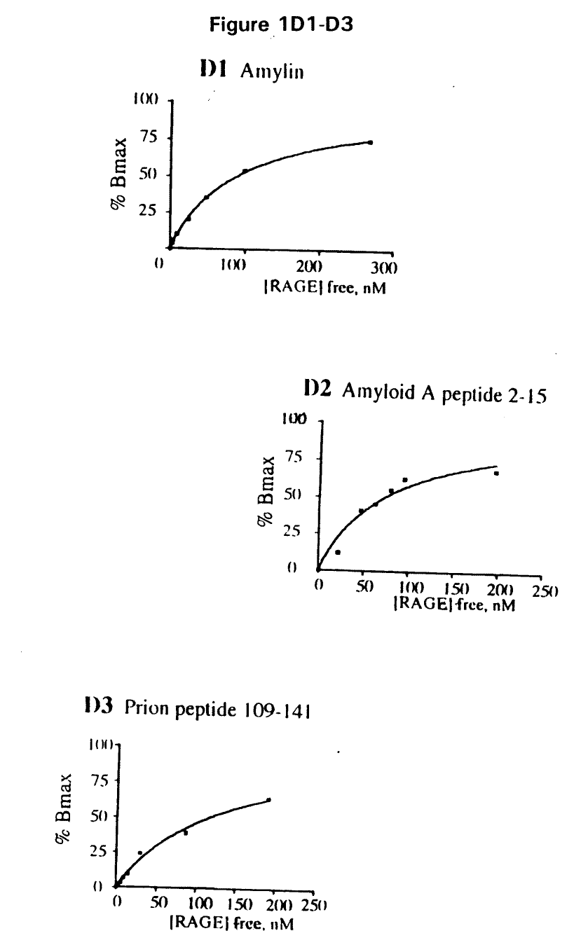

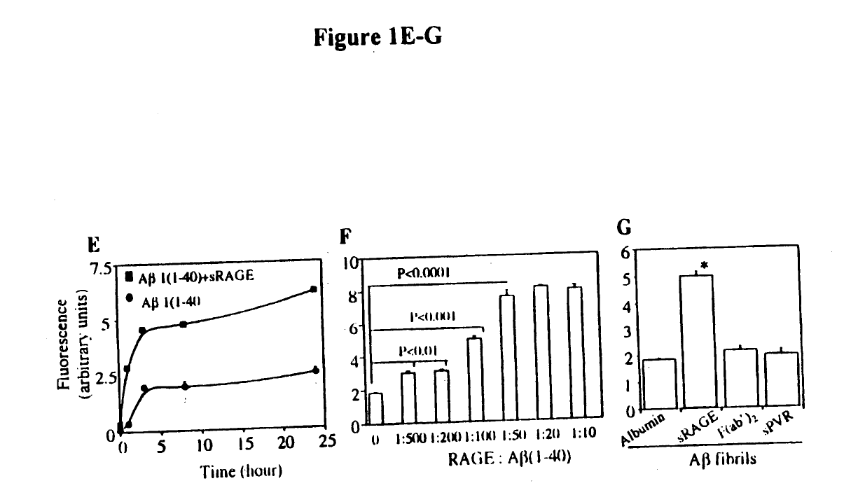

In a previous study, it was demonstrated that RAGE bound Aβ with high affinity (Yan et al., 1996). Because of the close association of fibrillar Aβ, as well as other amyloids, with cellular stress and cytotoxicity (Pike et al., 1993; Yankner, 1996), we sought to determine whether RAGE bound such fibrils. The nature of fibrillar material renders analysis of binding parameters only approximate, though the presence of dose-dependent, saturable binding versus nonspecific binding can be ascertained. For this reason, several different assays were developed to analyze the interaction of Aβ with RAGE in a purified system, including direct measurement of ¹²⁵I-labelled sRAGE binding to immobilized Aβ, an ELISA to quantitate nonlabelled sRAGE bound to Aβ, and a fluorometric assay based on quenching of intrinsic RAGE fluorescence consequent to the interaction with Aβ. Soluble RAGE bound to both freshly dissolved nonaggregated Aβ (1-40) and to preformed Aβ (1-40) fibrils with apparent K_{d}'s of −66-68 and =18 nM, respectively (FIG. 1A-B by the ELISA method, and Table 1, by the fluorescence method). Similar binding parameters were obtained using the three binding assays mentioned above. A peptide containing the reverse sequence of Aβ (1-40), designated Aβ (40-1), did not bind RAGE (Table 1), nor did several other control peptides of hydrophobicity similar to Aβ (not shown).

To analyze the specificity of binding between Aβ and sRAGE, other peptides also known for their ability to form amyloid fibrils were studied. Human amylin and fragments of the prion protein (a peptide spanning residues 109-141) and serum amyloid A (a peptide spanning residues 2-15) were aggregated in vitro forming β-sheet, amyloid-like fibrils based on circular dichroism and electron microscopic analysis (not shown)(Sipe, 1992; Ghiso et al., 1994; Soto et al., 1995; Prusiner, 1998). None of these freshly solubilized peptides was able to bind sRAGE (Table 1) or to displace the interaction of Aβ with sRAGE (FIG. 1C).

However, when the peptides were preincubated under conditions promoting fibril formation, sRAGE bound to each of the fibrils with similar affinity to that observed for Aβ fibrils; K_{d}'s=68 and 69, and 127 nM for fibrils of amylin, amyloid A and prion peptide (FIGS. 1D1-3). Since the peptides do not display sequence homology, these results suggest that the receptor recognition unit is a structural motif common to amyloid fibrils. It is widely accepted that amyloid fibrils are assembled by interactions between the β-strands of several peptide monomers forming aggregated intermolecular β-sheets, a structure known as cross-β conformation (Kirschner et al., 1986; Serpell et al., 1997). To determine whether any protein adopting a β-sheet structure would interact with sRAGE, binding studies were performed with erabutoxin B, a well-known all-β-sheet protein that does not form amyloid (Inagaki et al., 1978; Kimball et al., 1979); no binding was observed (Table 1). Similarly, non-cross-β fibrils did not interact with sRAGE; neither collagen nor elastin fibrils immobilized on microtiter wells bound RAGE (not shown). These data lend support to the concept that sRAGE recognizes protein aggregates in the form of β-cross structured amyloid fibrils. The apparently higher affinity of RAGE for freshly prepared Aβ (1-42), compared with Aβ (1-40) (Table 1), is likely to be due to the rapid assembly of Aβ (1-42) into fibrils in aqueous medium (see below). Similarly, unlabelled Aβ (1-42) was a more effective competitor, compared with unlabelled Aβ (1-40), for displacement of ¹²⁵I-sRAGE from immobilized Aβ (1-40) (FIG. 1C); IC₅₀'s were about three-fold higher for Aβ (1-40) compared with Aβ (1-42).

In view of these results, it was surprising that among the amyloidogenic peptides, only Aβ in its soluble form was capable of interacting with sRAGE. An alternative explanation might include the formation of amyloid fibrils derived from Aβ initially present in the random conformation during the course of binding experiments. Consistent with this idea, Aβ is clearly more amyloidogenic than other peptides under the experimental conditions employed (Sipe, 1992). To evaluate this possibility, the formation of amyloid fibrils by Aβ (1-40) in vitro was studied in the presence of sRAGE using the thioflavine T fluorescence assay (LeVine, 1993; Soto and Castano, 1996). In the presence of sRAGE, significant amounts of amyloid were detected even at incubation times as short as 1 hour, and fibrillogenesis was potentiated throughout the time course (FIG. 1E). Enhanced Aβ amyloid formation in vitro occurred at relatively low concentrations of receptor (1:10-1:500 for sRAGE:Aβ monomer molar ratio), and reached a maximum at a molar ratio of 1:50 (FIG. 1F). Experiments were performed under the same conditions using a series of control proteins, including other immunoglobulin superfamily molecules, such as a soluble form of the poliovirus receptor (Gomez et al., 1993) and F(ab′)₂ prepared from nonimmune (IgG), and albumin (FIG. 1G). None of these proteins enhanced Aβ amyloid formation. Consistent with these data, electron microscopic analysis of Aβ (1-40) preparations in the presence of RAGE showed a greater density of fibrils (not shown). RAGE was also found to enhance β-sheet fibril assembly when Aβ(1-42) was used in place of Aβ(1-40), but because of rapid fibril formation with Aβ(1-42) alone, the time scale was considerably compressed.

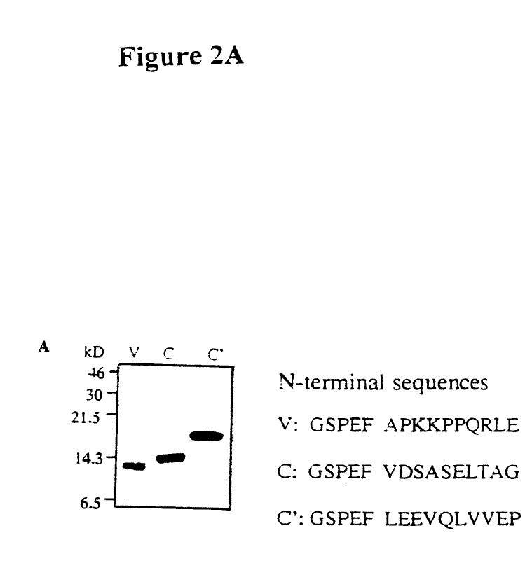

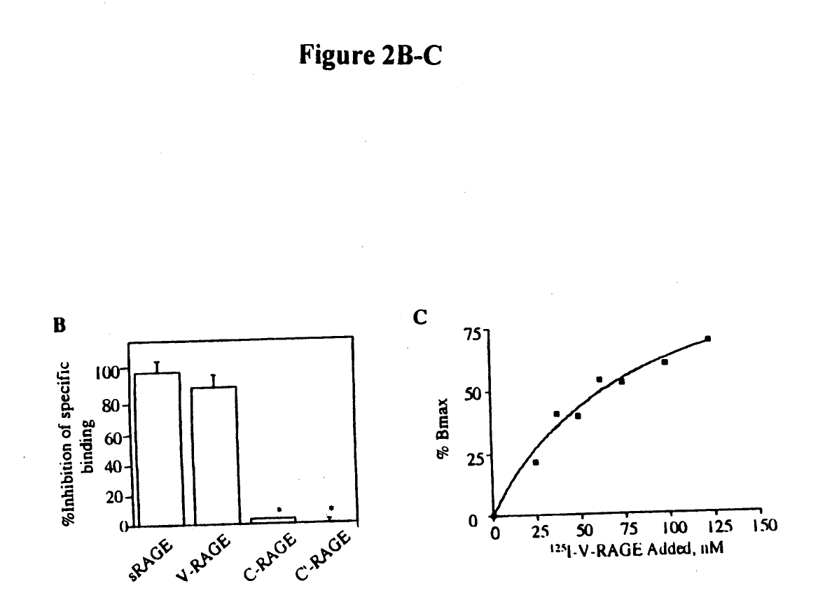

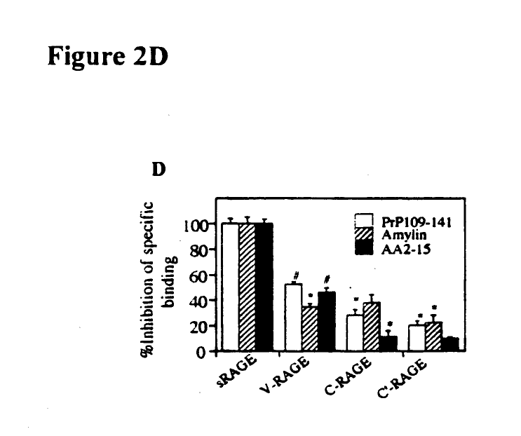

To localize structural determinants in RAGE mediating interaction with fibrils, the extracellular portion of the receptor, comprised of one N-terminal V-type domain followed by two C-type domains (termed C and C′), was further analyzed. Domain-specific fusion proteins with glutathione-S-transferase (GST) were expressed in E. Coli. Following thrombin treatment to remove GST, RAGE domains were purified to homogeneity. By SDS-PAGE, a single band was observed in each case, with Mᵣ'S corresponding to 13 kDa (V; residues 41-126), 16 kDa (C; residues 127-234) and 18 kDa (C′; residues 234-344), respectively, and the amino acid sequence from the N-terminus is indicated (FIG. 2A). Using purified RAGE domains, competitive binding studies were performed with ¹²⁵I-sRAGE and immobilized fibrillar Aβ(1-40); addition of a 50-fold molar excess of unlabelled V-domain blocked binding, whereas C- and C′-domains were without effect (FIG. 2B). Radioligand studies with ¹²⁵I-V-domain displayed binding to fibrillar Aβ(1-40) with K_{d}≈78 nM (FIG. 2C), consistent with a central role in mediating the interaction with Aβ fibrils. Competitive binding experiments were then performed with prion peptide-, amylin- and amyloid A peptide-derived fibrils. Although excess sRAGE (100-fold molar excess) completely blocked binding of ¹²⁵I-sRAGE to these immobilized fibrils, even in the presence of an 100-fold molar excess of V-domain, inhibition of ¹²⁵I-sRAGE-fibril binding was not greater than 40-50% (FIG. 2D). This suggested the possible involvement of other portions of the receptor, in addition to V-domain, in contributing to the interaction with these types of amyloid. Consistent with this idea, addition of excess C-domain also appeared to inhibit, in part, binding of prion peptide- and amylin-derived fibrils, though the C′-domain was without significant effect (FIG. 2D).

### RAGE Binds Aβ Fibrils at the Cell Surface and Activates Signal Transduction Mechanisms Eventuating in NF-kB Activation and DNA Fragmentation

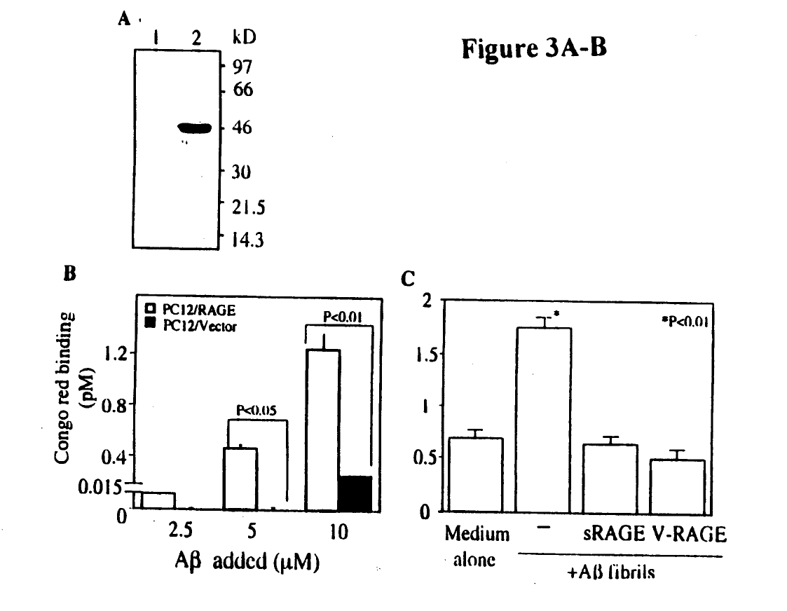

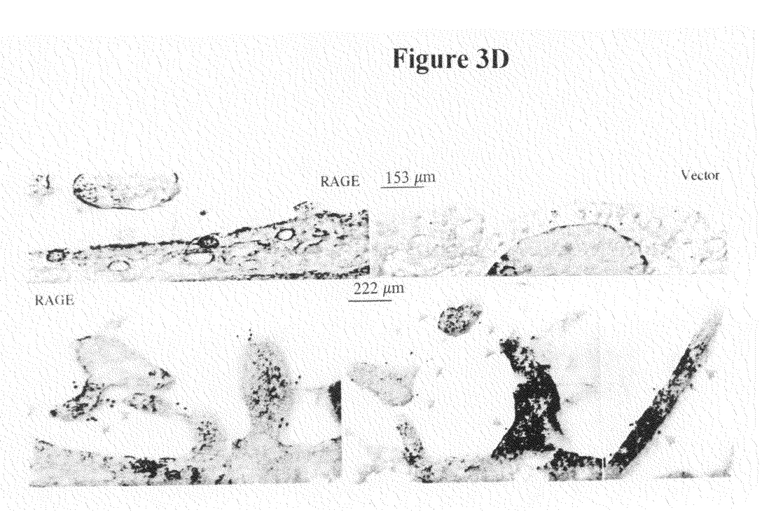

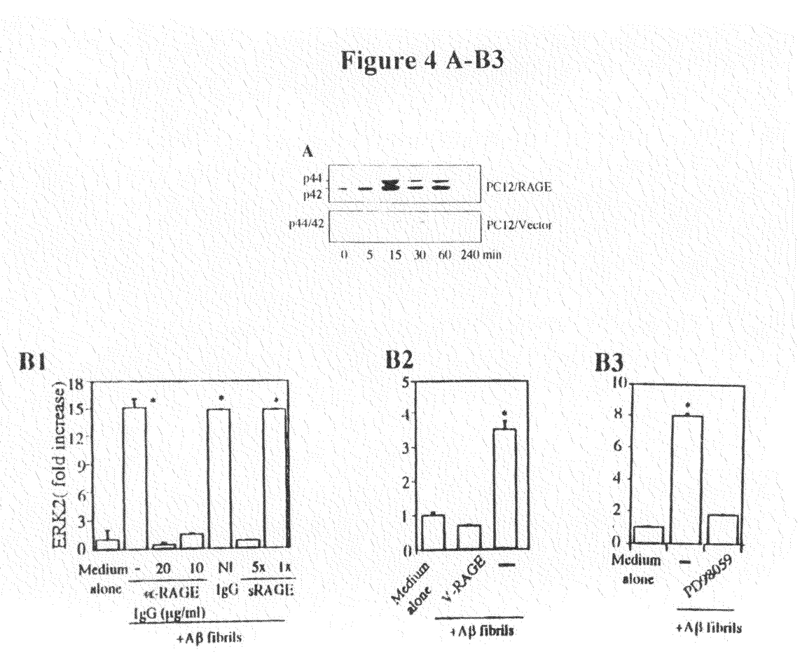

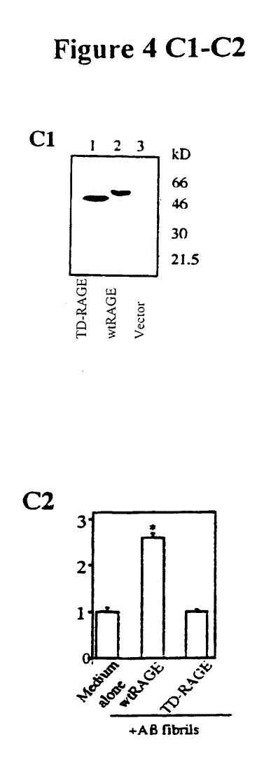

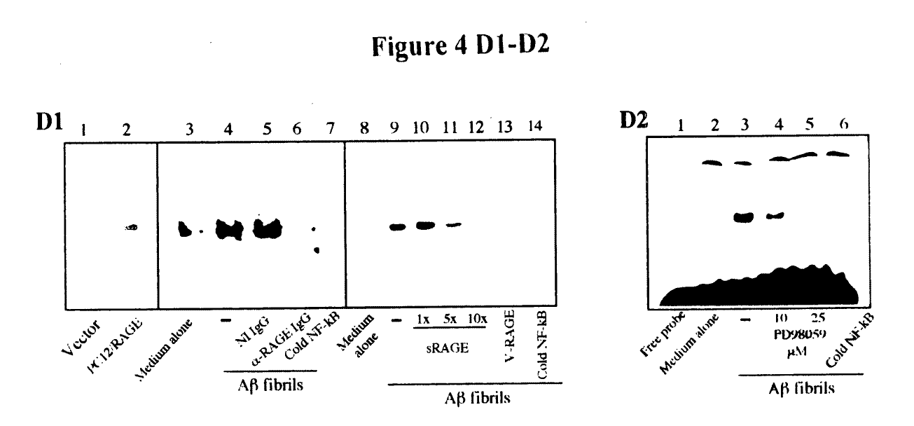

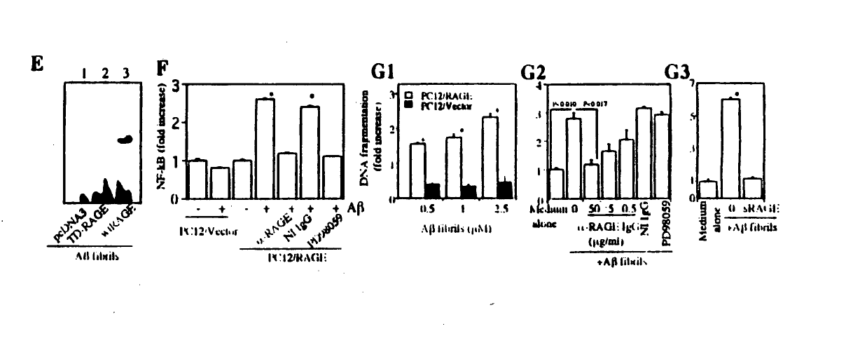

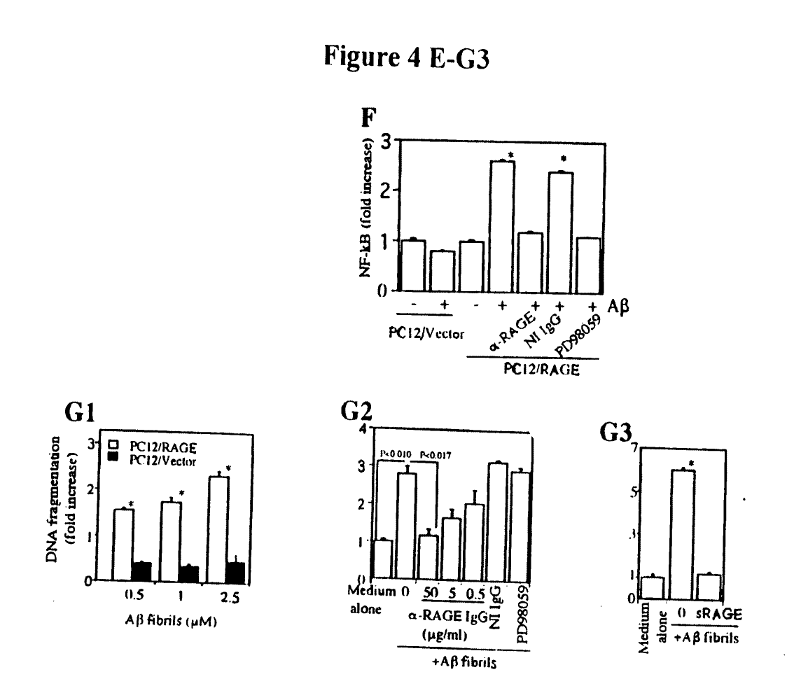

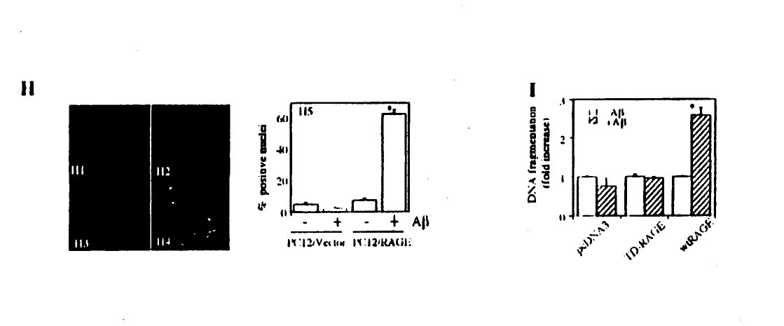

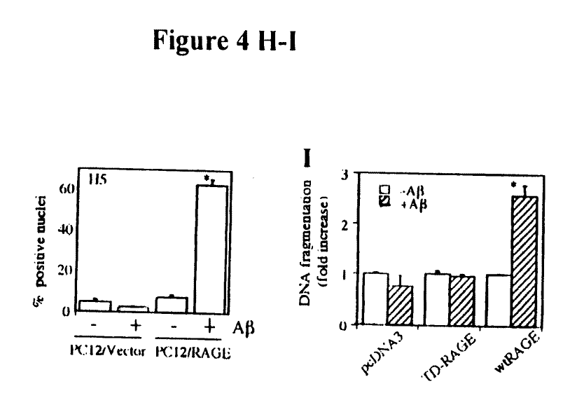

The key issue was to relate RAGE engagement by amyloid fibrils, observed in the purified system (above), to events occurring on the cell surface and their consequences for cellular behavior. Towards this end, a line of PC12 cells with virtually undetectable levels of RAGE was stably-transfected to overexpress wild-type (wt) receptor. PC12 cell-RAGE transfectants (PC12/RAGE) displayed increased total RAGE antigen by immunoblotting (FIG. 3A) and elevated levels of cell surface RAGE by immunocytochemistry, versus mock-transfected controls (not shown). Using an assay in which cell-bound fibrils were quantified by change in the absorbance of Congo red, we first focused on the interaction of PC12/RAGE cells with preformed Aβ(1-40) fibrils. Because of the well-known relative insensitivity of the Congo red assay (Wood et al., 1995), micromolar levels of Aβ (this concentration is derived from the amount of Aβ monomer added at the time fibrils were formed) were required to detect cellular association of fibrils, though functional studies which monitored with greater sensitivity changes in cellular properties due to fibrils were performed using nanomolar levels of Aβ (see below, FIG. 4). Incubation of PC12/RAGE cells with preformed Aβ(1-40) fibrils demonstrated enhanced binding in a dose-dependent manner, versus that observed with PC12/vector (FIG. 3B). Increased binding of Aβfibrils to PC12/vector cells observed at higher levels of added fibrils implicates a role for RAGE-independent binding sites under these conditions, as might be expected for such a complex ligand. However, at lower levels, association of Aβ fibrils with PC12/RAGE cells was RAGE-dependent; binding was blocked by excess sRAGE (at these high concentrations, 10:1 molar ratio of sRAGE:Aβ, the soluble receptor acts as a decoy soaking up Aβ and preventing interaction with cell surface RAGE), as well as by recombinant RAGE V-domain (FIG. 3C). Consistent with the ability of cell surface RAGE to engage Aβ fibrils, electron microscopic analysis of PC12/RAGE cells demonstrated a higher density of surface associated fibrils, compared with vector-transfected control cells (FIG. 3D, upper panels). When RAGE was visualized by immunoelectron microscopy, it was evident that loci in which Aβ fibrils were closely associated with the cell surface corresponded, in part, to sites of RAGE immunoreactivity (FIG. 3D, lower panels). These data support the concept that cell surface RAGE engages Aβ fibrils, potentially enhancing their ability to perturb target cells.

To analyze implications of enhanced Aβ fibril binding for cellular functions in PC12/RAGE cells, activation of the MAP kinase pathway and NF-kB was evaluated. PC12/RAGE cells exposed to Aβfibrils displayed receptor-dependent activation of ERK 1/2, as shown by increased intensity of two closely spaced bands (Mᵣ≈42&44 kDa) immunoreactive with antibody to phosphorylated ERK 1/2, which was not observed to a significant extent with PC12/vector cells (FIG. 4A) ERK 1/2 activation occurred in a time-dependent manner, maximal by 15 min and returning to baseline by 4 hrs. Blockade of cell surface RAGE with increasing amounts of anti-RAGE IgG or sRAGE, suppressed activation of ERK 2 (FIG. 4B1; results of densitometry for ERK 2 are shown in the figure, and similar findings were obtained with ERK 1). Further evidence for the specificity of this pathway was inhibition of ERK 2 activation in the presence of excess soluble RAGE V-domain (FIG. 4B2). The signalling pathway activated by RAGE-Aβ fibril interaction was likely analogous to that previously described for AGE-mediated activation of RAGE (Lander et al., 1997) and Aβ-induced cellular perturbation (Combs et al., 1999), which involves MEK activation of MAP kinases, as shown by its suppression in the presence of the MEK inhibitor PD98059 (FIG. 4B3). To be certain that RAGE was functioning as a signal transducer, rather than simply tethering fibrils with intrinsic bioactivity to the cell surface, experiments were performed with tail-deleted (TD)-RAGE, a truncated form of the receptor comprising the extracellular and transmembrane spanning domains, but lacking the cytosolic tail (Hofmann et al., 1999). Transfection of cultures with pcDNA3/TD-RAGE resulted in expression of RAGE immunoreactive material with Mᵣ≈45 kDa, compared with a band corresponding to Mᵣ≈50 kDa following transfection with pcDNA3/wild-type (wt) RAGE (FIG. 4C1). Expression of TD-RAGE and wtRAGE was comparable in cell lysates (FIG. 4C1) and on the cell surface, and binding studies demonstrated that cultured cells expressing TD-RAGE bound Aβ fibrils comparably to cells transfected to overexpress wtRAGE using the Congo red assay (not shown). Despite the capacity of cells transfected with pcDNA3/TD-RAGE to bind Aβ fibrils, activation of ERK 2 was not observed, compared with cells overexpressing wtRAGE (FIG. 4C2).

As assessed by electrophoretic mobility shift assay (EMSA), expression of RAGE also increased cellular sensitivity to activation of NF-kB in the presence of preformed Aβ(1-40) fibrils compared with PC12/vector controls (FIG. 4D1, lanes 1-2). Incubation of Aβ(1-40) fibrils with PC12/RAGE cells resulted in a strong gel shift band whose appearance was prevented by addition of anti-RAGE IgG (FIG. 4D1, lane 6, compared to nonimmune IgG, lane 5) and was attenuated in the presence of increasing concentrations of sRAGE and RAGE V-domain (FIG. 4D1, lanes 10-13). RAGE-dependent signal transduction mechanisms were mediating Aβ fibril-induced NF-kB activation, as this was blocked by inclusion of PD98059 (FIG. 4D2), and was strikingly diminished in cells overexpressing TD-RAGE, compared with those expressing wtRAGE (FIG. 4E). NF-kB activation triggered by RAGE binding to Aβ fibrils resulted in activation of transcription as shown by increased expression of a luciferase reporter whose expression was driven by four NF-kB sites in PC12/RAGE cells compared with PC12/vector controls (FIG. 4F). Expression of the luciferase reporter in PC12/RAGE cells exposed to Aβwas prevented by anti-RAGE IgG and PD98059, in support of the results described above. These observations are consistent with enhanced expression of genes regulated by NF-kB in Alzheimer's brain, such as heme oxygenase type 1 (HO-1), macrophage-colony stimulating factor (M-CSF) and Interleukin (IL) 6 (Strauss et al., 1992; Smith et al., 1994; Yan et al., 1997).

Another consequence of the interaction of Aβ fibrils with RAGE was induction of DNA fragmentation. Using an ELISA for cytoplasmic histone-associated DNA fragments, PC12/RAGE cells displayed DNA cleavage in the presence of increasing amounts of Aβ fibrils, compared with PC12/vector cells (FIG. 54G1). Blockade of Aβ fibril binding to RAGE with anti-RAGE IgG (FIG. 4G2) or excess sRAGE (FIG. 4G3) prevented DNA fragmentation. Consistent with these data, the TUNEL assay strongly labelled nuclei in PC12/RAGE cells exposed to Aβ fibrils, but not in vector-transfected controls (FIGS. 4H1-5). To be certain that RAGE-dependent mechanisms were responsible for Aβ fibril-induced DNA fragmentation, experiments were performed in transfected neuroblastoma cells using pcDNA3/wtRAGE or pcDNA3/TD-RAGE (FIG. 4I). Neuroblastoma cells expressing wtRAGE in the presence of Aβ fibrils showed DNA fragmentation, whereas under the same conditions, cultures overexpressing similar levels of TD-RAGE did not show DNA fragmentation (FIG. 4I). It was important to determine if the RAGE-dependent signalling pathway causing activation of MAP kinases and NF-kB was distinct from that resulting in DNA fragmentation. Preincubation of PC12/RAGE cells with PD98059 had no effect on Aβ fibril induction of DNA fragmentation (FIG. 4G2), though, under the same conditions, MAP kinase and NF-kB activation were blocked (FIGS. 4B3&4D2). These results show that Aβ fibril binding to RAGE triggers events leading to fragmentation of nuclear DNA, whereas Aβ-RAGE-dependent activation of the MAP kinase pathway engages a distinct set of mechanisms.

### Cell Surface RAGE Binds Amylin and Prion Peptide-Derived Fibrils, and Triggers Cellular Activation

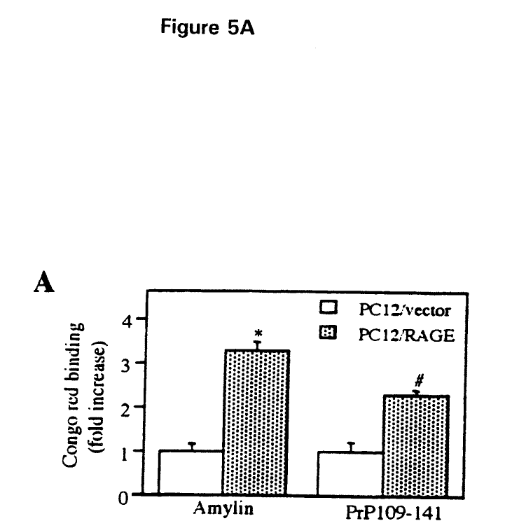

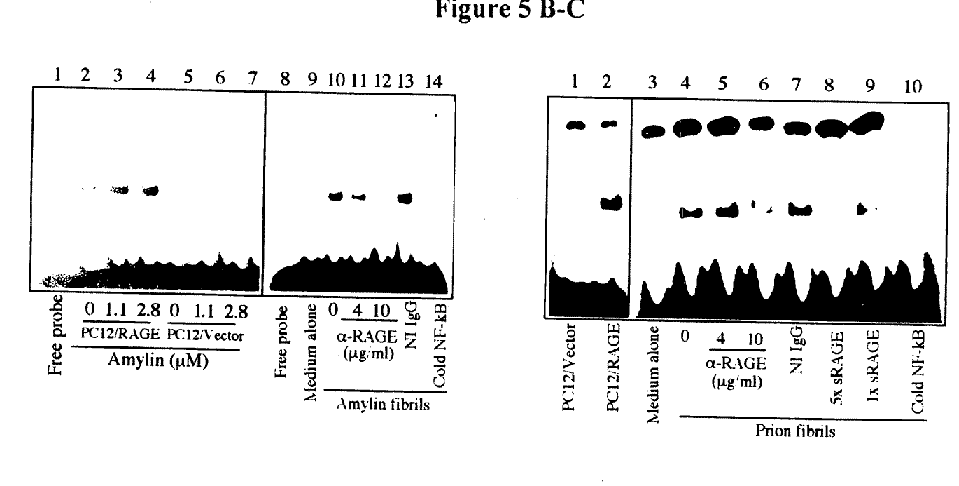

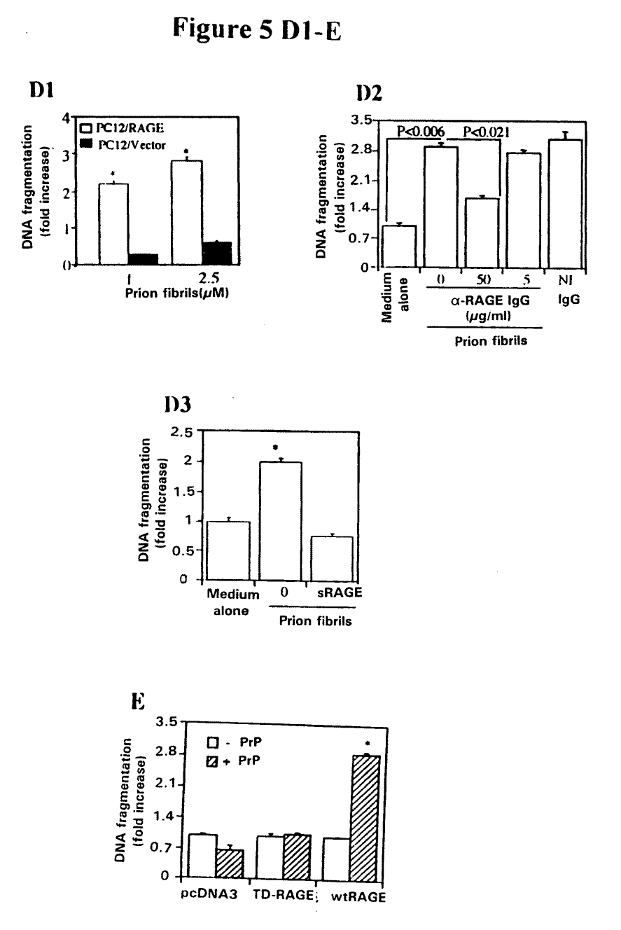

In view of the comparable binding of purified RAGE to fibrillar Aβ and amyloid composed of amylin and prion-derived peptides, it was logical to expect that cell surface RAGE might similarly engage these fibrils. PC12/RAGE cells displayed preferential binding of amylin and prion peptide-derived fibrils, compared with PC12/vector controls (FIG. 5A). The functional implications of this fibril binding included nuclear translocation of NF-kB in PC12/RAGE cells, compared with control cells, following exposure to amylin or prion peptide-derived fibrils (FIG. 5B, compare lanes 2-4 & 5-7; FIG. 5C, compare lanes 1-2). Such NF-kB activation was receptor-dependent, as shown by inhibition in the presence of anti-RAGE IgG (FIG. 5B, lanes 11-12; FIG. 5C, lanes 5-6; nonimmune IgG was without effect, FIG. 5B, lane 13 & FIG. 5C, lane 7) and sRAGE (FIG. 5C, lanes 8-9), and reflected sequence-specific nuclear DNA binding activity, as indicated by inhibition with excess unlabelled NF-kB probe (FIG. 5B, lane 14; FIG. 5C, lane 10), but not unrelated probe (not shown). Evidence of DNA fragmentation was also enhanced in PC12/RAGE cells exposed to prion peptide fibrils, compared with vector-transfected controls, using the ELISA for cytoplasmic histone-associated DNA fragments (FIG. 5D1). Based on the inhibitory effect of anti-RAGE IgG (FIG. 5D2) and excess sRAGE (FIG. 5D3), fibril-induced DNA cleavage required amyloid engagement of the receptor. Exposure of prion peptide-derived fibrils to neuroblastoma cells expressing TD-RAGE did not show increased DNA fragmentation, compared with those expressing full-length receptor (FIG. 5E). DNA fragmentation was also observed with amylin-derived fibrils (not shown). Thus, RAGE serves as a signal transduction receptor mediating the effect of several types of β-sheet fibrils derived from amyloidogenic peptides on target cells. It is important to note that although binding of prion peptide and amylin fibrils to PC12/RAGE cells was only enhanced 2-3-fold, compared with PCl₂/vector cells (FIG. 5A), the functional effects of engaging this receptor were striking, as blockade of RAGE suppressed fibril-dependent NF-kB activation and DNA fragmentation virtually completely (FIG. 5B-E).

Interaction of RAGE with Serum Amyloid A-Derived Fibrils: Effect on Cellular Properties In Vitro and In Vivo

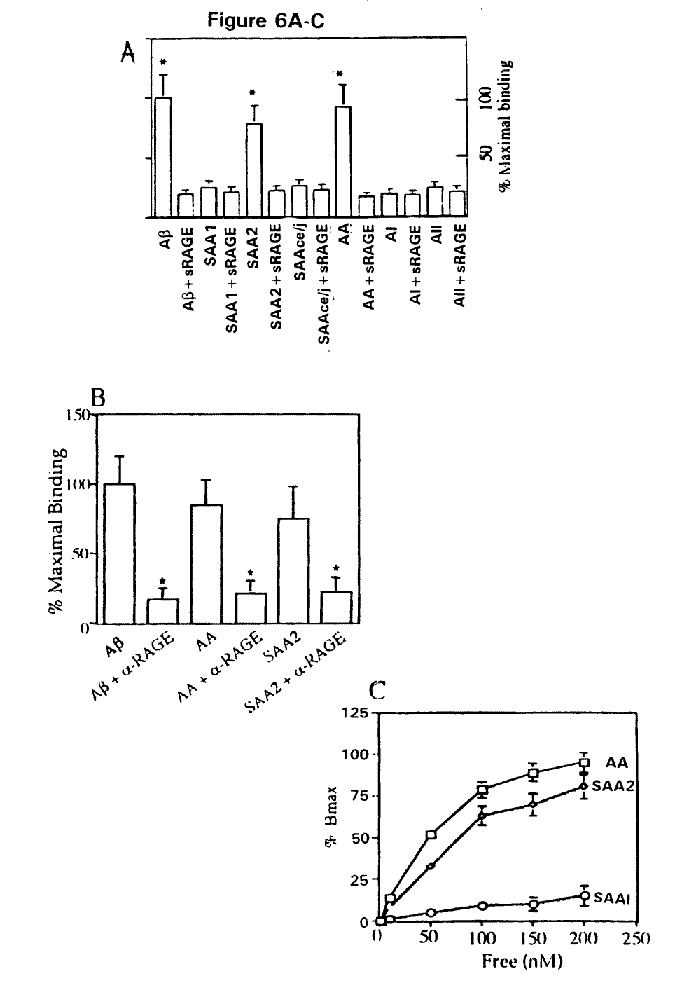

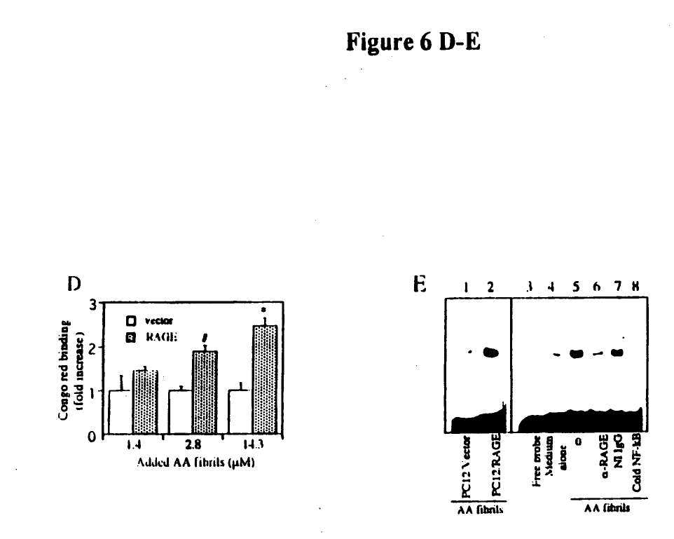

A critical step in extrapolating the concept of RAGE as a receptor for multiple kinds of amyloid was to perform experiments with β-sheet fibrils assembled from a full-length polypeptide. Assessment of the potential binding of RAGE to fibrils derived from serum amyloid A (SAA) was especially attractive in view of the availability of in vitro and in vivo model systems to test the functional consequences of such an interaction. Radioligand binding studies were performed with ¹²⁵I-sRAGE added to wells with adsorbed apoSAA1 (the isoform not prone to fibril formation), apoSAA2 (the isoform prone to fibril formation), amyloid A fibrils (isolated from murine splenic tissue), apoSAAce/j (non-fibrillogenic), as well as other lipoproteins (apoA-I or apoA-II)(FIG. 6A) (Sipe et al., 1993; Kindy and Rader, 1998; Shiroo et al., 1998). Binding of ¹²⁵I-sRAGE to SAA2 and amyloid A fibrils was observed, though no significant interaction was seen with apoSAAce/j or apoSAA1. Furthermore, ¹²⁵I-sRAGE did not interact with apoA-I or apoA-II, indicating that it was not nonspecifically binding to hydrophobic polypeptides. Selectivity of binding in this assay was further tested by inhibition in the presence of excess unlabelled sRAGE (FIG. 6A) or anti-RAGE IgG (FIG. 6B). Experiments in which ¹²⁵I-sRAGE was incubated in wells with fibrillar apoSAA2 or amyloid A fibrils demonstrated dose-dependent binding with k_{d}'s of ≈72 nM and ≈60 nM, respectively (FIG. 6C); this was virtually identical to the binding of ¹²⁵I-sRAGE to Aβ and amyloid A peptide (2-15)-derived fibrils (FIGS. 1A-B, D3). No saturable binding of ¹²⁵I-sRAGE to adsorbed apoSAA1 was observed (FIG. 6C). As implied by these data with purified RAGE, amyloid A fibrils displayed enhanced binding to PC12/RAGE cells compared with PC12/vector controls (FIG. 6D). In addition, PC12/RAGE cells incubated with amyloid A fibrils showed consequences of RAGE-fibril interaction, for example, enhanced activation of NF-kB, compared with vector-transfected control cultures (FIG. 6E, compare lanes 1-2). Addition of blocking antibody to RAGE strongly suppressed amyloid A fibril-induced NF-kB activation, compared with nonimmune IgG (FIG. 6E, lanes 6-7), consistent with a central role for RAGE in amyloid A-fibril-induced cellular perturbation (see below).

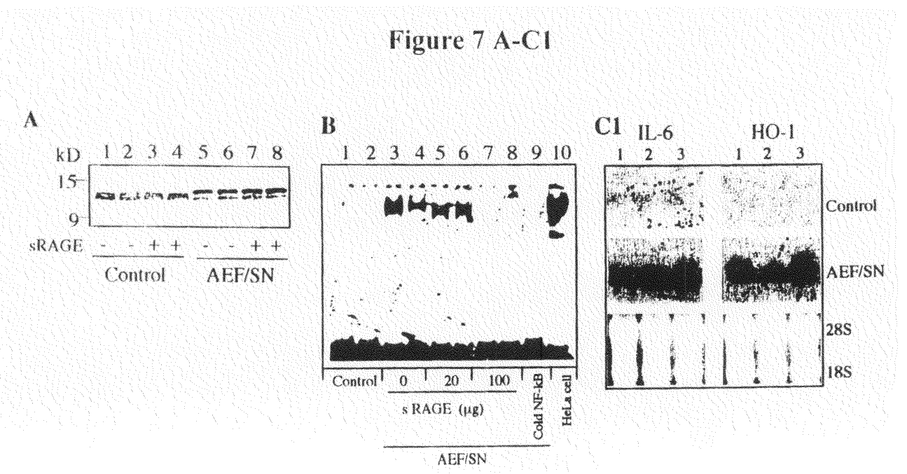

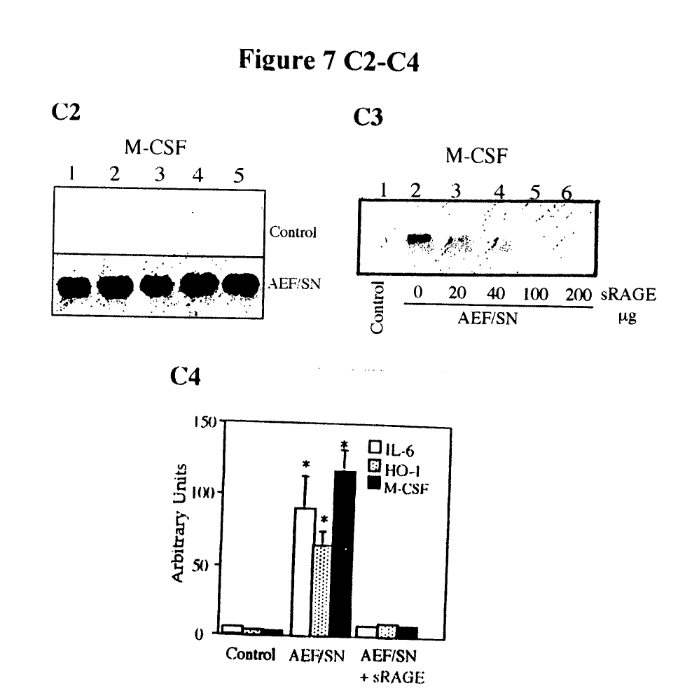

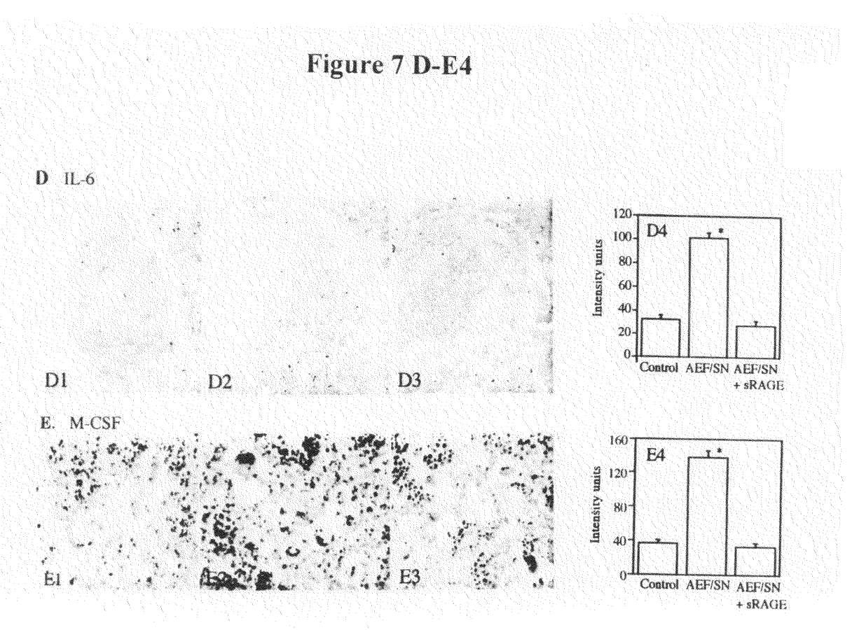

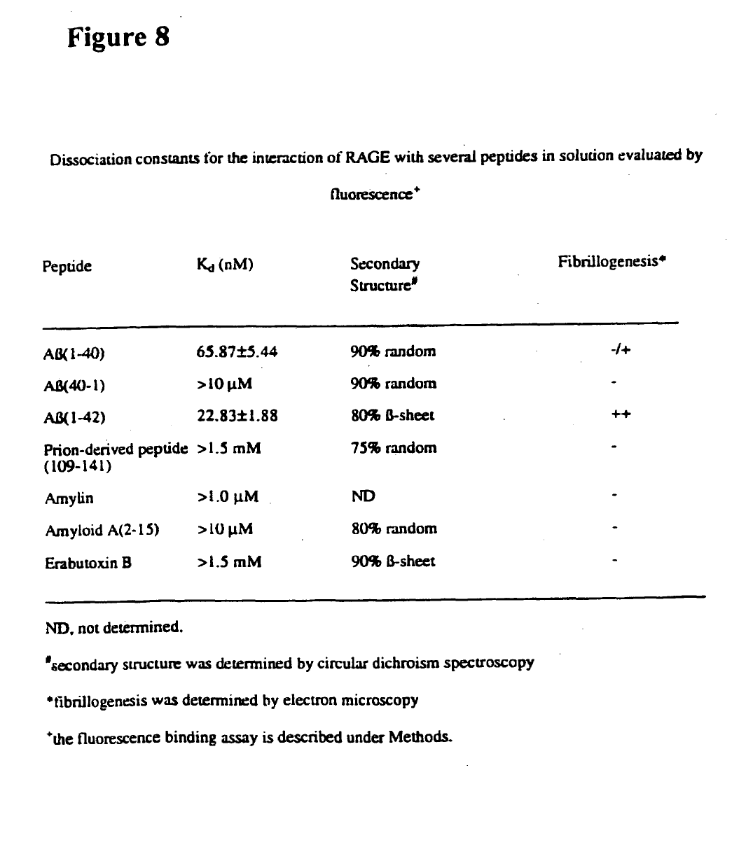

A critical test of our concept concerning RAGE as a receptor for β-sheet fibrils was to use a murine model of systemic amyloidosis. In this model, C57BL6 mice are injected with amyloid enhancing factor (AEF) and silver nitrate (SN) over five days. Rapid accumulation of splenic amyloid displays the acute consequences of a β-sheet-rich fibril environment (Kisilevsky et al., 1995; Kindy and Rader, 1998). Immunoblotting demonstrated increased levels of SAA in plasma of mice receiving AEF/SN, compared with untreated animals (FIG. 7A). This was accompanied by evidence of cellular perturbation in the spleen as assessed by activation of NF-kB and target genes, including IL-6, HO-1, and M-CSF (see below). NF-kB activation was studied in AEF/SN-treated mice by EMSA with ³²P-labelled NF-kB consensus probe (FIG. 7B); although nuclear extracts prepared from spleens of control mice showed only a weak/absent gel shift band (lanes 1-2), the intensity of this band increased dramatically with AEF/SN treatment (lanes 3-4). This nuclear binding activity was specific for NF-kB, as it was blocked by inclusion of excess unlabelled NF-kB probe (lane 9). Levels of IL-6, HO-1, and M-CSF transcripts also increased in mice subjected to the AEF/SN protocol (FIGS. 7C1-2,4). Consistent with these data, splenic IL-6 antigen was strongly elevated in AEF/SN-treated mice, compared with samples from untreated control animals (FIGS. 7D1,2&4). Also, strikingly enhanced staining for M-CSF in splenic mononuclear phagocytes was observed in mice treated with AEF/SN (FIGS. 7E1,2&4). Taken together with the accumulation of splenic amyloid in AEF/SN-treated mice, compared with controls (FIG. 7F), these data show a strong association between increased tissue amyloid burden and cellular stress.

The relevance of RAGE biology to this model of systemic amyloidosis was demonstrated by analyzing RAGE expression in the spleen. Northern analysis showed an increase in RAGE transcripts (≈3.2-fold by densitometry) in AEF/SN-treated mice (FIGS. 7G1-2). RAGE antigen in the spleen also increased in AEF/SN mice (FIGS. 7H2), compared with untreated controls (FIGS. 7H1; ≈3.5-fold by densitometry, 7H4). The distribution of endogenous RAGE in AEF/SN mice overlapped closely with that of amyloid A in the spleen (FIG. 7H6; no amyloid A is seen in untreated controls, 7H5), consistent with the likelihood that RAGE interaction with amyloid A fibrils occurred in vivo. If this was true, we reasoned that administration of sRAGE (at concentrations which would locally probably achieve a molar excess of soluble receptor to that of fibrils) might blunt the cellular effects of amyloid A fibrils, potentially by preventing their interaction with and activation of cell surface RAGE. Recombinant sRAGE was injected once daily (intraperitoneally) from days −1 to 4 (with respect to AEF/SN treatment). Although levels of apoSAA in the plasma remained comparably elevated in AEF/SN-treated mice, whether treated with vehicle or sRAGE (FIG. 7A, compare lanes 5-6 to 7-8), suppression of NF-kB activation was observed; the gel shift band in AEF/SN mice was undetectable at the 100_g dose of sRAGE (FIG. 7B, compare lanes 3-4 to 7-8). In parallel, splenic M-CSF (FIGS. 7C3-4), HO-1 (FIG. 7C4) and IL-6 (FIG. 7C4) transcripts were strikingly diminished in samples from AEF/SN mice treated with sRAGE reaching levels in control animals (FIG. 7C4). Immunostaining of splenic tissue from AEF/SN mice administered sRAGE also showed a striking decrease in IL-6 and M-CSF antigen (FIGS. 7D3-4, 7E3-4).

Consistent with the possibility that sRAGE at the concentrations administered prevented amyloid A fibrils from interacting with cell surface RAGE in AEF/SN mice, immunostaining of splenic tissue from AEF/SN+sRAGE mice showed an increase in RAGE staining (FIG. 7H3; 7H1 shows RAGE staining in control mice) which closely overlapped the expression of endogenous RAGE (FIG. 7H2) and deposited amyloid (FIG. 7H6; compare with control animal, 7H5). The likelihood that the latter increase in RAGE antigen was due to the injected sRAGE, rather than enhanced expression of endogenous receptor, was strengthened by the observed suppression of RAGE transcripts in AEF/SN mice receiving sRAGE down to levels observed in control (non-AEF/SN-treated) animals (FIGS. 7G1-2). Furthermore, immunoprecipitation of plasma from AEF/SN mice treated with sRAGE using anti-RAGE IgG, followed by immunoblotting of precipitated material with anti-aposAA IgG, showed two immunoreactive bands (≈14 and ≈9 kDa) not observed when preimmune IgG was used in place of anti-RAGE IgG (FIG. 7I1, lanes 1-2). Conversely, immunoprecipitation of plasma from AEF/SN+sRAGE mice with antibody to apoSAA, followed by immunoblotting of precipitated material with anti-RAGE IgG, displayed RAGE immunoreactive material (FIG. 7I2, lane 1) which comigrated with purified sRAGE (lane 3). These data indicated the presence of SAA-sRAGE complex in plasma of AEF/SN mice treated with sRAGE. Importantly, apoSAA-sRAGE complex was not detected on HDL particles (not shown), indicating that the association was not likely to be through circulating lipoproteins.

These observations suggested the possibility that sRAGE might not only bind to amyloid A fibrils, intercepting their association with cell surface RAGE, but that soluble receptor might also interact with apoSAA as it assembles into nascent amyloid fibrils thereby impacting on the splenic burden of amyloid A. Dose-dependent suppression of splenic amyloid burden (up to 60%) was observed in sRAGE-treated AEF/SN mice, compared with animals receiving vehicle (mouse serum albumin) alone (FIG. 7F). Although the mechanism of sRAGE-mediated decrease in splenic amyloid remains to be determined, it is possible that sRAGE-mediated inhibition of fibril anchoring to the cell surface promotes local clearance of the amyloid. Consistent with the close interaction of sRAGE with nascent amyloid was the presence of a more rapidly migrating apoSAA-immunoreactive band (Mᵣ≈9 kDa) in the sRAGE-amyloid A complex (FIG. 7I1, lane 1), in addition to the more slowly migrating band corresponding to native/plasma apoSAA (Mᵣ≈14 kDa) (FIG. 7I1, lanes 1&3). Cleavage of intact apoSAA2 in the tissue, presumably following dissociation of SAA2. from HDL, is an integral part of fibrillogenesis (Levin et al., 1972). Thus, we propose that sRAGE binds to amyloid A in nascent fibrils promoting, in part, clearance from the splenic microenvironment.

Administration of fragments [F(ab′)₂] prepared from blocking polyclonal antibody to RAGE to mice undergoing treatment with amyloid enhancing factor/silver nitrate resulted in suppression of markers of cellular stress and amyloid accumulation in the spleen similarly to what was observed in animals treated with sRAGE (data not shown).

### Discussion

Several properties of RAGE make it a particularly suitable candidate for amplifying the pathogenic effects of Aβ. RAGE is expressed at high levels on a range of cells in AD, including affected neurons, microglia, astrocytes and cerebral vasculature (Yan et al., 1996) (and unpublished observations, Yan, Stern and Schmidt, 1999). Furthermore, interaction of RAGE with Aβ upregulates expression of the receptor (not shown) by a mechanism similar to that observed previously with lipopolysaccharide and tumor necrosis factor; activation of transcription at two functional NF-kB sites in the RAGE promoter causes increased levels of receptor (Li and Schmidt, 1997). Most importantly, in the presence of nanomolar levels of Aβ, RAGE-bearing cells display increased susceptibility to modulation of cellular properties with respect to activation of NF-kB, expression of IL-6, HO-1 and M-CSF, and induction of DNA fragmentation (Yan et al., 1996; Yan et al., 1997). However, a puzzle concerning Aβ-RAGE interaction was that soluble Aβ, presumably in random conformation and known for its lack of toxic effects (Pike et al., 1993; Yankner, 1996), appeared able to bind RAGE and activate target cells. Findings in the current paper provide an explanation for this apparent paradox and broaden the perspective on RAGE as a receptor mediating cellular interactions with β-sheet fibrils. Increased fibrillogenesis in the presence of low concentrations of RAGE suggests that the receptor itself promotes fibril formation on the cell surface, with its potential substrates being Aβ monomer, dimers or diffusible nonfibrillar assemblies (Roher et al., 1996; Lambert et al., 1998). Once bound to RAGE, signal transduction mechanisms are triggered causing activation of kinase cascades, including the MAP kinase pathway leading to nuclear translocation of NF-kB, as has been described in other studies of Aβ-cellular interactions (Behl et al., 1994; Akama et al., 1998; Combs et al., 1999). In contrast, high concentrations of administered sRAGE (several-fold molar excess of soluble receptor to Aβ) have a cytoprotective effect, mopping up Aβ and preventing its interaction with the cell surface.

### RAGE as a Receptor for Cross-β Fibrils

The formation of amyloid is basically a problem of protein folding, whereby a mainly random coil/a-helical soluble protein becomes aggregated adopting a β-pleated sheet conformation (Kelly, 1996; Lansbury, 1999; Soto, 1999). Amyloid formation proceeds by hydrophobic interactions among conformationally altered amyloidogenic intermediates, which become structurally organized in a β-sheet conformation upon peptide interaction, forming small oligomers, which are the precursors of the cross-β amyloid fibrils. The propensity of a particular protein to undergo this transition depends on the relative stabilities of the native state and the β-sheet rich intermediate, and the energy barrier between the states. Several environmental (pH, metal ions, reactive oxygen species, etc) and protein factors (apolipoprotein E, amyloid P component, a₁-antichymotrypsin, etc) have been shown to enhance amyloidogenesis, possibly by decreasing the activation energy barrier or by promoting nucleus formation (Soto, 1999). In the present study, we show that RAGE appears to bind specifically to cross-β structured amyloid fibrils regardless of the protein/peptide subunit involved. The amyloidogenic proteins in solution did not bind RAGE with the exception of Aβ. Furthermore, no interaction of RAGE was detected with the unrelated polypeptide erabutoxin B, which adopts a non-amyloid β-sheet rich structure in solution, or other unrelated peptides bearing a similar degree of hydrophobicity to Aβ. Finally, protein aggregates not ordered in a cross-β conformation, such as collagen and elastin, were also unable to bind RAGE. There are two potential explanations for the observation that only Aβ in the soluble state was capable of interacting with RAGE. First is that in addition to the conformation/aggregation-specific binding of RAGE to fibrils, there is a sequence-specific binding site for monomeric Aβ. Second, and probably more likely, is that during the course of the incubation period, the originally soluble Aβ peptide becomes aggregated forming oligomeric β-sheet structures and short amyloid fibrils. The latter is supported by experiments showing that even at short incubation times Aβ formed detectable thioflavine T positive fibrils. Moreover, the presence of RAGE at concentrations similar to those used for the binding experiments significantly promoted Aβ fibrillogenesis in vitro. These data are consistent with the apparently higher affinity of RAGE for soluble Aβ(1-42) compared with Aβ(1-40); Aβ(1-42) more rapidly assembles into fibrils which bind avidly to RAGE. Thus, under our experimental conditions, cell surface RAGE seems to play three different, but related, roles with respect to Aβ: a) serving as an anchor for the interaction of fibrils with the cell surface; b) mediating amyloid-dependent cellular activation by triggering signal transduction pathways; and, c) enhancing amyloid fibril formation in the immediate environment of the cell surface. This situation contrasts with the cytoprotective effect of sRAGE, when present in molar excess compared with Aβ or SAA, which prevents interaction of fibrillar material with cell surface RAGE.

### Common Denominators of Fibrillar Patholoaies

Fibrillar pathologies, such as AD and systemic amyloidosis, have been considered to result principally from accumulated debris in the form of fibrils encroaching on normal structures. Recent data concerning the cellular effects of amyloid fibrils has forced a re-evaluation of this concept, as there is much evidence that an active cellular response to Aβ is integral to the evolving pathology. In this context, the identification of RAGE as a signal transduction receptor for b-sheet fibrils demonstrates a means through which fibril formation changes the biologic signature of the amyloid for cellular interactions. These observations suggest a possible basis underlying similarities in the effects of β-sheet fibrils observed in vitro and pathologic findings in amyloidoses due to fibrils of different composition (Forloni et al., 1996; Mattson and Goodman, 1995; Yankner, 1996). For example, in dialysis-related amyloidosis, the amyloid deposited in joints is composed, in large part, of AGE adducts of β₂-microglobulin (Miyata et al., 1993). AGE-β₂-microglobulin isolated from these patients causes RAGE-dependent activation of mononuclear phagocytes (whereas native β₂-microglobulin does not), analogous to what we have observed with Aβ (Miyata et al., 1996; Yan et al., 1996). These data concerning the outcome of RAGE-β-sheet fibril interaction can be contrasted with that following Aβ binding to the macrophage scavenger receptor; the latter much more effectively internalizes and degrades Aβ than does RAGE (Khoury et al., 1996; Paresce et al., 1996; Mackic et al., 1998). Our results support a role for RAGE in propagating cellular dysfunction in AD, and, potentially, in other amyloidoses as well.

Whereas mutations in βAPP and the presenilins modulate processing of βAPP in familial AD, and alleles of apoE, a₂-macroglobulin, and LRP appear to confer increased risk of sporadic AD (Hardy, 1997; Lendon et al., 1997; Kang et al., 1997; Roses, 1998; Liao et al., 1998; Blacker et al., 1998), we speculate that elevated expression of RAGE in either form of AD functions as a progression factor sustaining cellular perturbation in the Aβ-rich environment. The outcome of Aβ-RAGE-mediated cellular stimulation probably varies in a cell-type specific manner; for example, we hypothesize that Aβ-RAGE interaction on neurons in vivo causes cell stress eventuating in a cytotoxic outcome, whereas Aβ-RAGE activation of microglia causes cell stress, one manifestation of which is M-CSF expression (Yan et al., 1997). The latter enhances macrophage survival and induces their proliferation (Stanley et al., 1997), resulting in a quite different outcome for RAGE-induced activation in these two cell types. Analysis of the effects of RAGE in transgenic models, using as a starting point, for example, mice overexpressing mutant forms of βAPP to create an Aβ-rich environment, should provide the most concrete evidence to further elucidate the role of this receptor-dependent pathway in the pathogenesis of chronic cellular dysfunction in disorders with β-sheet fibrillar pathology.

### Second Series of Experiments

Accumulation of fibrils composed of amyloid A in tissue resulting in displacement of normal structures and cellular dysfunction is the characteristic feature of systemic amyloidoses. Here we show that RAGE, a multiligand immounoglobulin superfamily cell surface molecule, is a receptor for the amyloidogenic form of serum amyloid A. Interactions between RAGE and amyloid A induced cellular perturbation. In a mouse model, amyloid A accumulation, evidence of cell stress and expression of RAGE were closely linked. Antagonizing RAGE suppressed cell stress and amyloid deposition in mouse spleens. These data indicate that RAGE is a potential target for inhibiting accumulation of amyloid A and for limiting cellular dysfunction induced by amyloid A. The accumulation of extracellular β-sheet fibrils is the hallmark of a diverse class of disorders called amyloidosis¹⁻³ Whether composed of subunits derived from serum amyloid A, transthyretin, immunoglobulin chains or other proteins/protein fragments (amyloid β-peptide, prion protein and so on), deposits of fibrillar material inexorably expand and are associated with dysfunction of surrounding parenchymal cells and vasculature. For example, in system reactive amyloidosis, a sustained inflammatory challenge (regardless of etiology) substantially increases plasma levels of serum amyloid A (SAA). Amyloid A fibrils become deposited widely in the tissues, causing symptoms such as eventual splenic and renal insufficiency¹⁻³. Several studies have emphasized the contribution of polypeptides associated with amyloid A, such as apolipoprotein E (refs. 4-7), serum amyloid P component⁸⁻⁹, and proteoglycans in modulating serum amyloid deposition. Given the close association of amyloid fibrils with cellular elements, such as mononuclear phagocytes, and the recently noted increased levels of tumor necrosis factor (TNF)-α and macrophage colony-stimulating factor (M-CSF) in systemic amyloidosis (amyloid A)¹¹, local cellular activation might contribute to the pathogenesis of amyloidosis. Specifically, interaction of amyloid A fibrils with a cell surface binding site/receptor (for example, one induced on mononuclear phagocytes associated with fibrillar lesions), might alter the local environment to cause cellular dysfunction and to be more conductive for amyloid formation.

Here RAGE (receptor for advanced glycation end-products; Genome Database designation, AGER), a multiligand receptor in the immunoglobulin superfamilyl¹²⁻¹⁴ bound with nanomolar affinity to amyloid A, as well as the mouse isoform of SAA (SAA1.1) most prone to fibrillogenesis^{1,15-18}. Tissue samples from patient-derived and experimentally induced reactive amyloid A amyloidosis demonstrated increased expression of RAGE, and in vitro studies showed amyloid-A induced, RAGE-dependent activation of a mononuclear phagocyte cell line. Blockade of RAGE in a mouse model of systemic reactive amyloidosis suppressed most amyloid accumulation and evidence of cellular perturbation. These data support the possibility of a previously unknown function for a cell surface receptor in the pathogenesis of systemic amyloidosis, and indicate the potential future therapeutic utility of targeting RAGE in amyloidoses.

### RAGE Expression is Enhanced in Systemic Amyloidosis

Splenic tissue from a patient with systemic reactive (amyloid A) amyloidosis showed increased immunoreactive RAGE antigen (FIG. 9a) in a distribution overlapping, at least in part, that of deposited amyloid A (FIG. 9b; Congo red staining showed these deposits of immunoreactive amyloid A contained fibrils, and there was no amyloid A in normal spleen; data not shown). Amyloid deposits have a characteristic appearance (FIG. 9b, inset). Cells most prominently expressing RAGE (FIG. 9c) in the amyloid-laden spleen were of mononuclear phagocyte origin, as shown by double staining with antibody against CD14 (FIG. 9d). Such amyloid-laden spleens also had cells (most likely monocytes/macrophages) strongly expressing the M-CSF antigen (FIG. 9e). There was similarly increased expression of interleukin (IL)-6 in splenic tissue with deposited amyloid A (data not shown). In contrast, splenic tissue from an age-matched normal individual, with no detectable deposited amyloid A (data not shown), had low levels of expression of RAGE (FIG. 9) and M-CSF (FIG. 9g).

### Interaction of Amyloid A Amyloid and RAGE

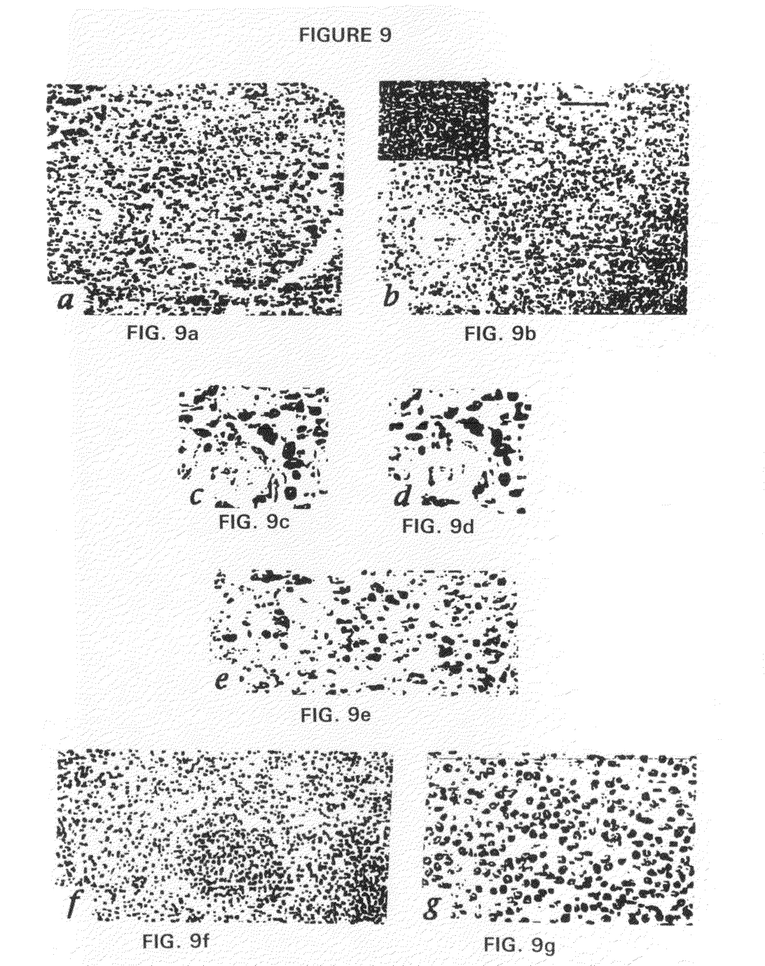

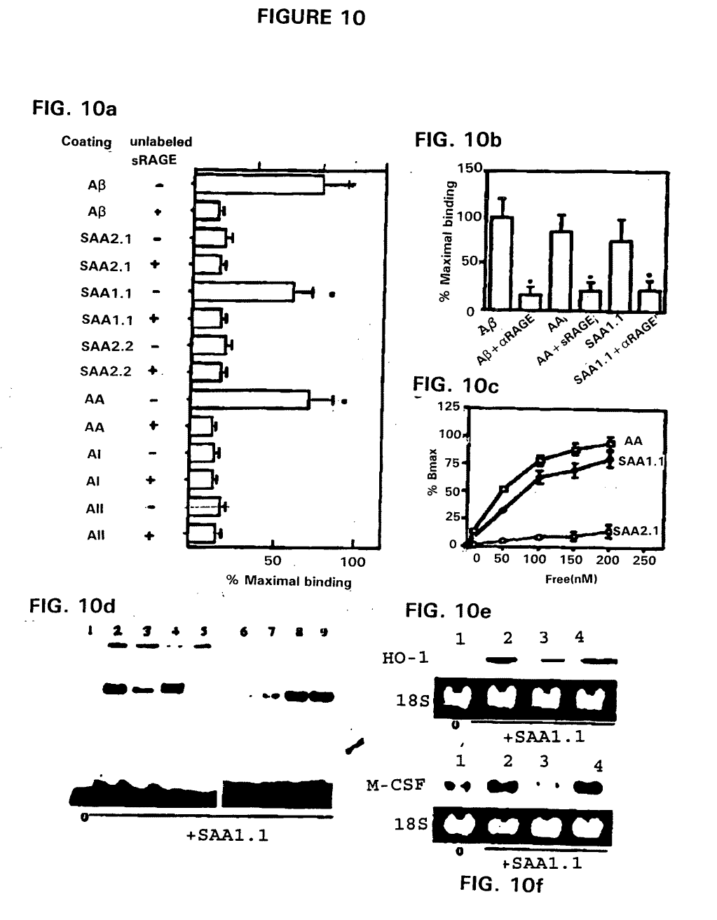

Given the association of RAGE with mononuclear phagocyte activation described above, and the multiligand character of the receptor¹²⁻¹⁴, we investigated the possibility of a direct interaction of amyloid A amyloid with RAGE. Mouse SAA1.1 is the isoform prone to fibril formation, whereas SAA2.1, SAA2.2 and other apolipoproteins such as AI and AII are not^{1,15-17}. We did radioligand binding studies with microtiter wells and absorbed mouse SAA2.1 or SAA2.2, SAA1.1, amyloid A fibrils (isolated from mouse splenic tissue) or other apolipoproteins (AI or AII). After blockade of excess binding sites, wells were incubated with ²⁵¹l-s RAGE (soluble RAGE), a radioiodinated form of the receptor composed of only the extracellular domain^{12,13,19}. There is specific binding of ¹²⁵I-sRAGE to amyloid 5-protein in this assay¹⁴, providing a positive control for our studies here with SAA isoforms. ¹²⁵I-sRAGE bound to SAA1.1 and amyloid A fibrils, although there was no interaction with SAA2.1 or SAA2.2 (FIG. 10a). Furthermore, ¹²⁵I-sRAGE did not interact with AI or AII, indicating that it was not nonspecifically binding to hydrophobic polypeptides. We further tested the selectivity of binding in this assay using inhibition in the presence of excess unlabeled sRAGE (FIG. 10a) or antibody again RAGE (FIG. 10b). Experiments in which ¹²⁵I-sRAGE was incubated in wells with fibrillar SAA1.1 or amyloid A showed dose-dependent binding with K_{d} values of about 73 nM and 60 nM, respectively (FIG. 10c). There was no saturable binding of ¹²⁵I-sRAGE to adsorbed SAA2.1 (FIG. 10c).

These data indicated the possibility that RAGE might be a cellular target for amyloid A or SAA1.1. Because of the close relationship between mononuclear phagocytes bearing RAGE and amyloid A in the spleen (FIG. 9), we focused our attention on cells of monocyte origin. The established line of BV-2 cells²⁰ provides a model system for transformed mouse mononuclear phagocytes containing RAGE, and show RAGE-dependent responses^{13,21}. Incubation of BV-2 cells with SAA1.1 fibril resulted in nuclear translocation of the transcription factor NF-κB (FIG. 10d, lane 2), compared with results in untreated controls (FIG. 10d, lane 1), as assessed by electrophoretic mobility shift assay (EMSA) with a ¹²P-labeled consensus NF-kB probe. Similarly, BV-2 cultures exposed to fibrillogenic amyloid A demonstrated NF-kB activation. The appearance of the gel-shift band in nuclear extracts of BV-2 cells incubated with SAA1.1 reflected sequence specific binding, as shown by inhibition in the presence of NF-kB (FIG. 10d, lane 5). The essential involvement of interaction between RAGE and amyloid A was shown by decreased intensity of the gel shift band in cultures exposed to blocking antibody against RAGE F(ab′)₂ compared with no effect using the same concentration of non-immune F(ab′)₂ (FIG. 10d, lanes 3 and 4, respectively). RAGE was functioning as a signal transduction receptor, rather than simply tethering toxic fibrillar material to the cell surface, as shown by studies with a dominant negative form of the receptor lacking the cytosolic tail¹³. Although dominant negative RAGE binds ligands, its expression prevents RAGE-dependent signal transduction, even in cells with wild-type RAGE, such as BV-2 cells¹³. Transfection of BV-2 cells to overexpress dominant negative RAGE resulted in suppression of SAA1.1-dependent NF-κB activation (FIG. 10d, lanes 6 and 7) compared with cells transfected with vector alone (FIG. 10d, lanes 8 and 9).

Three well-recognized target genes for NF-kB in settings of acute stress include heme oxygenase type 1 (HO-1), IL-6 and M-CSF (ref. 22). Incubation of BV-2 cells with SAA1.1 increased expression of transcripts for HO-1 (FIG. 10e, lane 2). Inclusion of blocking antibody against RAGE F(ab′)₂ with BV-2 cells incubated with SAA1.1 mostly suppressed the induction of transcripts for HO-1 and M-CSF (FIG. 10e and f, lane 3), whereas nonimmune F(ab′)₂ (FIGS. 10e and f, lane 4) had no effect. We obtained similar results for the induction of IL-6 transcript by SAA1.1 with BV-cells (data not shown).

### Effect of RAGE Blockade on Cell Activation and Amyloid Deposition

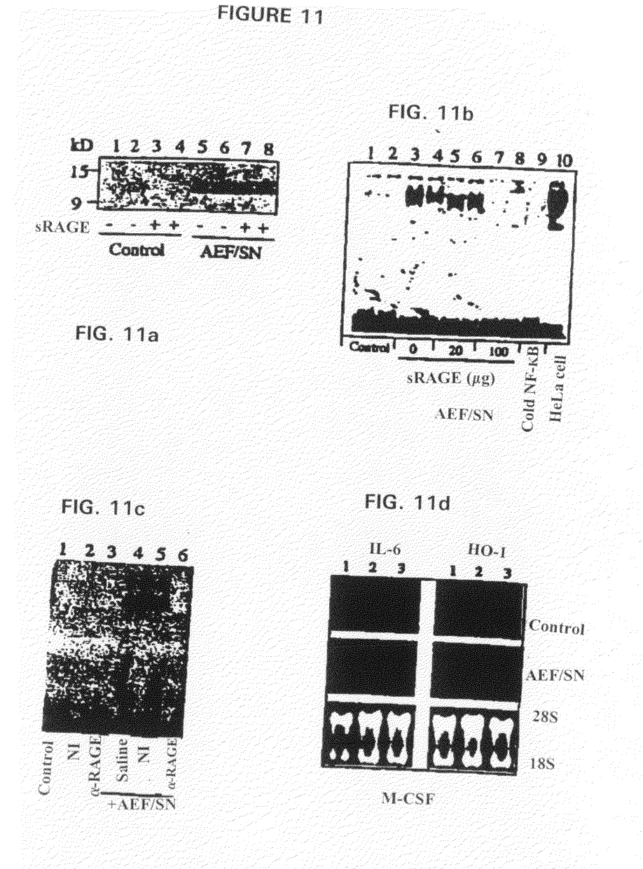

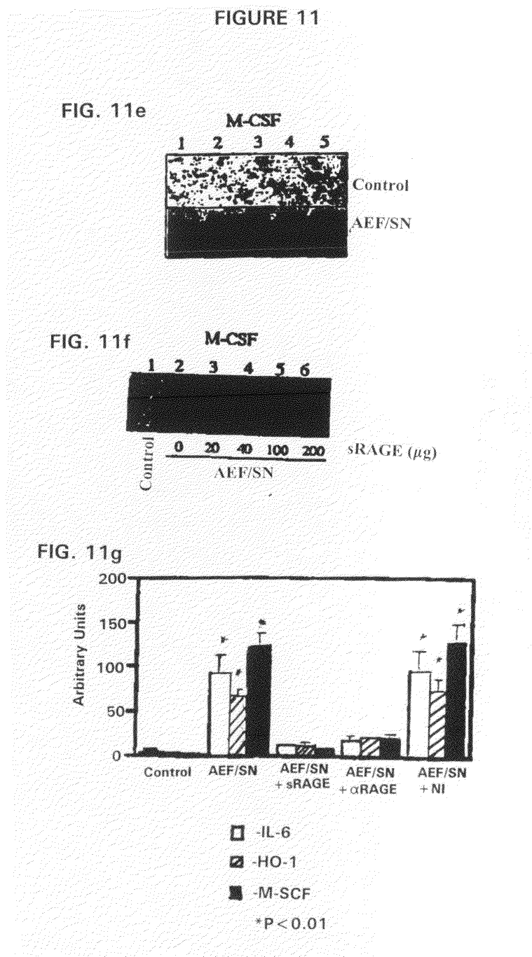

An essential test of our concept concerning RAGE as a receptor for amyloid A was to use a mouse model of systemic reactive amyloidosis, and to assess the effect of RAGE blockade. In this model, we injected C57B1/6 mice with amyloid-enhancing factor (AEF) and silver nitrate (SN) over 5 days^{7,10}. Rapid accumulation of splenic amyloid shows the acute consequences of an environment rich in β-sheet fibrils^{7,10}. Immunoblotting showed almost-undetectable immunoreactive SAA in plasma from control mice (FIG. 11a, lanes 1-4), compared with increased levels in mice receiving AEF/SN (FIG. 11a, lanes 5-8). This was accompanied by evidence cellular perturbation in the spleen as assessed by activation of NF-kB and expression of target genes²³, including IL-6, HO-1 and M-CSF (described below). We used EMSA to study NF-kB activation in mice treated with AEF/SN (FIG. 11b and c). Although nuclear extracts from spleens of control mice showed only a weak or absent gelshift band (FIG. 11b, lanes 1 and 2, and c, lanes 1-3), the intensity of this band increased considerably with treatment with AEF/AN (FIG. 11b, lanes 3 and 4, and c, lanes 4 and 5). This nuclear binding activity was specific for NF-κB, as it was blocked by inclusion of excess unlabeled NF-κB probe (FIG. 11b, lane 59).

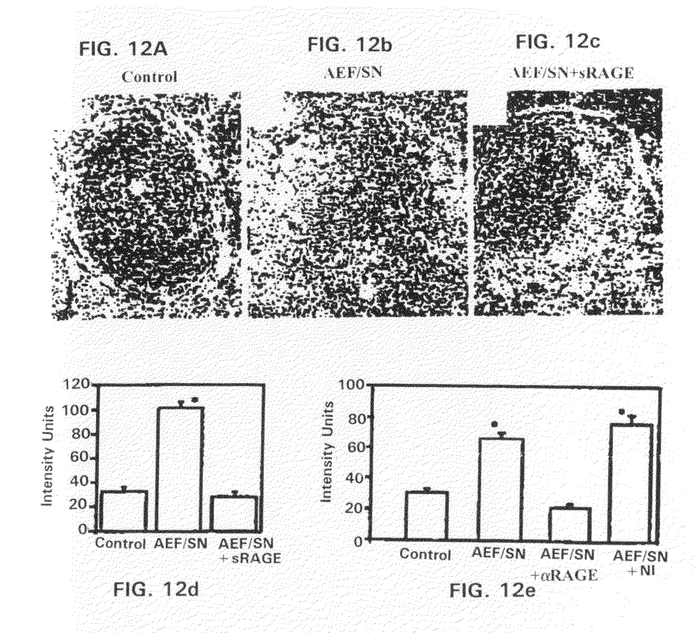

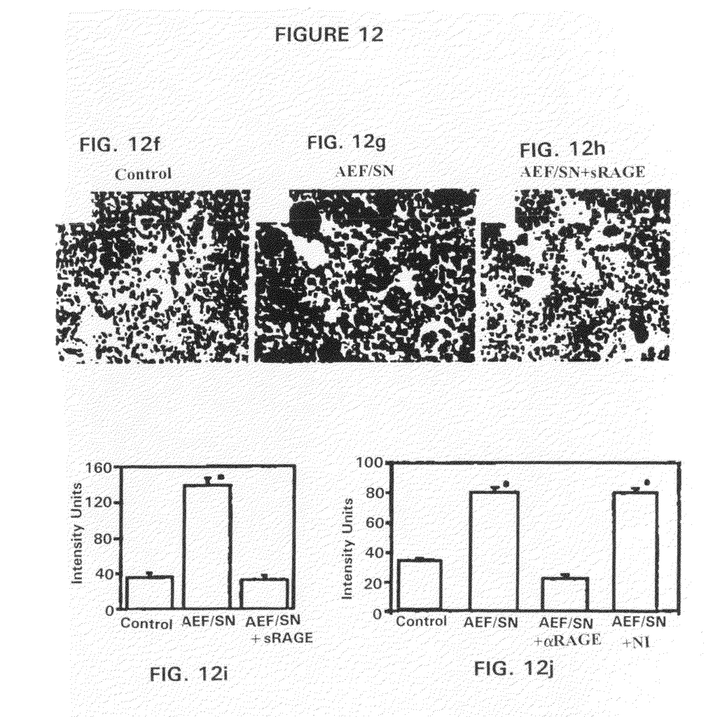

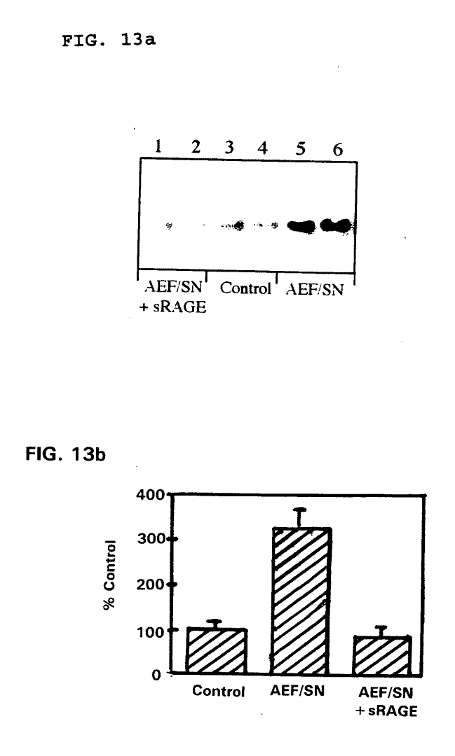

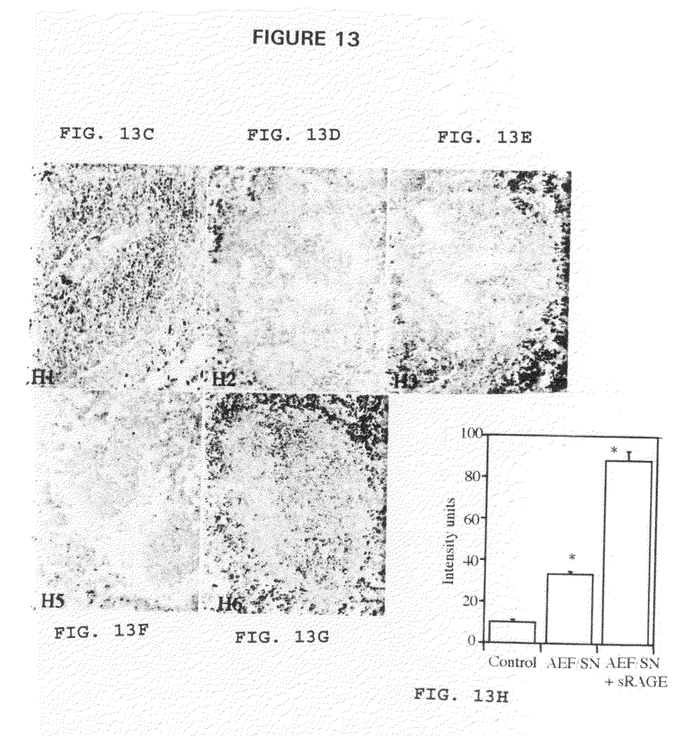

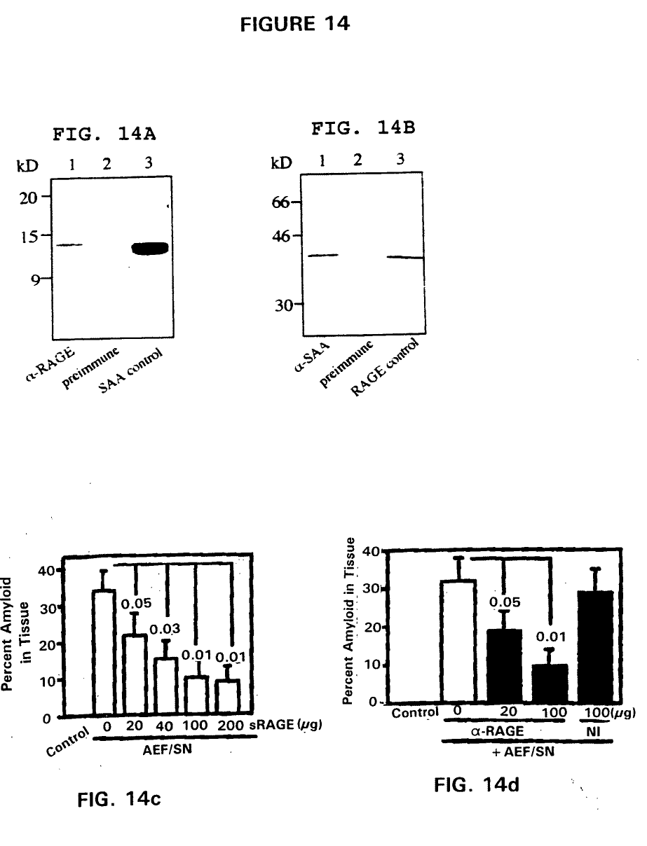

Next, we assessed expression of NF-kB target genes based on our in vitro results with BV-2 cells and SAA1.1, and our evaluation of tissue from a patient with systemic reactive amyloidosis. Total RNA isolated from spleens of control mice showed low levels of IL-6, HO-1 and M-CSF mRNA (FIG. 11 d-g). In contrast, after treatment with AEF/SN, transcripts for each of these genes increased considerably. Consistent with these data, splenic IL-6 antigen was increased in mice treated with AEF/SN, compared with that in samples from untreated control mice (FIGS. 12a and b). Semiquantitative analysis of immunohistochemical images showed an increase in staining intensity of about 200-330% in mice treated with AEF/SN compared with that in control mice (FIGS. 12d and e). Also, there was more staining for M-CSF in splenic mononuclear phagocytes from mice treated with AEF/SN than those from control mice (FIG. 12f and g). Image analysis showed an increase in staining intensity of about 200-320% in mice receiving AEF/SN compared with that in mice receiving no treatment (FIGS. 12i and j). Along with the accumulation of splenic amyloid in mice treated with AEF/SN, compared with that in control mice (FIGS. 13 and 14), these data show a strong association between increased tissue amyloid burden, NF-kB activation and expression of cellular stress markers.

The relevance of RAGE biology to this model of systemic amyloidosis was demonstrated by analysis of RAGE expression in the spleen. Northern blot analysis showed a low level of RAGE transcripts in controls, which increased by about 320% after exposure to AEF/SN (FIGS. 13a and b). RAGE antigen in the spleen, also at low levels in control mice (FIG. 13c), increased after treatment with AEF/SN (FIG. 13d) by about 350% (FIG. 13h). The pattern of deposition of SAA that could be immunostained in the spleens of mice treated with AEF/SN, compared with its near-absence in control mice (FIGS. 13f and g), provided a useful point of reference for localizing of RAGE in the spleen. The distribution of endogenous RAGE in mice treated with AEF/SN overlapped closely that of amyloid A in the spleen (FIGS. 13d and g), consistent with the likelihood that RAGE interaction with amyloid A fibrils occurred in vivo. If this were true, blocking access of amyloid A to RAGE might suppress evidence of cellular perturbation, and, potentially, have an effect on accumulation of fibrils in the tissue as well.

We used two strategies for blocking RAGE: administration of sRAGE (at concentrations that would probably achieve a molar excess of soluble receptor to that of fibrils locally) starting the day before AEF/SN treatment and continuing throughout day 4 of the 5-day experimental period; and treatment with blocking antibody against RAGE F(ab′)₂ (using nonimmune F(ab′)₂ at the same concentration as a control), according to the same protocol. In each case, sRAGE or antibody against RAGE F(ab′)₂ was given once daily intraperitoneally.

Levels of SAA in the plasma remained similarly increased in mice treated with AEF/SN, whether they were given vehicle (mouse serum albumin; FIG. 11a, lanes 5 and 6) or sRAGE (FIG. 11a, lanes 7 and 8). We obtained similar results for plasma SAA in mice given either antibody against RAGE F(ab′)₂ or nonimmune F(ab′)₂ (data not shown). Despite continued high levels of plasma SAA, there was suppression of NF-κB activation in nuclear extracts from mice treated with AEF/SN and sRAGE; the gelshift band in mice treated with AEF/SN was undetectable at the 100-μg dose of sRAGE (FIG. 11b, lanes 7 and 8). Also, in mice treated with AEF/SN receiving 100 g antibody against RAGE F(ab′)₂, there was a prominent decrease in intensity of the gelshift band by EMSA (FIG. 11 c, lane 6), compared with that in mice treated with AEF/SN and receiving saline or nonimmune F(ab′)₂ (FIG. 11c, lanes 4 and 5, respectively). In parallel with decreased activation of NF-κB in mice treated with AEF/SN and infused with sRAGE or antibody against RAGE F(ab′)₂, splenic transcripts for M-CSF antibody (FIGS. 11f and g), HO-1 (FIG. 11g), and IL-6 (FIG. 11g), were substantially decreased in samples from mice given AEF/SN and treated with either of these strategies (sRAGE or antibody against RAGE F(ab′)₂) for blocking cellular RAGE. As expected, given the decrease in IL-6 and M-CSF transcripts in mice treated with AEF/SN and given sRAGE or antibody against RAGE F(ab′)₂, there was a parallel decrease in immunoreactive splenic IL-6 (FIGS. 12c and d, sRAGE, and e, aRAGE F(ab′)₂) and M-CSF antigens (FIGS. 12h and i, sRAGE, and j, aRAGE F(ab′)₂).

Consistent with the possibility that sRAGE, at the doses given, prevented amyloid A fibrils from interacting with cell surface RAGE in mice treated with AEF/SN, immunostaining of splenic tissue from mice treated with AEF/SN plus sRAGE showed an increase in RAGE staining (FIG. 13e), which closely overlapped the AEF/SN-induced expression of endogenous RAGE (FIG. 13d) and deposition of amyloid A (FIG. 13g). The likelihood that the latter increase in RAGE antigen was due to the injected sRAGE rather than enhanced expression of endogenous receptor was strengthened by the suppression of RAGE transcripts in mice treated with AEF/SN and given sRAGE down to levels seen in control mice (not treated with AEF/SN) (FIG. 13a, lanes 1 and 2, and b). These data indicated that RAGE and amyloid A were appropriately juxtaposed to favor their interaction in vivo. Immunoprecipitation of plasma from mice given AEF/SN and treated with sRAGE using anitbody against RAGE IgG, followed by immunoblotting of precipitated material with antibody against SAA IgG, showed two immunoreactive bands (of about 14 and 9 kDa) not seen when preimmune IgG was used in place of antibody against RAGE IgG (FIG. 14a, lanes 1 and 2). In contrast, immunoprecipitation of plasma from mice treated with AEF/SN plus sRAGE with antibody against apolipoprotein SAA (apoSAA), followed by immunoblotting of precipitated material with antibody against RAGE IgG, showed RAGE-immunoreactive material (FIG. 14b, lane 1) that co-migrated with purified sRAGE (FIG. 14b, lane 3). Thus, the SAA-amyloid A-sRAGE complex was present in plasma of mice given AEF/SN and treated with soluble receptor, consistent with a direct interaction of RAGE with the amyloid. The SAA-amyloid A-sRAGE complex was not detected on high-density lipoprotein (HDL) particles (data not shown), indicating that the association was not likely to be through circulating lipoproteins.

The observation that RAGE (both cell surface receptor and infused sRAGE) was likely to interact with amyloid A fibrils indicated that the receptor might directly affect the tissue amyloid burden. There was dose-dependent suppression of splenic amyloid (up to 60%) in sRAGE-treated mice given AEF/SN, compared with that in mice receiving vehicle (mouse serum albumin) alone (FIG. 14c). Although the mechanism through which sRAGE decreased splenic amyloid remains to be determined, it is possible that sRAGE-mediated inhibition of fibril anchoring to the cell surface promotes local clearance of the amyloid. Consistent with the close interaction of RAGE with nascent amyloid was the presence of a more rapidly migrating SAA-immunoreactive band (relative molecular mass, about 9 kDa) in the sRAGE-amyloid A complex (FIG. 14a, lane 1), in addition to the more slowly migrating band corresponding to apparent molecular weight of native/plasma SAA (relative molecular mass, about 14 kDa; FIG. 14a, lanes 1 and 3). Cleave of intact apoSAA1.1 in the tissue, presumably after dissociation of SAA1.1 from HDL, is an integral part of fibrillogenesis²⁴. Furthermore, as administration of antibody against RAGE F(ab′)₂, but not nonimmune F(ab′)₂, also similarly suppressed splenic amyloid A in mice treated with AEF/SN (FIG. 14d), this supports the likelihood that cell surface RAGE is central in the deposition of amyloid A fibrils.

### RAGE Binding of Amylin and Prion-Derived Peptides

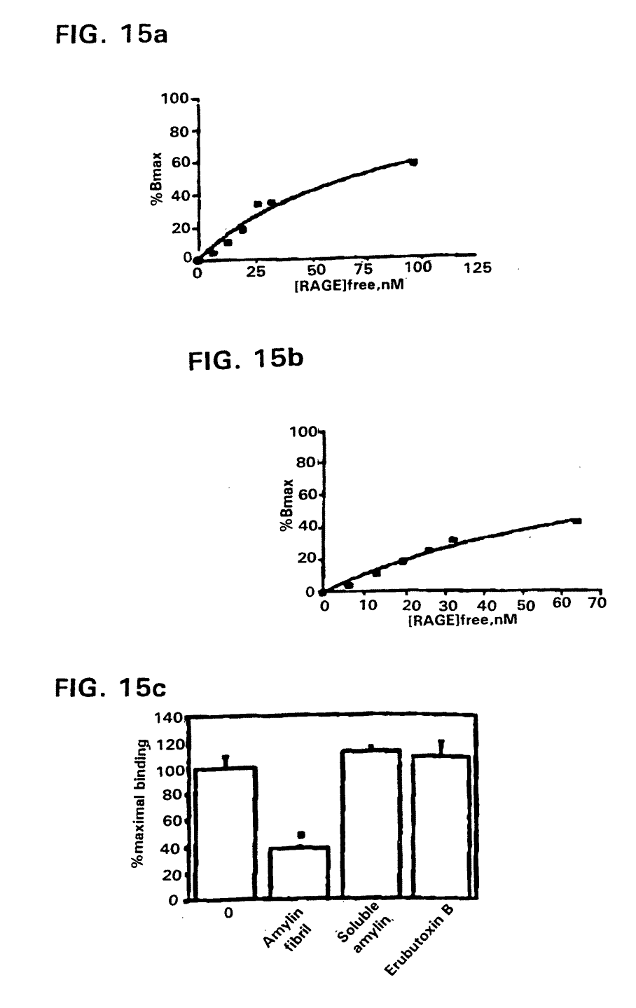

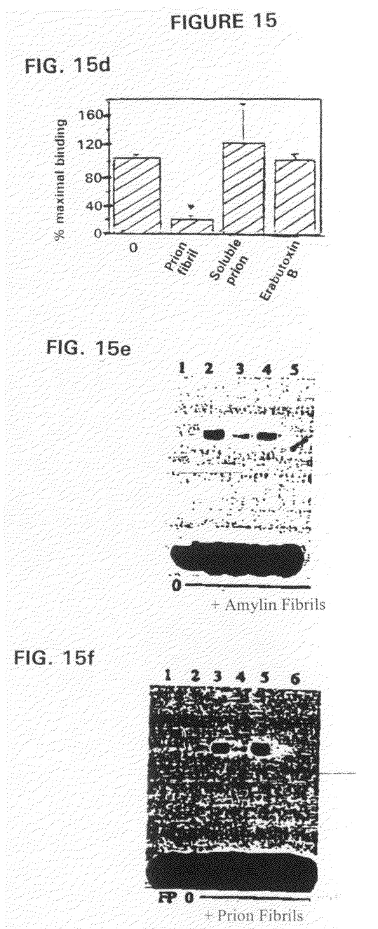

Given the binding of RAGE to amyloid A and the amyloidogenic form of SAA (SAA1.1), the receptor might also interact with other β-sheet fibrils. Preformed fibrils of amylin and prion-derived peptide also bound sRAGE in a dose-dependent manner, with K_{d} values of about 68 and 86 nM, respectively (FIGS. 15a and b). This was similar to the results for the binding of sRAGE to amyloid A and SAA1.1 (FIG. 10c). As these peptides do not show sequence homology, the results indicated that the receptor recognition unit is a structural motif common to amyloid fibrils. Consistent with this, neither amylin nor prion-derived peptide presented to RAGE in random conformation demonstrated inhibition of the binding of ¹²⁵I-sRAGE to the respective fibrillar forms (FIG. 15c and d). It is widely accepted that amyloid fibrils are assembled by interactions between the β-strands of several peptide monomers forming aggregated intermolecular β-sheets, a structure known as cross-conformation 25. To determine whether any protein adopting β-sheet structure would interact with RAGE, we used competitive binding studies with erabutoxin B, a well-known all-i sheet protein that does not form amyloid²⁶; there was no competition (FIG. 15c and d). Similarly, non-cross-β fibrils did not interact with sRAGE; neither collagen nor elastin fibrils interacted with RAGE in the same competitive binding assay (not shown). These data support the concept that RAGE recognizes protein aggregates in the form of β-cross-structured amyloid fibrils.

RAGE also functioned as a signal transduction receptor for amylin and prion-derived peptide fibrils. Incubation of BV-2 cells with fibrils derived from either of these peptides showed activation of NF-κB in nuclear extracts studied by EMSA (FIG. 15e, lanes 1 and 2, and f, lanes 2 and 3). In each case, nuclear translocation of NF-κB could be prevented by addition of antibody against RAGE F(ab′), (FIG. 15e, lane 3, and f, lane 4), but not by nonimmune F(ab′)₂ (FIG. 15e, lane 4, and f, lane 5), to incubation mixtures of fibril preparations and BV-2 cells. Inhibition of the appearance of the gel-shift band by excess unlabeled NF-κB added to nuclear extracts from BV-2 cells exposed to each of the fibrils indicated specificity of the DNA binding activity (FIG. 15e, lane 5, and, f, lane 6).

### Discussion

Amyloidoses share in common deposition of β-sheet fibrillar structures, although the subunits making up the fibrils are diverse. The tissue response to amyloids also shares certain features beyond fibrillogenesis, such as induction of differing degrees of inflammatory reaction, especially involving mononuclear phagocytes. For example, activation of microglial cells by amyloid α-protein, relevant to Alzheimer disease, elicits production of mediators with toxic effects for neurons in vitro²⁷⁻²⁸. We have shown here amyloid-A-induced activation of a mononuclear phagocyte/microglial cell line in vitro and in splenic mononuclear phagocytes in vivo, the latter based on expression of M-CSF. M-CSF is a cytokine particularly pertinent to macrophage function, as it promotes mononuclear phagocyte survival in response to cell stress (for example, in an environment rich in amyloid β-protein)²⁹ and induces cellular activation^{30,31}. Moreover, M-CSF can initiate an autocrine feedback loop; as mononuclear phagocytes express c-fms, the receptor for M-CSF (ref. 32), sustained effects of M-CSF may fundamentally change the course of the host response.

Our study supports the results of clinical observations pertaining to modulation of cellular properties by systemic amyloids. In an analysis of patients with systemic amyloidosis (amyloid A and light-chain amyloid), there was increased expression of TNF-a and M-CSF (ref. 11). Although TNF-α seemed most closely related to the underlying inflammatory process in reactive amyloidosis, M-CSF expression was associated with both amyloid A and light-chain amyloid, and seemed to be linked to ongoing amyloidosis. Evidence of lipid peroxidation products associated with amyloid deposits in systemic amyloidosis supports the view that fibrillogenesis potentially has an effect on cellular properties³³.

The receptor RAGE has properties indicating it could be a common denominator of the cellular response to tissue amyloid in these seemingly diverse disorders. RAGE binds amyloids composed of several types of subunits, including SAA1.1, amylin, prion peptide and amyloid β-protein²¹. Binding requires assembly into β-sheet fibrils (SAA1.1, amylin and prion-derived peptide), though the situation is less clear with amyloid β-protein, for which both fibrillar and monomeric preparations interact with RAGE (because of the rapid transition from monomeric amyloid β-protein in random conformation to β-sheet fibrils in the conditions of the binding assays, the exact form of amyloid β-protein bound to the receptor has not yet been determined). Another property of RAGE consistent with involvement of the receptor in fibrillogenic disorders is related to its induction in chronic diseases such as systemic amyloidosis, atherosclerosis, Alzheimer disease and diabetic complications^{19,21,34,35}. Sustained expression of the receptor in proximity to ligand(s) allows RAGE to exert potentially profound effects on cellular properties. Although RAGE binds several ligands, these interactions seem to be physiologically relevant, as receptor blockade suppresses vascular hyperpermeability in diabetic rats³⁵ and accelerated lesion formation in diabetic, atherosclerosis-prone mice¹⁹. In the latter situations, advanced glycation end-products are likely to represent important RAGE ligands. In our studies of reactive systemic amyloidosis, complexes of sRAGE with amyloid A were immunoprecipitated from plasma. These complexes were not associated with HDL, and included SAA-immunoreactive material with relative molecular masses of about 9 and 14 kDa. As cleavage of SAA is intimately associated with amyloid formation, these data support the possibility of a direct interaction of between RAGE and amyloid A. In addition to possible effects of sRAGE on the clearance of amyloid A, our results demonstrating inhibition of cellular activation and amyloid accumulation in mice treated with antibody against RAGE F(ab′)² (similar to that in mice given sRAGE) emphasize the importance of the binding of amyloid to cellular RAGE in the pathogenesis of systemic amyloidosis.

These results raise the question as to what the physiologic function of RAGE might be. The ligands for RAGE mentioned above, β-sheet fibrils and advanced glycation endproducts (the latter are late-stage adducts formed by nonenzymatic glycoxidation of macromolecules which form at accelerated rates in patients with diabetes)³⁶, cannot be considered endogenous or ‘natural’ ligands. Instead, these are more likely to be ‘accidental’ ligands that interact with the receptor in a sustained manner because of their persistent accumulation in tissues. To begin to address the physiologic functions of RAGE, we have turned to the normal tissue in which receptor expression is greatest, the lung³⁷. Based on an extensive series of studies, we determined that RAGE is a receptor for ligands in the S100/calgranulin and amphoterin families^{13,38}. Each of these groups of polypeptides has properties of inflammatory mediators, among their other activities^{39,40}. Indeed, blockade of RAGE prevents induction of delayed-type hypersensitivity and inflammatory colitis in IL-10-null mice¹³. The latter effect correlated most closely with inhibition of RAGE interaction with S100/calgranulins. Thus, in physiologic conditions RAGE may participate in the orchestration of the inflammatory response. However, in a setting in which a RAGE ligand is present for an extensive time in the tissue, as in amyloidoses, a transient, presumably protective RAGE-dependent inflammatory response may be changed to a chronic destructive inflammatory process. Further studies will be required to fully test the predictions of this hypothesis.

Our work emphasizes the likely dynamic interaction of amyloid A (as well as other amyloids) with the cellular microenvironment, in contrast to a view of amyloid as simply a space-occupying, biologically inert material. Thus, accumulation of amyloid A in tissues may not occur passively; induction of cell stress responses may triggered with activation of NF-κB and expression of target genes. Furthermore, blockade of cell surface RAGE inhibited, at least in large part, accumulation of amyloid and cellular activation. Therefore, assembly of β-sheet fibrils may result in a ‘gain of function’, by allowing fibrillar assemblies to interact with RAGE. The pathophysiological effect of this interaction indicates with RAGE. The pathophysiological effect of this interaction indicates the possibility that RAGE may be a clinically relevant target in amyloidoses to be exploited as a basis of future therapeutic strategies.

### Methods

RAGE-related reagents. Mouse and human sRAGE were expressed using the baculovirus system and purified to homogeneity^{19,38}. Monospecific IgG polyclonal rabbit antibody against human and mouse RAGE, against human or mouse sRAGE, were prepared as described^{19,21,38}. F(ab′)₂ fragments were obtained from IgG, both IgG antibody against RAGE and non-immune rabbit IgG, using a kit from Pierce (Rockford, Ill.), as described¹³. Preparations were tested for endotoxin using the limulus amebocyte assay (Sigma); no endotoxin was detectable at a protein concentration of 2 mg/ml. A vector encoding dominant negative RAGE, which spans the extracelluar and transmembrane domain (but without the cytosolic tail), called pcDMA3-DN-RAGE, was used in cell transfection studies with the lipofectamine method (Life Technologies)^{13,41}. BV-2cells, a transformed mouse microglial line, were grown as described²⁰.

Imaunoblotting and immocytochemistry. Immunoblotting used nonfat dry milk and either rabbit IgG antibody against human/mouse RAGE (3.3 μg/ml) or against SAA (1 μg/ml; this antibody cross-reacts with amyloid A fibrils isolated from mouse splenic tissue, and recognizes both SAA2.1 and SAA 1.1)⁶. Sites of primary antibody binding were identified with peroxidase-conjugated antibody against rabbit IgG (1:2,000 dilution, Sigma) by the enhanced chemiluminescence method (ECI; Amersham) and autoradiograms were analyzed by laser densitometry. Immunohistological analysis of mouse tissues from the systemic amyloid mode used paraformaldehyde-fixed, paraffin-embedded sections (5-6 μm ir thickness) with 50 μg/ml rabbit IgG antibody against mouse IL-6 (provided by G. Fuller, University of Alabama, Birmingham), 4 μg/ml goat IgG antibody against mouse M-CSF (Santa Cruz Biotechnology, Santa Cruz, Calif.), 1 μg/ml rabbit IgG antibody against SAA and 50 μg/ml IgG antibody against RAGE, and the Biotin-ExtrAvidin Alkaline Phosphatase Kit (Sigma). Quantification of microscopic images was accomplished with the Universal Imaging System (West Chester, Pa.). Splenic tissue sections, formalin-fixed and paraffin-embedded as described above, were analyzed from a patient without evidence of amyloid (69-year-old male who died of cardiovascular disease) and a patient with systemic amyloidosis due to chronic granulomatous pulmonary disease from Histoplasma Capsulatum (71-year-old male with extensive amyloid deposition, including the liver, spleen, kidneys and so on). Immunostaining was done as described for mouse tissues above, using 30 μg/ml rabbit IgG antibody against human RAGE, 10 μg/ml mouse IgG monoclonal antibody against CD14, 20 μg/ml rabbit IgG antibody against human IL-6 and 20 μg/ml IgG antibody against human M-CSF (all from Santa Cruz Biotechnology, Santa Cruz, Calif.) Double staining (for CD14 and RAGE) was accomplished by first incubating sections with mouse IgG antibody against CD14 followed by detection with biotin-conjugated goat antibody against mouse IgG and ExtrAvidin-conjugated alkaline phosphatase (with Fast Red as the substrate) (Sigma). After visualization of CD14 antigen, sections were decolorized with 95% ethanol, washed with PBS and incubated in 3% hydrogen peroxide/methanol for 10 min. Samples were then washed in PBS again, and incubated with IgG antibody against RAGE (as described above; primary antibody) using peroxidase-conjugated goat antibody against rabbit IgG (secondary antibody) and 3-amino-9-ethyl carbazole (AEC; Sigma) as the detection system.

Preparation of fibrils. Prion peptide (residues 109-141; Biosynthesis, Louisville, Tex.) and human amylin (MRL, Herndon, Va.) fibrils were made by dissolving peptide solutions in PBS at a concentration of 2.0 mg/ml for amylin and 2.5 mg/ml for prion-derived peptide, and incubating these for 4 d at 37° C. Fibril formation was assessed by electron microscopy and secondary structure was determined by circular dichroism spectroscopy. The peptide/protein secondary structure in solution was: prion-derived peptide, 75% random; amylin, 80% random; erabutoxin B (Sigma), 90% β-sheet. There was no evidence of fibrillogenesis in preparations of random-conformation prion-derived peptide and amylin, or erabutoxin B, based on electron microscopy. Pellets were made from fibril preparations by centrifugation, and were resuspended in PBS, pH 7.4, subjected to five strokes of the sonicator, separated into aliquots and frozen at −20° C. After being thawed, preparations were used immediately. The concentration of fibrillar preparations is derived from that of the monomer initially added to the mixture to make fibrils. Aβ₁₋₄₀ was obtained from QCB (Biosouce international, Hopkinton, Massachusettes). Mouse SAA2.1, SAA1.1, SAA2.218, AI and AII were prepared from HDL isolated from plasma of C57B1/6 and CE/J mice subject to acute-phase stimulation by intraperitoneal injection of lipopolysaccharide (Escherichio Coli 0111:B4; Difco Laboratories, Detroit, Mich.). HDL was isolated from plasma by potassium bromide density centrifugation^{14,17}, and de-lipidated HDL was separated on a Sephacryl S200 column equilibrated with 8M urea and 10 mM Tris-HCL, pH 8.2. Peak SAA samples were fractionated on DEAE-Sephacel in the same buffer, and were eluted with a linear gradient of sodium chloride to 150 mM. Fractions were analyzed by SDS-PAGE and immunoblotting and isoelectric focusing to verify SAA isoform. Amyloid A fibrils were purified from spleens of mice treated with AEFISN as described⁴².

RAGE-fibril binding assays. Binding assays were done in a purified system by incubating protein or peptide preparations for 20 h at 4° C. in carbonate/bicarbonate buffer in micotiter wells (Nunc Maxisorp, VWR, West Chester, Pa.) to allow adsorption, blocking them for 2 h at 37° C. with PBS containing albumin (10 mg/ml), and then incubating them for 2 h at 37° C. with the addition of ¹²⁵I-sRAGE (either alone or in the presence of an excess of unlabeled sRAGE) in minimal essential medium with 10 mM HEPES, pH 7.4, and 1 mg/ml fatty-acid-free bovine serum albumin. Where indicated, soluble amylin or prion-derived peptide in random conformation, erabutoxin B (Sigma) or amylin or prion-peptide-derived fibrils were added as unlabeled competitors in the binding assay. After the incubation period, the reaction mixture was removed, and wells were washed four times over 30 s with ice-cold PBS containing 0.05% Tween-20. Bound ¹²⁵I-sRAGE was eluted for 5 min at 37° C. with 1% Nonidet-P40, and bound ligand was quantified by measuring radioactivity. sRAGE was radiolabeled by the Iodobead method (Pierce, Rockford, Ill.) 38, and binding data were analyzed as described⁴³.

Experiments with cultured BV-2 cells. Cultured BV-2 cells were incubated at 37° C. with SAA1.1, amylin or prion-peptide-derived fibrils (for the last, the concentration was that of the monomer making up the fibril). Then, nuclear extracts were prepared and an EMSA was done with ³²P-labeled consensus probe for NF-κB as described²¹. In other experiments, total RNA was collected from BV-2 cells and northern blot analysis was done using ³²P-labeled mouse cDNA probes (HO-1, IL-6 and M-CSF). For 11a, lanes 6-9, BV-2 cells were transfected with pcDNA3-DN-RAGE or pcDNA3 alone. Cultures were incubated for 5 h at 37° C. with a mixture of 7 μl lipofectamine per 60-mm dish and 2 μg DNA mixture in serum-free Opti-MEM (Life Technologies). Then, serum-containing medium was added to a final serum concentration of 10% for 48 h of incubation, and cultures were exposed to fibrils in serum-free DMEM. Expression of the transfected gene was confirmed by immunoblotting (dominant negative RAGE moves more rapidly during SDS-PAGE than does full-length RAGE).

Mouse model of systemic amyloidosis. C57B1/6/J mice 2-4 months of age were injected with 100 μg AEF and 0.5 ml of a 2% solution of 5N for 5 d to induce amyloid deposition, and were killed on day 5 (refs. 6,7,10). For these experiments, there were five mice per group. Mice were treated with either recombinant mouse sRAGE, antibody against RAGE F(ab′)₂ nonimmune F(ab′)₂ saline or mouse serum albumin by daily intraperitoneal injection starting at day −1 (day 0, start of AEF/SN treatment) and continuing to day 4. For analysis of amyloid deposition, mice were perfused with ice-cold saline followed by 4% buffed paraformaldehyde, and spleens were ‘postfixed’ for 24 h in 4% paraformaldehyde⁶. Tissues were embedded in paraffin and proceed as described above.

Congo red staining was done as described⁷, and amyloid burden was quantified using image analysis on immunostained (antibody against SAA IgG) and Congo-red-stained (polarized light) sections^{6,10}. The amyloid burden in tissue sections was compared with standards for quantification. For northern blot analysis, the spleen was cut into small pieces, immersed in Trizol (Life Technologies) and homogenized, and total RNA was extracted and separated by 0.8% agarose gel electrophoresis. RNA was transferred to Duralon-UV membranes (Stratagene, La Jolla, Calif.), and membranes were then hybridized with ³²P-labeled cDNA probes for mouse RAGE, HO-1, IL-6 and M-CSF.

### REFERENCES

Akama, K., Albanese, C., Pestell, R., and Van Eldik, L. (1998). Aβ stimulates nitric oxide production in astrocytes through an NF-kB-dependent mechanism. Proc Natl Acad Sci (U.S.A.)95, 5795-5800.
Aleshkov, S., Abraham, C., and Zannis, V. (1997). Interaction of nascent ApoE2, ApoE3, and ApoE4 isoforms expressed in mammalian cells with amyloid peptide beta (1-40): relevance to Alzheimer's disease. Biochemistry 36, 10571-10580.
Behl, C., Davis, J., Lesley, R., and Schubert, D. (1994). Hydrogen peroxide mediates Aβ toxicity. Cell 77, 817-827.
Blacker, D., Wilcox, M., Laird, N., Rodes, L., Horvath, S., Go, R., Perry, R., Watson, B., Bassett, S., McInnis, M., Albert, M., Hyman, B., and Tanzi, R. (1998). Alpha-2-macroglobulin is genetically associated with AD. Nature Genet. 19, 357-360.
Boland, K., Behrens, M., Choi, D., Manias, K., and Perlmutter, D. (1996). The SEC receptor recognizes soluble, nontoxic Aβ but not aggregated, cytotoxic Aβ. J. Biol. Chem. 271, 18032-18044.
Combs, C., Johnson, D., Cannady, S., Lehman, T., and Landreth, G. (1999). Identification of microglial signal transduction pathways mediating neurotoxic response to amyloidogenic fragments of Aβ and prion proteins. J. Neurosci. 19, 928-939.
deBeer, M., deBeer, F., McCubin, W., Kay, C., and Kindy, M. (1993). Structural prerequisites for serum amyloid A fibril formation. J. Biol. Chem. 268, 20606-20612.
DiGabriele, A., Lax, I., Chen, D., Svahn, C., Jaye, M., Schlessinger, J., and Hendrickson, W. (1998). Structure of a heparin-linked biologically active dimer of FGF. Nature 393, 812-817.
Forloni, G., Tagliavini F, Bugiani, O., and Salmon, M. (1996) Amyloid in AD and prion-related encephalopathies: studies with synthetic peptides. Prog. Neurobiol. 49, 287-315.
Funato, H., Yoshimura, M., Kusui, K., Tamaoka, A., Ishikawa, K., Ohkoshi, N., Namekata, K., Okeda, R., and Ihara, Y. (1998). Quantitation of Aβ in the cortex during aging and in Alzheimer's disease. Am. J. Pathol. 152, 1633-1640.
Ghiso, J., Wisniewski, T., and Frangione, B. (1994). Unifying features of systemic and cerebral amyloidosis. Molec. Neurobiol. 8, 49-63.
Gillardon, F., Skutella, T., Ohlmann, E., Holsboer, F., Zimmermann, M., and Behl, C. (1996). Activation of c-Fos contributes to Aβ-induced neurotoxicity. Brain Res. 706, 169-172.
Giulian, D., Haverkamp, L., Yu, J., Karshin, W., Tom, D., Li, J., Kazanskaia, A., Kirkpatrick, J., and Roher, A. (1998). The HHQK domain of Aβprovides a structural basis for the immunopathology of Alzheimer's disease. J. Biol. Chem. 273, 29719-29726.
Gomez, Y., Kaplan, G., Racaniello, V., and Hogle, J. (1993). Characterization of poliovirus conformational alteration mediated by soluble cell receptors. Virology 197, 501-505.
Guo, Q., Fu, W., Luo, H., Sells, S., Geddes, J., Bondada, V., Rangnekar, V., and Mattson, M. (1998). Par-4 is a mediator of neuronal degeneration associated with the pathogenesis of Alzheimer disease. Nature Med. 4, 957-962.
Hardy, J. (1997). Amyloid, the presenilins and Alzheimer's disease. Trends Neurosci. 556, 28570-26998.
Hensley, K., Carney, J., Mattson, M., Aksenova, M., Harris, M., Wu, J., Floyd, R., and Butterfield, D. (1994). A model for Aβ aggregation and neurotoxicity based on free radical generation by the peptide: relevance to AD. Proc. Natl. Acad. Sci. (U.S.A.) 91, 3270-3274.
Hofmann, M., Drury, S., Caifeng, F., Qu, W., Lu, Y., Avila, C., Kambhan, N., Slattery, T., McClary, J., Nagashima, M., Morser, J., Stern, D., and Schmidt, A-M. (1999). RAGE mediates a novel proinflammatory axis: a central cell surface receptor for S100/calgranulin polypeptides. Cell, in press.
Hori, O., Brett, J., Nagashima, M., Nitecki, D., Morser, J., Stern, D. M., and Schmidt, A. M. (1995). RAGE is a cellular binding site for amphoterin. J. Biol. Chem. 270, 25752-25761.
Inagaki, F., Miyazawa, T., Hori, H., and Tamiya, N. (1978). Conformation of erabutoxins a and b in aqueous solution as studied by NMR & CD. Eur. J. Biochem. 89, 433-439.
Kang, D., Saitoh, T., Chen, X., Xia, Y., Masliah, E., Hansen, L., Thomas, R., Thal, L., and Katzman, R. (1997). Genetic assocation of LRP with late-onset AD. Neurology 49, 56-61.
Kelly, J. (1996). Alternative conformations of amyloidogenic proteins govern their behavior. Curr. Opin. Struct. Biol. 6, 11-17.
Khoury, J. E., Hickman, S. E., Thomas, C. A., Cao, L., Silverstein, S. C., and Loike, J. D. (1996). Scavenger receptor adhesion of microglia to Aβ fibrils. Nature (Lond.) 382, 716-719.
Kimball, M., Sato, A., Richardson, J., Rosen, L., and Low, B. (1979). Molecular conformation of erabutoxin b: atomic coordinates at 2.5 Å resolution. Biochem. Biophys. Res. Comm. 88, 950-956.
Kindy, M., King, A., Perry, G., deBeer, M., and deBeer, F. (1995). Association of apolipoprotein E with murine amyloid A protein amyloid. Lab. Invest. 73, 469-475.
Kindy, M. and Rader, D. (1998). Reduction in amyloid A amyloid formation in apolipoprotein-E-deficient mice. Am. J. Pathol. 152, 1387-1395.
Kirschner, D., Abraham, C., and Selkoe, D. (1986). X-ray diffraction from intraneuronal PHF and extraneuronal amyloid fibrils in AD indicates cross-§ conformation. Proc Natl Acad Sci (USA) 83, 503-507.
Kisilevsky, R., Lemieux, J., Fraser, P., Kong, X., Hultin, P., and Szarek, W. (1995). Arresting amyloidosis in vivo using small molecule anionic sulphonates or sulphates: implications for Alzheimer's disease. Nature Med. 1, 143-148.
Klotz, I. and Hunston, D. (1984). Mathematical models for ligand-receptor binding. J. Biol. Chem. 258, 11442-11445.
Kosik, K. (1994). Alzheimer's disease sphinx: a riddle with plaques and tangles. J. Cell Biol. 127, 1501-1504.
LaDu, M., Lukens, J., Reardon, C., and Getz, G. (1997). Association of human, rat and rabbit apoE with Aβ. J. Neurosci. 49, 9-18.
Lambert, M., Barlow, A., Chrom, B., Edwards, C., Freed, R., Liosatos, M., Morgan, T., Rozovsky, I., Trommer, B., Viola, K., Wals, B., Zhang, C., Finch, C., Krafft, G., and Klein, W. (1998). Diffusible, nonfibrillary ligands derived from Aβ1-42 are potent central nervous system neurotoxins. Proc Natl Acad Sci (USA) 95, 6448-6453.
Lander, H., Tauras, J., Ogiste, J., Moss, R., and Schmidt, A. M. (1997). Activation of RAGE triggers a MAP kinase pathway regulated by oxidant stress. J. Biol. Chem. 272, 17810-17814.
Lansbury, P. T. (1999) Evolution of amyloid: what normal protein folding may tell us about fibrillogenesis and disease Proc Natl Acad Sci (USA) 96, 3342-3344.
Lendon, C., Talbot, C., Craddock, N., Han, S., Wragg, M., Morris, J., and Goate, A. (1997). Genetic association studies between dementia of the Alzheimer's type and three receptors for apoE in a Caucasian population. Neurosci. Lett. 222, 187-190.
Levin, M., Franklin, E., Frangione, B., and Pras, M. (1972). The amino acid sequence of a major nonimmunoglobulin component of some amyloid fibrils. J. Clin. Invest. 51, 2773-2776.
LeVine, H. (1993). Thioflavine T interaction with synthetic Alzheimer's beta-amyloid peptides: detection of amyloid aggregation in solution. Protein Science 2, 404-410.
Li, J. and Schmidt, A-M. (1997). Characterization and functional analysis of the promoter of RAGE. J. Biol. Chem. 272, 16498-16506.
Liao, A., Nitsch, R., Greenberg, S., Finckh, U., Blacker, D., Albert, M., Rebeck, G., Gomez-Isla, T., Clatworthy, A., Binetti, G., Hock, C., Mueller-Thomsen, T., Mann, U., Zuchowski, K., Beisiegel, U., Staehelin, H., Growdon, J., Tanzi, R., and Hyman, B. (1998). Genetic association of an a₂-macroglobulin (Val1000Ile) polymorphism and Alzheimer's disease. Human Molec. Gen. 12, 1953-1956.
Mackic, J., Stins, M., McComb, J., Calero, M., Ghiso, J., Kin, K., Yan, S-D., Stern, D., Schmidt, A-M., Frangione, B., and Zlokovic, B. (1998). Human Blood-Brain Barrier Receptors for Alzheimer's amyloid beta peptide 1-40. J. Clin. Invest. 102, 734-743.
Mark, R., Blanc, E., and Mattson, M. (1996). Amyloid beta-peptide and oxidative cellular injury in Alzheimer's disease. Mol. Neurobiol. 12, 915-924.
Marshak, D., Pesce, S., Stanley, L., and Griffin, W. (1992). Increased S100 beta neurotrophic activity in Alzheimer's disease temporal lobe. Neurobiol. Aging 13, 1-7.
Matter, M., Zhang, Z., Nordstadt, C., and Ruoslahti, E. (1998). The a₅β₁ integrin receptor binds the AD Aβ protein and mediates its internalization. Keystone Symp. Proc. X5, 101.(Abstract)
Mattson, M. (1995). Free radicals and disruption of neuronal ion homeostasis in Alzheimer's disease: a role for amyloid beta-peptide? Neurobiol. Aging 16, 679-682.
Mattson, M. and Goodman, Y. (1995). Different amyloidogenic peptides share a similar mechanism of neurotoxicity involving reactive oxygen species and calcium. Brain Res. 676, 219-224.
Miyata, T., Oda, O., Inagi, R., Iida, Y., Araki, N., Yamada, N., Horiuchi, S., Taniguchi, N., Maeda, K., and Kinoshita, T. (1993). β₂-microglobulin modified with AGEs is a major component of hemodialysis-associated amyloidosis. J. Clin. Invest. 92, 1243-1252.
Miyata, T., Hori, O., Zhang, J., Yan, S. D., Ferran, L., Iida, Y., and Schmidt, A. M. (1996). RAGE mediates the interaction of AGE-β₂-microglobulin with human monocytes via an oxidant-sensitive pathway. J. Clin. Invest. 98, 1088-1094.
Nakai, M., Hojo, K., Taniguchi, T., Terashima, A., Kawamata, T., Hashimoto, T., Maeda, K., and Tanaka, C. (1998). PKC and tyrosine kinase invovlement in Aβ(25-35)-induced chemotaxis of microglia. Neuroreport 9, 3467-3470.
Narita, M., Holtzman, D., Schwartz, A., and Bu, G. (1997). a₂-macroglobulin complexes with and mediates the endocytosis of Aβ via cell surface LRP. J. Neurochem. 69, 1904-1911.
Paresce, D. M., Ghosh, R. N., and Maxfield, F. R. (1996). Microglial cells internalize aggregates of the Alzheimer's disease Aβ via a scavenger receptor. Neuron 17, 553-565.
Park, L., Raman, K., Lee, K., Lu, Y., Ferran, L., Chow, W., Stern, D., and Schmidt, A-M. (1998) Suppression of diabetic atherosclerosis by sRAGE. Nat. Med. 4, 1025-1031.
Pike, C., Burdick, D., Walencewicz, A., Glabe, C., and Cotman, C. (1993). Neurodegeneration induced by Aβ in vitro: the role of peptide assembly state. J. Neurosci. 13, 1676-1687.
Pollard, H., Arispe, N., and Rojas, E. (1995). Ion channel hypothesis for Alzheimer amyloid peptide neurotoxicity. Cell Mol. Neurobiol. 15, 513-526.
Prelli, F., Pras, M., and Frangione, B. (1987). Degradation and deposition of amyloid AA fibrils are tissue specific. Biochemistry 26, 8251-8256.
Prusiner, S. B., Scott, M., DeArmond, S., and Cohen, F. (1998). Prion protein biology. Cell 93, 337-348.
Roher, A., Chaney, M., Kuo, Y., Webster, S., Stine, W., Haverkamp, L., Woods, A., Cotter, R., Tuohy, J., Krafft, G., Bonnell, B., and Emmerling, M. (1996). Morphology and toxicity of Aβ(1-42) dimer dervied from neuritic and vascular amyloid deposits of Alzheimer's disease. J. Biol. Chem. 271, 20631-20635.
Roses, A. (1998). Apolipoprotein E and Alzheimer's disease. The tip of the susceptibility iceberg. Ann. New York Acad. Sci. 855, 738-743.
Schmidt, A-M., Yan, S-D., Wautier, J-L., and Stern, D. M. (1999). Activation of RAGE: a mechanism for chronic dysfunction in diabetic vasculopathy and atherosclerosis. Circ. Res. 84, 489-497.
Selkoe, D. (1999). Cell biology of the amyloid beta-protein 25 precursor and the mechanism of Alzheimer's disease. Ann. Rev. Cell Biol. 10, 373-403.
Serpell, L., Sunde, M., and Blake, C. (1997). The molecular basis of amyloidosis. Cell Mol. Life Sci. 53, 871-887.
Sheng, J., Mrak, R., Rovnaghi, C., Kozlowska, E., Van Eldik, L., and Griffin, W. (1996). Human brain S100β and S100β mRNA expression increases with age: pathogenic implications for Alzheimer's disease. Neurobiol. Aging 17, 359-363.
Shiroo, M., Kawahara, E., Nakanishi, I., and Migita, S. (1998). Specific deposition of serum amyloid A protein 2 in the mouse. Scand. J. Immunol. 332, 721-728.
Sipe, J., Carreras, I., Gonnerman, W., Cathcart, E., deBeer, M., and deBeer, F. (1993). Characterization of the inbred CE/J mouse strain as amyloid resistant. Am. J. Pathol. 143, 1480-1485.
Sipe, J. D. (1992). Amyloidosis. Ann. Rev. Biochem. 61, 947-975.
Smith, M., Kutty, R., Richey, P., Yan, S.-D., Stern, D. M., Chader, G., Wiggert, B., Petersen, R., and Perry, G. (1994). Heme oxygenase-1 is associated with the neurofibrillary pathology of Alzheimer's disase. Am. J. Pathol. 145, 42-47.
Soto, C., Castano, E., Prelli, F., Kumar, R., and Baumann, M. (1995). Apolipoprotein E increases the fibrillogenic potential of synthetic peptides derived from Alzheimer's gelsolin and amyloid A amyloids. FEBS Lett. 371, 110-114.
Soto, C. and Castano, E. (1996). The conformation of Aβ determines the rate of amyloid formation and its resistance to proteolysis. Biochem. J. 314, 701-707.
Soto, C. (1999). Alzheimer's and prion disease as disorders of protein conformation: implications for the design of new therapeutic strategies. J. Mol. Med. (in press)
Stanley, E., Berg, K., Einstein, D., Lee, P., Pixley, F., Wang, Y., and Yeung, Y. (1997). Biology and action of colony stimulating factor-1. Mol. Reprod. Dev. 46, 4-10.
Strachen, A., deBeer, F., van der Westhuyzen, D., and Coetzee, G. (1988). Identification of three isoform patterns of human serum amyloid A protein. Biochem. J. 250, 203-207.
Strauss, S., Bauer, J., Ganter, U., Jonas, U., Berger, M., and Volk, B. (1992). Detection of IL-6 and a₂-macroglobulin immunoreactivity in cortex and hippocampus of AD patients. Lab. Invest. 66, 223-230.
Terry, R., Masliah, E., Salmon, D., Butters, N., DeTeresa, R., Hill, R., Hansen, L., and Katzman, R. (1991). Physical basis of cognitive alterations in Alzheimer disease: synapse loss is the major correlate of cognitive impairment. Ann. Neurol. 30, 572-580.
Watson, D., Lander, A., and Selkoe, D. (1997). Heparin-binding properties of the amyloidogenic peptides Aβ and amylin. J. Biol. Chem. 272, 31617-31624.
Wood, S., Wetzel, R., Martin, J., and Hurle, M. (1995). Prolines and amyloidogenicity in fragments of the Alzheimer's peptide beta/A4. Biochemistry 34, 724-730.
Yaar, M., Zhai, S., Pilch, P., Doyle, S., Eisenhauer, P., Fine, R., and Gilchrest, B. (1997). Binding of Aβ to the p75 neurotrophin receptor induces apoptosis. J. Clin. Invest. 100, 2333-2340.
Yan, S.-D., Chen, X., Chen, M., Zhu, H., Roher, A., Slattery, T., Zhao, L., Nagashima, M., Morser, J., Migheli, A., Nawroth, P., Stern, D. M., and Schmidt, A.-M. (1996). RAGE and amyloid-§ peptide neurotoxicity in Alzheimer's disease. Nature 382, 685-691.
Yan, S.-D., Zhu, H., Fu, J., Yan, S.-F., Roher, A., Tourtellotte, W. W., Rajavashisth, T., Chen, X., Godman, G. C., Stern, D., and Schmidt, A. M. (1997). Aβ-RAGE interaction elicits neuronal expression of M-CSF. Proc. Natl. Acad. Sci. (USA) 94, 5296-5301.
Yankner, B. (1996). Mechanisms of neuronal degeneration in Alzheimer's disease. Neuron 16, 921-932.
Zheng, H., Jiang, M., Trumbauer, M., Sirinathsinghji, D., Hopkins, R. Smith, D., Heavens, R., Dawson, G., Boyce, S., Conner, M., Stevens, K., Slunt, H., Sisodia, S., Chen, H., and Van der Ploeg, L. (1995). βAPP-deficient mice show reactive gliosis and decreased locomotor activity. Cell 81, 525-531.

### REFERENCES FOR SECOND SERIES OF EXPERIMENTS

1. Slpe, J. Amyloidosis. Annu. Rev. Biochem. 61, 947-975, 1992
2. Falk, R., Comenzo, R. & Skinner, M. The systemic amyloidoses. N. Engl. J. Med 337, 898-908, 1997
3. Gillmore, J., Hawkins, P. & Pepys, M. Amyloidosis: a review of recent diagnostic and therapeutic developments. Br. J. Haematol. 99, 245-256 (1997).
4. Hoshii, Y. et al. Amyloid A protein amyloidosis induced in ApoE-deficient mice. Am J. Pathol. 151, 911-917 (1997).
5. Wisniewski, T. & Frangione, B. ApoE: a pathological chaperone protein in with cerebral and systemic amyloid. Neurosci. Lett. 135:235-238 (1992).
6. Kindy, M & Rader, D. Reduction in amyloid A amyloid formation in apolipoprotein E-deficient mice. Am. J. Pathol. 152, 1387-1395 (1998).
7. Kindy, M., King, A., Perry, G., deBeer, M. & deBeer, F. Association of apolipoprotein E with murine amyloid A protein amyloid. Lab. Invest. 73, 469-475 (1995).
8. Hawkins, P., Tennent, G., Woo, P. & Pepys, M. Studies in vivo and in vitro of serum amyloid P component in normals and in a patient with AA amyloidosis. Clin. Exp. Immunol. 84, 308-316 (1991).
9. Botto, M. et al. Amyloid deposition is delayed in mice with targeted deletion of the serum amyloid P component gene. Nature Med. 3, 855-859 (1997).
10. Kisilevsky, R. et al. Arresting amyloidosis in vivo using small molecule anionic sulphonates or sulphates: implications for Alzheimer's disease. Nature Med. 1, 143-148 (1995).
11. Rysava, R., Merta, M., Tesar, V., Kirsa, M. & Zilma, T. Can Serum amyloid A or macrophage colony stimulating factor serve as a marker of amyloid formation? Biochem. Mol. Biol. Int. 47, 845-850 (1999).
12. Schmidt, A-M., Yan, S-D., Wautier, J-L & Stern, D. Activation of RAGE: a mechanism for chronic dysfunction in diabetic vasculopathy and atherosclerosis. Circ. Res. 84, 489-497 (1999).
13. Hofmann, M. et al. RAGE mediates a novel proinflammatory axis: the cell surface receptor for S100/calgranulin polypeptides Cell 97, 889-901 (1999).
14. Yan, S-D., Roher, A., Schmidt, A-M. & Stern D. Cellular cofactors for amyloid betapeptide induced by cell stress: moving from cell culture to in vivo. Am. J. Pathol. 155, 1403-1411 (1999).
15. Shirmo, M., Kawahara, E., Nakanishi, I. & Migita, S. Specific deposition of serum amyloid A protein 2 in the mouse. Scand. J. Immunol. 332, 721-728 (1998).
16. Strachen, A., deBeer, F., van der Westhuyzen, D. & Coetzee, G. Identification of three isoform patterns of human serum amyloid A protein. Biochem. J. 250, 203-207 (1988).
17. DeBeer, M., deBeer, F., McCubin, W., Kay, C. & Kindy, M. Structural prerequisites for serum amyloid a fibril formation. J. Biol. Chem. 268, 20606-20612 (1993).
18. Sipe, J. et al. Characterization of the inbred CE/J mouse strain as amyloid resistant. Am. J. Pathol. 143, 1480-1485 (1993).
19. Park, L. et al. Suppression of accelerated diabetic atherosclerosis by sRAGE. Nature Med. 4, 1025-1031 (1998).
20. Bocchini, V. et al. An immortalized cell line expresses properties of activated microglial cells J. Neurosci. Res. 31, 616-621 (1992).
21. Yan, S-D. Et al. RAGE and amyloid-beta peptide neurotoxicity in Alzheimer's disease. Nature 382, 685-691 (1996).
22. Sulfredini, A., Fantuzzi, G., Badolato, R., Oppenheim, H. & O'Grady, N. New insights into the biology of the acute phase response. J. Clin. Immunol. 19, 203-214 (1999).
23. Collins, T. Endothelial nuclear factor kB and the initiation of the atherosclerotic lesion. Lab. Invest. 68, 499-508 (1993).
24. Husebekk, A., Skogen, B., Huskry, G. & Marbaug, G. Transformation of amyloid precursor SAA to protein AA and Incorporation in amyloid fibrils in vivo. Scan. J. Immunol. 21, 283-287 (1985).
25. Kirschner, D., Abraham, C. & Selkoe, D. X-ray diffraction from intraneuronal paired helical filaments and extraneuronal amyloid fibrils in Alzheimer's disease indicates cross-beta conformation. Proc. Natl. Acad. Sci. USA 83, 503-507 (1986).
26. Kimball, M., Sato, A., Richardson, J. Rosen, L. & Low, B. Molecular conformation of erabutoxin b. Biochem. Biophys. Res. Comm. 88, 950-956 (1979).
27. Dickson, D. Microglia in Alzheimer's disease and transgenic models. Am. J. Pathol. 154, 1627-1631 (1999).
28. Uchlhara, T., Adlyama, H., Kando, H. & Ikeda, K. Activated microglial cells are colocalized with perivascular deposits of amyloid beta-peptide in Alzheimer's disease brain. Stroke 28, 1948-1950 (1997).
29. Yan, S-D. Et al. Amyloid beta peptide-RAGE interaction elicits neuronal expression of M-CSF: a proinflammatory pathway in Alzheimer disease. Proc. Natl. Acad. Sci. USA 94, 5296-5301 (1997).
30. Fixe, P. & Praloran, V. M-CSF: haematopoietic growth factor or inflammatory cytokine? Cytokine 10, 32-37 (1998).
31. Hamilton, J. CSF-1 signal transduction, J. Leukoc. Biol. 62:145-155 (1997).
32. Hume, D. et al. Regulation of CSF-1 receptor expression. Mol. Reprod. Dev. 46, 46-52 (1997).
33. Ando, Y. et al. oxidative stress Is found in amyloid deposits in systemic amyloidosis. Biochem. Biophys. Res. Commun. 232, 497-502 (1997).
34. Ritthaler, U. et al. Expression of RAGE in peripheral occlusive vascular disease. Am. J. Pathol. 146, 688-694 (1995).
35. Wautier, J-L. Et al. Receptor-mediated endothelial cell dysfunction in diabetic vasculopathy: soluble RAGE blocks hyperpermeability. J. Clin. Invest. 97, 238-243 (1996).
36. Rudderman, N., Williamson, J. & Brownlee, M. Glucose and diabetic vascular disease. FASEB J. 6, 2905-2914 (1992).
37. Brett, J. et al. Tissue distribution of RAGE: expression in smooth muscle, cardiac myocytes, and neural tissue in addition to the vasculature. Am. J. Pathol. 143, 1699-1712 (1993).
38. Hori, 0. et al. RAGE is a cellular binding site for amphoterin: mediation of neurite outgrowth and co-expression of RAGE and amphoterin in the developing nervous system. J. Biol. Chem. 270, 25752-25761 (1995).
39. Schafer, B. & Helzmann, C. The S100 family of EF-hand calcium-binding proteins: functions and pathology. Trends Biochem. Sci. 21, 134-140 (1996).
40. Wang, H. et al. HMG1 as a late mediator of endotoxin lethality in mice. Science 285, 248-251, 1999.
41. Taguchi, A. et al. Blockade of RAGE/amphoterin axis suppresses tumor growth and metastases. Nature (in the press).
42. Prelli, F., Pras. M. & Frangione, B. Degradation and deposition of amyloid A fibrils are tissue specific. Biochemistry 26, 8251-8256 (1987).
43. Klotz, L & Hunston, D. Mathematical models for ligand-receptor binding. J. Biol. Chem. 258, 11442-11445 (1984).

1-41. (canceled)

42. A method for treating a disease involving β-sheet fibril formation in a subject which comprises administering to the subject an amount of a soluble compound which comprises a V-domain of RAGE effective to inhibit binding of the β-sheet fibril to receptor for advanced glycation endproduct, wherein the β-sheet fibril comprises amylin, amyloid A, transthyretin, cystatin C, or gelsolin, so as to thereby treat the disease involving β-sheet fibril formation in the subject.

43. The method of claim 42, wherein the subject is a mammal.

44. The method of claim 43, wherein the mammal is a human being.

45. The method of claim 42, wherein the administration is intralesional, intraperitoneal, intramuscular, intravenous, liposome mediated delivery, topical, nasal, oral, anal, ocular or otic delivery.

46. The method of claim 42, wherein the β-sheet fibril comprises a peptide capable of forming amyloid.

47. The method of claim 42, wherein the soluble compound comprises the V-domain of RAGE linked to an antibody or a portion of an antibody.

48. The method of claim 42, wherein the soluble compound comprises the V-domain of RAGE linked to a portion of an antibody.

49. The method of claim 42, wherein the portion of the antibody is a F_{ab} fragment.

50. The method of claim 42, wherein the portion of the antibody is an F_{c} fragment.

51. The method of claim 42, wherein the β-sheet fibril comprises amylin.

52. The method of claim 42, wherein the β-sheet fibril comprises amyloid A.

53. The method of claim 42, wherein the β-sheet fibril comprises transthyretin.

54. The method of claim 42, wherein the β-sheet fibril comprises cystatin C.

55. The method of claim 42, wherein the β-sheet fibril comprises gelsolin.
